(19) United States
(12) Patent Application Publication
(10) Pub. No.: US 2009/0203051 A1
(43) Pub. Date: Aug. 13, 2009

(54) TARGETS IN BREAST CANCER FOR PROGNOSIS OR THERAPY

(75) Inventors: Joe W. Gray, San Francisco, CA (US); Jane Fridlyand, Berkeley, CA (US); Richard Neve, San Mateo, CA (US); Paul Spellman, Benicia, CA (US); Koei Chin, Foster City, CA (US); Zhi Hu, El Cerrito, CA (US); Frederic Waldman, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/330,386

(22) Filed: Dec. 8, 2008

Related U.S. Application Data
(63) Continuation-in-part of application No. PCT/US2007/070908, filed on Jun. 11, 2007.
(60) Provisional application No. 60/812,704, filed on Jun. 9, 2006.

(51) Int. Cl. A61K 31/7088; C12Q 1/68; C40B 30/04; G01N 33/574; G01N 33/68; C12N 5/06

(52) U.S. Cl. 435/7.23; 435/6; 506/9; 436/86; 435/375

(57) ABSTRACT

Cancer markers are developed to detect diseases characterized by increased expression of apoptosis-suppressing genes, such as aggressive cancers. Genome wide analyses of genome copy number and gene expression in breast cancer revealed 66 genes in the human chromosomal regions, 8p11, 11q13, 17q12, and 20q13 that were amplified. Diagnosis and assessment of amplification levels of genes shown to be amplified are useful in prediction of patient outcome of a of patient's response and drug resistance in breast cancer. Certain genes were found to be high priority therapeutic targets by the identification of recurrent aberrations involving genome sequence, copy number and/or gene expression are associated with reduced survival duration in certain diseases and cancers, specifically breast cancer. Inhibitors of these genes will be useful therapies for treatment of these non-responsive cancers.

### CROSS-REFERENCE TO RELATED APPLICATIONS

This application is a continuation-in-part of PCT application no. PCT/US2007/070908, filed Jun. 11, 2007, which claims priority to U.S. provisional patent application No. 60/812,704, filed on Jun. 9, 2006, each of which applications is hereby incorporated by reference in its entirety.

### STATEMENT OF GOVERNMENTAL SUPPORT

This invention was made during work supported by the National Cancer Institute, through Grants CA 58207 and CA 112970, and during work supported by the U.S. Department of Energy under Contract No. DE-AC03-76SF00098, now DE-AC02-05CH11231. The government has certain rights in this invention.

### REFERENCE TO ATTACHED TABLES AND SEQUENCE LISTINGS

This application incorporates by reference in their entirety the attached tables and sequence listing. Tables 1-6 and 8-10 are appended after the claims and are incorporated by reference. The content of the accompanying sequence listing is incorporated herein by reference in its entirety.

### FIELD OF THE INVENTION

The present invention relates to markers and chromosomal amplification correlated to disease, particularly malignant disease such as breast cancer. More specifically, the present invention relates to using cancer markers and chromosomal region analyses for the prediction of patient outcome in breast cancer patients. The present invention also relates to markers and therapeutics targeting in vivo drug resistance. More specifically, the present invention relates to the diagnosis and treatment using cancer markers and therapeutics which target drug resistance in breast cancer patients with low survival rates.

### BACKGROUND OF THE INVENTION

Breast cancer is one of the most common malignancies among women and shares, together with lung carcinoma, the highest fatality rate of all cancers affecting females. The current treatment of the breast cancer is limited to a very invasive, total or partial mastectomy, radiation therapy, or chemotherapy, later two resulting in serious undesirable side effects.

It is now well established that breast cancers progress through accumulation of genomic (Albertson et al., 2003; Knuutila et al., 2000) and epigenomic (Baylin and Herman, 2000; Jones, 2005) aberrations that enable development of aspects of cancer pathophysiology such as reduced apoptosis, unchecked proliferation, increased motility, and increased angiogenesis (Hanahan and Weinberg, 2000). Discovery of the genes that contribute to these pathophysiologies when deregulated by recurrent aberrations is important to understanding mechanisms of cancer formation and progression and to guide improvements in cancer diagnosis and treatment.

Analyses of expression profiles have been particularly powerful in identifying distinctive breast cancer subsets that differ in biological characteristics and clinical outcome (Perou et al., 1999; Perou et al., 2000; Sorlie et al., 2001; Sorlie et al., 2003). For example, unsupervised hierarchical clustering of microarray derived expression data have identified intrinsically variable gene sets that distinguish five breast cancer subtypes—basal-like, luminal A, luminal B, ERBB2 and normal breast-like. The basal-like and ERBB2 subtypes have been associated with strongly reduced survival durations in patients treated with surgery plus radiation (Perou et al., 2000; Sorlie et al., 2001) and some studies have suggested that reduced survival duration in poorly performing subtypes is caused by an inherently high propensity to metastasize (Ramaswamy et al., 2003). These analyses already have led to the development of multi-gene assays that stratify patients into groups that can be offered treatment strategies based on risk of progression (Esteva et al., 2005; Gianni et al., 2005; van 't Veer et al., 2002). However, the predictive power of these assays is still not as high as desired and the assays have not been fully tested in patient populations treated with aggressive adjuvant chemotherapies.

Analyses of breast tumors using fluorescence in situ hybridization (Al-Kuraya et al., 2004; Kallioniemi et al., 1992; Press et al., 2005; Tanner et al., 1994) and comparative genomic hybridization (Kallioniemi et al., 1994; Loo et al., 2004; Naylor et al., 2005; Pollack et al., 1999) show that breast tumors also display a number of recurrent genome copy number aberrations including regions of high level amplification that have been associated with adverse outcome (Al-Kuraya et al., 2004; Cheng et al., 2004; Isola et al., 1995; Jain et al., 2001; Press et al., 2005). This raises the possibility of improved patient stratification through combined analysis of gene expression and genome copy number (Barlund et al., 2000; Pollack et al., 2002; Ray et al., 2004; Yi et al., 2005). In addition, several studies of specific chromosomal regions of recurrent abnormality at 17q12 (Kauraniemi et al., 2001; Kauraniemi et al., 2003) and 8p11 (Gelsi-Boyer et al., 2005; Ray et al., 2004) show the value of combined analysis of genome copy number and gene expression for identification of genes that contribute to breast cancer pathophysiology by deregulating gene expression.

Nevertheless, there is a continued need for further understanding of the genes, and of the chromosomal aberration(s) that occur in cancer, for example breast cancer.

### BRIEF SUMMARY OF THE INVENTION

Disclosed herein are roles of genome copy number abnormalities (CNAs) in breast cancer pathophysiology by identifying associations between recurrent CNAs, gene expression and clinical outcome in a set of aggressively treated early stage breast tumors. It shows that the recurrent CNAs differ between tumor subtypes defined by expression pattern and that stratification of patients according to outcome can be improved by measuring both expression and copy number; especially high level amplification. Sixty-six genes (set forth in Table 3) deregulated by the high level amplifications are therapeutic targets; nine of these genes (FGFR1, IKBKB, ERBB2, PROSC, ADAM9, FNTA, ACA CA, PNMT, and NR1D1) are “druggable.” Low level CNAs appear to contribute to cancer progression by altering RNA and cellular metabolism.

As used herein gene amplification is used in a broad sense. It comprises an increase of gene copy number; it can also comprise assessment amplification of the gene product. Thus levels of gene expression, as well as corresponding protein expression can be evaluated. In the embodiments that follow, it is understood that assessment of gene expression can be used to assess level of gene product such as RNA or protein.

Thus, embodiments of the invention include: A method for prognosing the outcome of a patient with breast cancer, said method comprising: providing breast cancer tissue from the patient; determining from the provided tissue, the level of gene amplification or gene expression for at least one gene set forth in Table 3; identifying that the at least one gene or gene product is amplified; whereby, when the at least one gene or gene product is amplified, this is an indication that the patient has the predicted disease free survival or probability for distant recurrence set forth in Table3. This method can comprise that the gene or gene product is ACACA (SEQ ID NOs: 1, 2), ADAM9 (SEQ ID NOs: 3-8), ERBB2 (SEQ ID NOs: 9-14), FGFR1 (SEQ ID NOs: 15, 16), FNTA (SEQ ID NOs: 17, 18), IKBKB (SEQ ID NOs: 19, 20), NR1D1 (SEQ ID NOs: 21, 22), PNMT (SEQ ID NOs: 23, 24), or PROSC (SEQ ID NOs: 25, 26); in particular PROSC (SEQ ID NOs: 25, 26), ADAM9 (SEQ ID NOs: 3-8), FNTA (SEQ ID NOs: 17, 18), ACACA (SEQ ID NOs: 1, 2), PNMT (SEQ ID NOs: 23, 24), or NR1D1 (SEQ ID NOs: 21, 22). In one preferred embodiment, the gene, ADAM9 (SEQ ID NOs: 3, 5 and 7) is a therapeutic target. In certain embodiments, there is a proviso that the gene or gene product is not ERBB2 (SEQ ID NOs: 9-14), FGFR1 (SEQ ID NOs: 15, 16), or IKBKB (SEQ ID NOs: 19, 20). The detecting step can comprise use a of methodology selected from the group consisting of quantitative PCR, FISH, array CGH, quantitative PCR, in situ hybridization for RNA, immunohistochemistry and reverse phase protein lysate arrays for protein. In some embodiments, the gene or gene product is FGF3 (SEQ ID NOs: 65, 66), PPFIA1 (SEQ ID NOs: 69, 70), NEU3 (SEQ ID NOs: 79, 80), CSTF1 (SEQ ID NOs: 117, 118), PCK1 (SEQ ID NOs: 123, 124), VAPB (SEQ ID NOs: 129, 130), GNAS (SEQ ID NOs: 135, 136), BCAS1 (SEQ ID NOs: 115, 116), TMEPA1 (SEQ ID NOs: 125, 126), or STX16 (SEQ ID NOs: 131, 132). In certain embodiments, the gene or gene product is FGF3 (SEQ ID NOs: 65, 66), PPFIA1 (SEQ ID NOs: 69, 70), NEU3 (SEQ ID NOs: 79, 80), GNAS (SEQ ID NOs: 135, 136), TMEPA1 (SEQ ID NOs: 125, 126), STX16 (SEQ ID NOs: 131, 132), or VAPB (SEQ ID NOs: 129, 130). In some embodiments, the breast cancer is a luminal A breast cancer and the gene or gene product is a gene or encoded by a gene at 11q13-14 and/or 20q13, e.g., FGF3 (SEQ ID NOs: 65, 66), PPFIA1 (SEQ ID NOs: 69, 70), NEU3 (SEQ ID NOs: 79, 80), GNAS (SEQ ID NOs: 135, 136), TMEPA1 (SEQ ID NOs: 125, 126), STX16 (SEQ ID NOs: 131, 132), or VAPB (SEQ ID NOs: 129, 130).

An embodiment in accordance with the invention comprises: A method for selecting a patient for treatment with a drug that modulates the expression of a gene set forth in Table 3, said method comprising: providing tissue biopsy from the patient; determining from the provided tissue, the level of gene amplification or gene product expression for a gene set forth in Table 3; identifying that one or more of the genes or gene products is amplified; whereby, when the one or more genes or gene products are amplified, this gene and/or gene product is a candidate for treatment with a drug that modulates the expression of the one or more gene of Table 3 or a drug that affects a protein of Table 3. In certain embodiments, the gene or product is ACACA (SEQ ID NOs: 1, 2), ADAM9 (SEQ ID NOs: 3-8), ERBB2 (SEQ ID NOs: 9-14), FGFR1 (SEQ ID NOs: 15, 16), FNTA (SEQ ID NOs: 17, 18), IKBKB (SEQ ID NOs: 19, 20), NR1D1 (SEQ ID NOs: 21, 22), PNMT (SEQ ID NOs: 23, 24), or PROSC (SEQ ID NOs: 25, 26); in particular PROSC (SEQ ID NOs: 25, 26), ADAM9 (SEQ ID NOs: 3-8), FNTA (SEQ ID NOs: 17, 18), ACACA (SEQ ID NOs: 1, 2), PNMT (SEQ ID NOs: 23, 24), or NR1D1 (SEQ ID NOs: 21, 22).; and in one embodiment, particularly, ADAM9. In certain embodiments, there is a proviso that the gene or gene product is not ERBB2 (SEQ ID NOs: 9-14), FGFR1 (SEQ ID NOs: 15, 16), or IKBKB (SEQ ID NOs: 19, 20). In some embodiments, the gene or gene product is FGF3 (SEQ ID NOs: 65, 66), PPFIA1 (SEQ ID NOs: 69, 70), NEU3 (SEQ ID NOs: 79, 80), CSTF1 (SEQ ID NOs: 117, 118), PCK1 (SEQ ID NOs: 123, 124), VAPB (SEQ ID NOs: 129, 130), GNAS (SEQ ID NOs: 135, 136), BCAS1 (SEQ ID NOs: 115, 116), TMEPA1 (SEQ ID NOs: 125, 126), or STX16 (SEQ ID NOs: 131, 132). In certain embodiments, the gene or gene product is FGF3 (SEQ ID NOs: 65, 66), PPFIA1 (SEQ ID NOs: 69, 70), NEU3 (SEQ ID NOs: 79, 80), GNAS (SEQ ID NOs: 135, 136), TMEPA1 (SEQ ID NOs: 125, 126), STX16 (SEQ ID NOs: 131, 132), or VAPB (SEQ ID NOs: 129, 130). In some embodiments, the breast cancer is a luminal A breast cancer and the gene or gene product is a gene or encoded by a gene at 11q13-14 and/or 20q13, e.g., FGF3 (SEQ ID NOs: 65, 66), PPFIA1 (SEQ ID NOs: 69, 70), NEU3 (SEQ ID NOs: 79, 80), GNAS (SEQ ID NOs: 135, 136), TMEPA1 (SEQ ID NOs: 125, 126), STX16 (SEQ ID NOs: 131, 132), or VAPB (SEQ ID NOs: 129, 130). The determining step can comprise use a of methodology selected from the group consisting of quantitative PCR, FISH, array CGH, quantitative PCR, in situ hybridization for RNA, immunohistochemistry and reverse phase protein lysate arrays for protein.

An embodiment of the invention comprises: A method for treatment of a patient with breast cancer, said method comprising: providing tissue biopsy from the patient; determining from the provided tissue, the level of gene amplification or level of gene product for a gene set forth in Table 3; identifying that one or more of the genes or gene products is amplified; whereby, when the one or more genes or gene products are amplified, this patent is treated with a drug that modulates the expression of the one or more gene or a drug that affects the gene product. In certain embodiments, the gene or gene product is ACACA (SEQ ID NOs: 1, 2), ADAM9 (SEQ ID NOs: 3-8), ERBB2 (SEQ ID NOs: 9-14), FGFR1 (SEQ ID NOs: 15, 16), FNTA (SEQ ID NOs: 17, 18), IKBKB (SEQ ID NOs: 19, 20), NR1D1 (SEQ ID NOs: 21, 22), PNMT (SEQ ID NOs: 23, 24), or PROSC (SEQ ID NOs: 25, 26); in particular PROSC (SEQ ID NOs: 25, 26), ADAM9 (SEQ ID NOs: 3-8), FNTA (SEQ ID NOs: 17, 18), ACACA (SEQ ID NOs: 1, 2), PNMT (SEQ ID NOs: 23, 24), or NR1D1 (SEQ ID NOs: 21, 22).; or more particularly in one embodiment, ADAM9. In certain embodiments there is a proviso that the gene or gene product is not ERBB2(SEQ ID NOs: 9-14), FGFR1 (SEQ ID NOs: 15, 16), or IKBKB (SEQ ID NOs: 19, 20). In some embodiments, the gene or gene product is FGF3 (SEQ ID NOs: 65, 66), PPFIA1 (SEQ ID NOs: 69, 70), NEU3 (SEQ ID NOs: 79, 80), CSTF1 (SEQ ID NOs: 117, 118), PCK1 (SEQ ID NOs: 123, 124), VAPB (SEQ ID NOs: 129, 130), GNAS (SEQ ID NOs: 135, 136), BCAS1 (SEQ ID NOs: 115, 116), TMEPA1 (SEQ ID NOs: 125, 126), or STX16 (SEQ ID NOs: 131, 132). In certain embodiments, the gene or gene product is FGF3 (SEQ ID NOs: 65, 66), PPFIA1 (SEQ ID NOs: 69, 70), NEU3 (SEQ ID NOs: 79, 80), GNAS (SEQ ID NOs: 135, 136), TMEPA1 (SEQ ID NOs: 125, 126), STX16 (SEQ ID NOs: 131, 132), or VAPB (SEQ ID NOs: 129, 130). In some embodiments, the breast cancer is a luminal A breast cancer and the gene or gene product is a gene or encoded by a gene at 11q13-14 and/or 20q13, e.g., FGF3 (SEQ ID NOs: 65, 66), PPFIA1 (SEQ ID NOs: 69, 70), NEU3 (SEQ ID NOs: 79, 80), GNAS (SEQ ID NOs: 135, 136), TMEPA1 (SEQ ID NOs: 125, 126), STX16 (SEQ ID NOs: 131, 132), or VAPB (SEQ ID NOs: 129, 130). In one embodiment the drug is an antisense sequence for a gene of Table 3, and the particular antisense sequence corresponds to the one or more amplified genes identified in the identifying step. The determining step can comprise use a of methodology selected from the group consisting of quantitative PCR, FISH, array CGH, quantitative PCR, in situ hybridization for RNA, immunohistochemistry and reverse phase protein lysate arrays for protein.

Another embodiment of the invention comprises: A method for identifying a moiety that modulates a protein, said method comprising: providing a protein selected from the group consisting of PROSC (SEQ ID NO: 26), ADAM9 (SEQ ID NOs: 4, 6, or 8), FNTA (SEQ ID NO: 18), ACACA (SEQ ID NO: 2), PNMT (SEQ ID NO: 24), or NR1D1 (SEQ ID NO: 22); screening the provided protein with a candidate moiety; determining whether the candidate moiety modules (e.g., alters function or expression) of the protein; and, selecting a moiety that modules the protein. A further embodiment comprises: A method for modulating a PROSC (SEQ ID NO: 26), ADAM9 (SEQ ID NOs: 4, 6 or 8), FNTA (SEQ ID NO: 18), ACACA (SEQ ID NO: 2), PNMT (SEQ ID NO: 24), or NR1D1 (SEQ ID NO: 22) protein in a living cell, said method comprising: providing a moiety that modulates the protein; administering the moiety to a living cell that expresses PROSC, ADAM9, FNTA, ACACA, PNMT, or NR1D1 protein corresponding to the moiety; whereby, PROSC, ADAM9, FNTA, ACACA, PNMT, or NR1D1 protein in the cell is modulated.

Another embodiment of the invention comprises a method for prognosing the outcome of a patient with breast cancer, said method comprising: providing breast cancer tissue from the patient; determining from the provided tissue, the level of gene deletion for at least one gene from amplicon 8p11-12; identifying that the at least one gene is deleted; whereby, when the at least one gene is deleted, this is an indication that the patient has the predicted disease free survival or probability for distant recurrence set forth in Table3. In certain embodiments, the at least one gene from amplicon 8p11-12 is selected from the chromosome 8 genes set forth in Table 3. The determining step can comprise use a of methodology selected from the group consisting of quantitative PCR, FISH, array CGH, quantitative PCR, in situ hybridization for RNA, immunohistochemistry and reverse phase protein lysate arrays for protein.

### BRIEF DESCRIPTION OF THE DRAWINGS

FIG. 1. Recurrent abnormalities in 145 primary breast tumors.

FIG. 1(a). Frequencies of genome copy number gain and loss plotted as a function of genome location with chromosomes 1pter to the left and chromosomes 22qter and X to the right. Vertical lines indicate chromosome boundaries and vertical dashed lines indicate centromere locations. Positive and negative values indicate frequencies of tumors showing copy number increases and decrease respectively with gain and loss as described in the methods.

FIG. 1(b). Frequencies of tumors showing high level amplification. Data are displayed as described in FIG. 1(a).

FIG. 1(c-j). Frequencies of tumors showing significant copy number gains and losses as defined in FIG. 1(a) (upper member of each pair) or high level amplifications as defined in FIG. 1(b) (lower member of each pair) in tumor subtypes defined according to expression phenotype; FIG. 1(c) & FIG. 1(d), basal-like; FIG. 1(e) & FIG. 1(f), ERBB2;

FIG. 1(g) & FIG. 1(h), luminal A; FIG. 1(i) & FIG. 1(j), luminal B. Data are displayed as described in FIG. 1(a).

FIG. 2. Unsupervised hierarchical clustering of genome copy number profiles measured for 145 primary breast tumors. Green indicates increased genome copy number and red indicates decreased genome copy number. The three major genomic clusters from left to right are designated 1q/16q, Complex and Amplifying. The bar to the left indicates chromosome locations with chromosome 1pter to the top and 22qter and X to the bottom. The locations of the odd numbered chromosomes are indicated. The upper color bars indicate biological and clinical aspects of the tumors. Color codes are indicated at the bottom of the figure. Dark blue indicates positive status, light blue indicates negative status for Nodes, ER, PR and p53 expression. For Ki67, dark blue=fraction >0.1 and light blue=fraction <0.1. For size, light blue indicates size <2.2 cm, and dark blue indicates size >2.2 cm. Color codes for the expression bar are orange=luminal A, dark blue=normal breast-like, light blue=ERBB2, green=basal-like, yellow=luminal B.

FIG. 3. Kaplan Meyer plots showing survival in breast tumor subclasses.

FIG. 3(a). Disease specific survival in 130 breast cancer patients whose tumors were defined using expression profiling to be basal-like (third curve down), luminal A (top curve), luminal B (second curve from top) and ERBB2 class (bottom curve).

FIG. 3(b). Disease-specific survival of patients with tumors classified by genome copy number aberration analysis as 1q/16q (top curve), Complex (red) and Amplifying (blue).

FIG. 3(c). Survival of patients with (bottom curve) and without (top curve) amplification at any region of recurrent amplification.

FIG. 3(d). Survival of patients whose tumors were defined using expression profiling to be luminal A tumors with (bottom curve) and without (top curve) amplification at 8p11-12, 11q13, and/or 20q.

FIG. 3(e). Survival of patients whose tumors that were not amplified at 8p11-12 and that had normal (top curve) or reduced (bottom curve) genome copy number at 8p11-12.

FIG. 3(f). Survival of patients whose tumors had normal (top curve) or abnormal (bottom curve) genome copy number at 8p11-12.

FIG. 4. Results of unsupervised hierarchical clustering of 130 breast tumors using intrinsically variable gene expression but excluding any transcripts whose levels were significantly associated with genome copy number. Red indicates increased expression and green indicates reduced expression.

FIG. 5. Comparison of recurrent genome aberrations in 145 primary breast tumors with low-level genome copy number aberrations selected in human mammary epithelial cells during passage through telomere crisis (Chin et al., 2004).

FIG. 5(a). Frequencies of genome copy number gain and loss plotted as described in FIG. 1(a).

FIG. 5(b). Array CGH analyses of genome copy number for human mammary epithelial cells at passages 16 and 21 before transition through telomere crisis (upper two traces) and at passages 28 and 44 after immortalization (lower two traces) (Chin et al., 2004).

FIG. 6. Unsupervised hierarchical clustering of expression profiles measured for 148 tumors based on a published set of intrinsically variably genes, URL:<http://www.pnas.org/cgi/content/full 100/14/8418>, matched by UniGene ID 280 unique out of 464 gene probes in Affymetrix GeneChip). Tumor IDs under dendogram were color-coded red; basaloid, pink; ERBB2, blue; luminal A and light blue; luminal B) based on the closest distance to each subtype in 79 tumors of Stanford samples. Expression values in the cluster diagram were median centered for each gene. Similarly color-coded maker gene names for each subtype were displayed with UniGene IDs on the right of cluster diagram. These marker genes were highly expressed in the each subtype indicated in red in the cluster diagram. For redundant genes with correlation >0.45, expression values were averaged. ER positive and negative status is indicated in yellow and blue respectively under tumor ID, which corresponds well to erbB2 and basal type tumors.

FIG. 7. Graphs showing that transient transfection of siRNA for ADAM9 into (a) T47D, (b) BT549, (c) SUM52PE breast cancer cell lines strongly inhibits growth in breast cancer cells.

FIG. 8. Graphs showing that silencing of ADAM9 decreased proliferation of breast cancer cells (a) BT549 and (b) SUM52PE, but not (c) normal cells MCF10A.

FIG. 9. Down-regulation of ADAM9 by siRNA increased apoptosis in breast cancer cells as determined by detecting Yo-Pro staining.

FIG. 10. By detecting cell survival rates, growth inhibition was achieved by >30 nM siRNA in BT549 and SUM52PE breast cancer cells.

FIG. 11. siFGF3, siPPFIA1 and siNEU3 specifically inhibited cell growth in highly amplified cell lines. Cell viability was measured by the Luminescence cell viability assay (Promega Inc.) following treatment with siRNAs for 72 hours. The inhibition rate was achieved by comparison to non-target negative controls (siControl).

FIG. 12. siFGF3, siPPFIA1 and siNEU3 induced cell apoptosis in 11q13 highly amplified cell lines, but not in not-amplified cell line. Cell apoptosis was assayed using YoPro-1 and Hoechst staining with the Cellomics high content scanning instrument in 72 hours post transfection. The fold of apoptosis was achieved by normalizing to control siRNA(siControl).

FIG. 13. shRNAs can efficiently knock down FGF3, NEU3 and PPFIA1 in breast cancer cells. Protein levels were confirmed by western blot after infection of breast cancer cells with shRNAs (five shRNAs targeting different sequences/gene). Actin was the loading control. Each gene had at least one shRNA that could efficiently knock down the target gene.

FIG. 14. Knockdown of FGF3, PPFIA1 and NEU3 by shRNAs induces cell apoptosis CAMA1 cells with Caspase3 Glo assay(Promega) and YoPro/Hochest double staining.

FIG. 15. Silencing of FGF3, PPFIA1 and NEU3 by shRNAs inhibit cell growth in 3D culture. Cells that had knocked down FGF3, PPFIA1 and NEU3 proteins (with shRNAs_(#38160 shFGF3, #2969 shPPFIA1 and #5149 shNEU3 respectively) were evaluated in 3D culture. The HCC1954 and CAMA1 cells with shFGF3, shPPFIA1 and/or shNEU3 were very unhealthy and died off. The colonies were much smaller for cells with shFGF3 shPPFIA1 and/or shNEU3 than control cells. The morphology also changed compared to control cells, which had typical mass-like morphology of HCC1954 (grape like morphology of CAMA 1 cells).

FIG. 16. Synergistic effects on combinational knockdown of NEU3 and PPFIA1 genes at 11q13 amplicon. Cell viability/proliferation was evaluated by Cell Titer-Glo luminescent cell viability assay (Promega) after cells had been infected with shRNA lentivirus for 6 days. The cell viability percentage was normalized to control shRNA. Cell apoptosis was analyzed with YoPro-1 and Hoechst staining using the Cellomics high content scanning instrument after cells had been infected with shRNA lentivirus for 6 days.

FIG. 17. Candidate Therapeutics on 20q13 amplicon. The cell viability was measured by Luminescence cell viability assay (Promega Inc.) following treatment with siRNAs for 72 hours. The inhibition rate was determined in comparison to non-target negative control(siControl).

FIG. 18. GNAS, STX16, TMEPA1 and VAPB siRNAs inhibit cell proliferation by BrdU and Hoeschst staining.

FIG. 19. GNAS, STX16, TMEPA1 and VAPB siRNAs inhibit apoptosis by YoPro-1 and Hoeschst staining.

FIG. 20. Caspase3 activity increased in SUM52PE cells treated with GNAS, STX16, TMEPA1, and VAPB siRNAs.

FIG. 21. GNAS siRNAs knocked down Gs transcripts in breast cancer cell lines.

### BRIEF DESCRIPTION OF THE TABLES

Table 1. Univariate and multivariate associations for individual amplicons and/or disease specific survival and distant recurrence. Also shown are the chromosomal positions of the beginning and ends of the amplicons and the flanking clones. Associations are shown for the entire sample set and for luminal A tumors (univariate associations only).

Table 2. Associations of genomic variables with clinical features.

Table 3. Functional characteristics of 66 genes; these genes are in recurrent amplicons associated with reduced survival duration in breast cancer. Functional annotation was based on the Human Protein Reference Database (http://hprd.org). Genes highlighted in dark gray are associated with reduced survival duration or distant recurrence when over expressed in non-Amplifying tumors. Genes highlighted in light gray are significantly associated with reduced survival duration or distant recurrence (p<0.05) when down regulated in non-Amplifying tumors. Distances to sites of recurrent viral integration were determined from published information (Akagi et al., 2004). The last column identifies genes having predicted protein folding characteristics indicating that they are druggable (see, e.g., Russ and Lampel, 2005).

Table 4. Univariate p-values with the corresponding 95% confidence intervals for associations with disease-specific survival and distant recurrence endpoints and the corresponding multivariate results for those found to be significant in univariate analyses (p<0.05) for at least one of the clinical end points. Only variables individually significant at p<0.05 for at least one of the two end points are included in the multivariate regression. Stage and SBR Grade are treated as continuous variables rather than factors. In each column pair, the left subcolumn lists results for disease-specific survival and the right subcolumn lists results for time to distant recurrence.

Table 5. Comparison of the association between expression subtypes and survival duration in 3 datasets. Log-likelihood ratio test p-value is shown for each model. Basal is the reference in all models. Multivariate models include size and nodal status. In multivariate analyses, the first value shown in each cell is the p-value and the second is the ratio of the medians in the compared groups.

Table 6. Identities of 1432 gene transcripts showing significant associations between genome copy numbers measured using array CGH and transcript levels measured using Affymetrix U133A expression arrays in 101 primary breast tumors. Data will be available through CaBIG and a public web site.

Table 7. The set of genes in Table 3, shown with the corresponding GenBank Accession numbers and the SEQ ID NOs assigned for the gene and gene products.

Table 8. Sequences of siRNAs targeting various human genes encoded by amplicon 11q13.

Table 9. Sequences of shRNAs targeting human NEU3, FGF3 and PPFIA1 genes.

Table 10. Sequences of siRNAs targeting various human genes encoded by amplicon 20q13.

### DETAILED DESCRIPTION OF THE PREFERRED EMBODIMENTS

In order to further the understanding of the genes, and of the chromosomal aberration(s) that occur in cancer, for example breast cancer we performed combined analyses of genome copy number and gene expression to identify genes that contribute to breast cancer pathophysiology with emphasis on those that are associated with poor response to current therapies.

By associating clinical endpoints with genome copy number and gene expression, we showed strong associations between expression subtype and genome aberration composition and we identified four human chromosomal regions (8p11-12, 11q13-14, 17q12 and/or 20q13) of recurrent amplification associated with poor outcome in treated patients. Gene expression profiling revealed 66 genes (see, e.g., Table 3 and Table 7) in these regions of amplification whose expression levels were deregulated by the high-level amplifications. We also found a surprising association between low level CNAs (genome copy number abnormality CNA) and up-regulation of genes associated with RNA and protein metabolism that may suggest a new mechanism by which these aberrations contribute to cancer progression.

We disclose a comprehensive analysis of gene expression and genome copy number in aggressively treated primary human breast cancers performed in order to identify (a) genomic events that are assayed to better stratify patients according to clinical behavior, (b) identify how molecular aberrations contribute to breast cancer pathogenesis and (c) discover genes that are therapeutic targets in patients that do not respond well to current therapies.

Molecular Markers that Predict Outcome

We focused in this study on combined analyses of genome copy number and gene expression in tumors from patients who had aggressive treatment with surgery, radiation of the surgical margins, and hormonal therapy for ER positive disease and aggressive adjuvant chemotherapy as indicated (typically adriamycin and cytoxan but not including Trastuzumab). Analyses of markers in the context of this treatment regimen allowed us to identify those that predicted outcome in patients whose tumors were treated more aggressively than in previously published studies (Esteva et al., 2005; Gianni et al., 2005; van 't Veer et al., 2002). Our analyses of this aggressively treated patient cohort revealed two important associations.

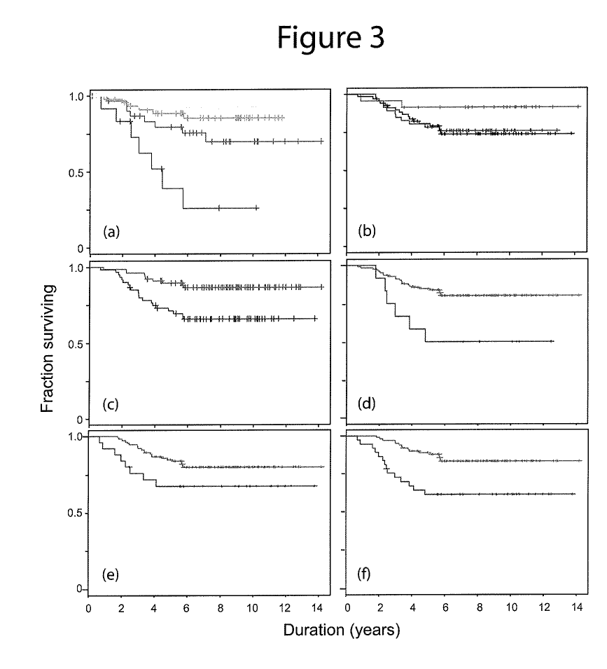

First, we found that the survival of patients with tumors classified as basal-like according to expression pattern did not have significantly worse outcome than patients with luminal or normal-like tumors in this tumor set, unlike previous reports (van 't Veer et al., 2002; van de Vijver et al., 2002) (see FIG. 3(a)). However, patients with ERBB2 positive tumors did do worse (significantly increased death from disease and shorter recurrence-free survival; p<0.001 and p<0.01 respectively, log-rank test) in accordance with the earlier studies. This suggests that the aggressive chemotherapy employed for treatment of the predominantly ER negative basal-like tumors increased survival duration in these patients relative to patients with tumors in the other subgroups. Thus, outcome for patients with basal-like tumors may not be as bad as indicated by earlier prognostic studies of patient populations that did not receive aggressive chemotherapy for progressive disease. This result emphasizes the need to interpret the performance of molecular markers for patient stratification in the context of specific treatment regimens.

Secondly, we found that aggressively treated patients with high level amplification had worse outcome than did patients without amplification (see FIG. 3(c)). This is consistent with earlier CGH and single locus analyses of associations of amplification with poor prognosis (Al-Kuraya et al., 2004; Blegen et al., 2003; Callagy et al., 2005; Gelsi-Boyer et al., 2005; Weber-Mangal et al., 2003). Moreover, the presence of high level amplification was an indicator of poor outcome, even within patient subsets defined by expression profiling. This was particularly apparent for luminal A tumors as illustrated in FIG. 3(d) where patients whose tumors had high level amplification at 8p11-12, 11q13-14 or 20q13 did significantly worse than patients without amplification. This shows that that stratification according to both expression level and copy number will identify patients that respond poorly to current therapeutic treatment strategies.

### Mechanisms of Disease Progression

Our combined analyses of genome copy number and gene expression showed substantial differences in recurrent genome abnormality composition between tumors classified according to expression pattern and revealed that over 10% of the genes interrogated in this study had expression levels that were highly significantly associated with genome copy number changes. Most of the gene expression changes were associated with low level changes in genome copy number, but 66 were deregulated by the high level amplifications associated with poor outcome (see Table 3), as defined as having a multiple testing corrected p-value of less than 0.05. These analyses provide evidence of: the etiology of breast cancer subtypes, mechanisms by which the low-level copy number changes contribute to cancer pathogenesis and identify a suite of genes that contribute to cancer pathophysiology when over expressed as a result of high level amplification.

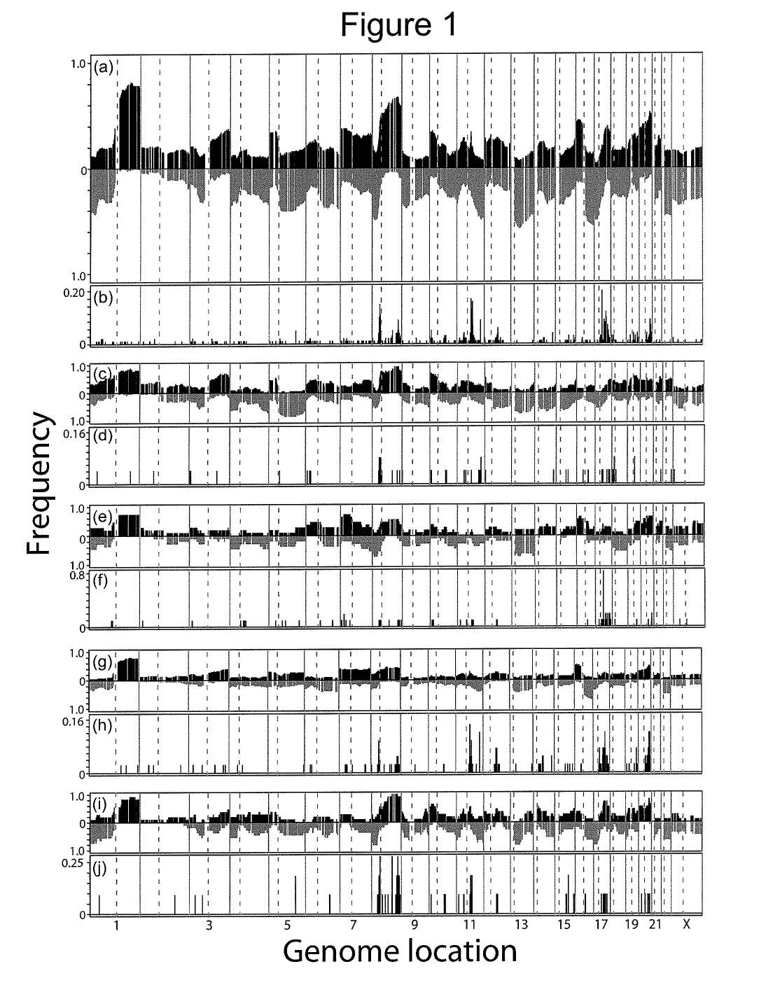

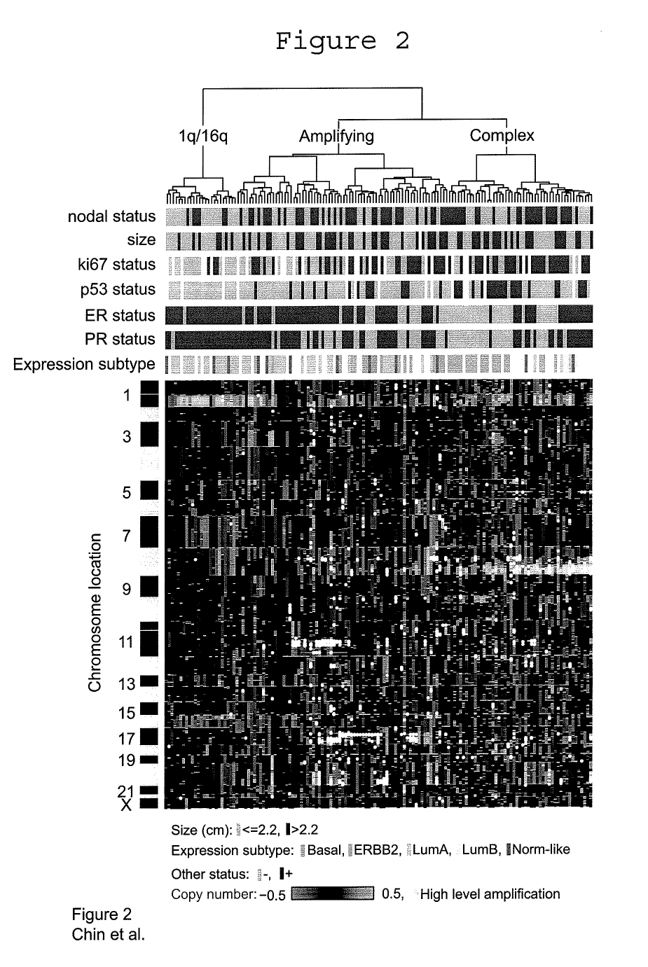

Breast cancer subtypes. FIGS. 1 and 2 show that recurrent genome copy number aberrations differ substantially between tumors classified according to expression pattern as described previously (Perou et al., 1999). Basal-like tumors carried genome aberrations similar to those reported for tumors arising in BRCA1 carriers. High level amplifications were rare in these tumors. ERBB2 tumors were amplified at and distal to the ERBB2 locus on chromosome 17 but amplifications of regions on other chromosomes were rare in this tumor subset(Isola et al., 1999). Luminal A tumors carried frequent gains at 1q and 16p and losses at 16q and carried recurrent amplifications involving 8p11-12, 11q13-14, 17q11-12 and 20q13. Luminal B tumors showed many regions of genome copy number abnormality (CNA) as well as frequent amplification of three regions of chromosomes 8.

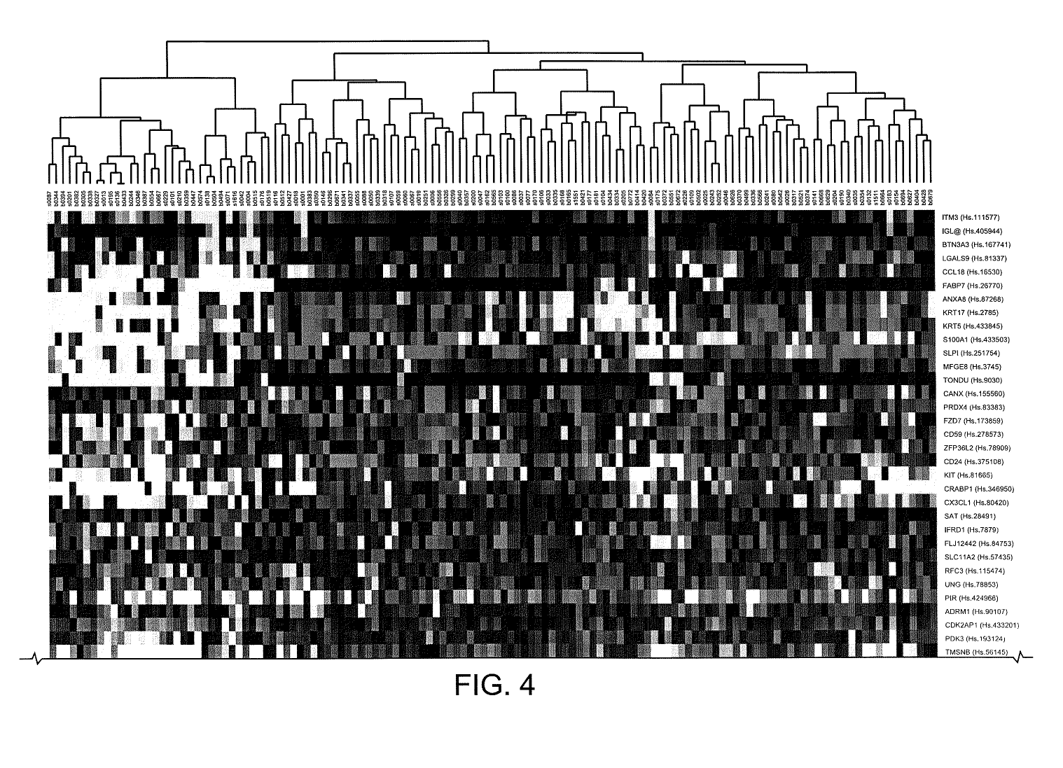

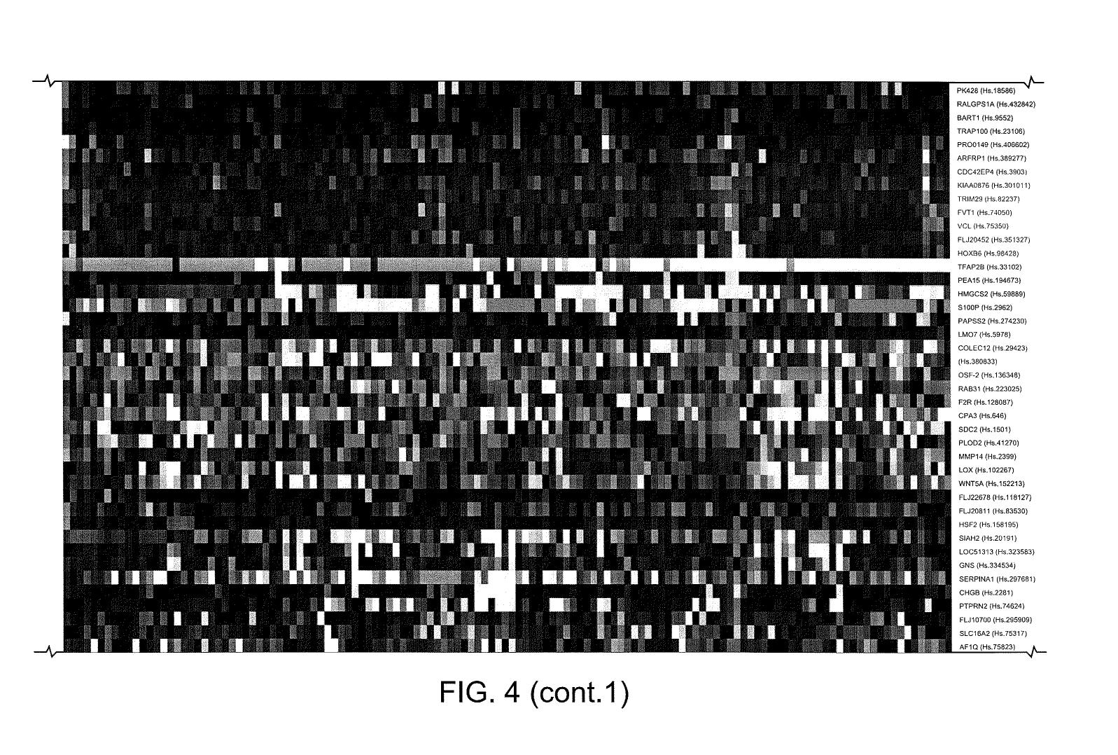

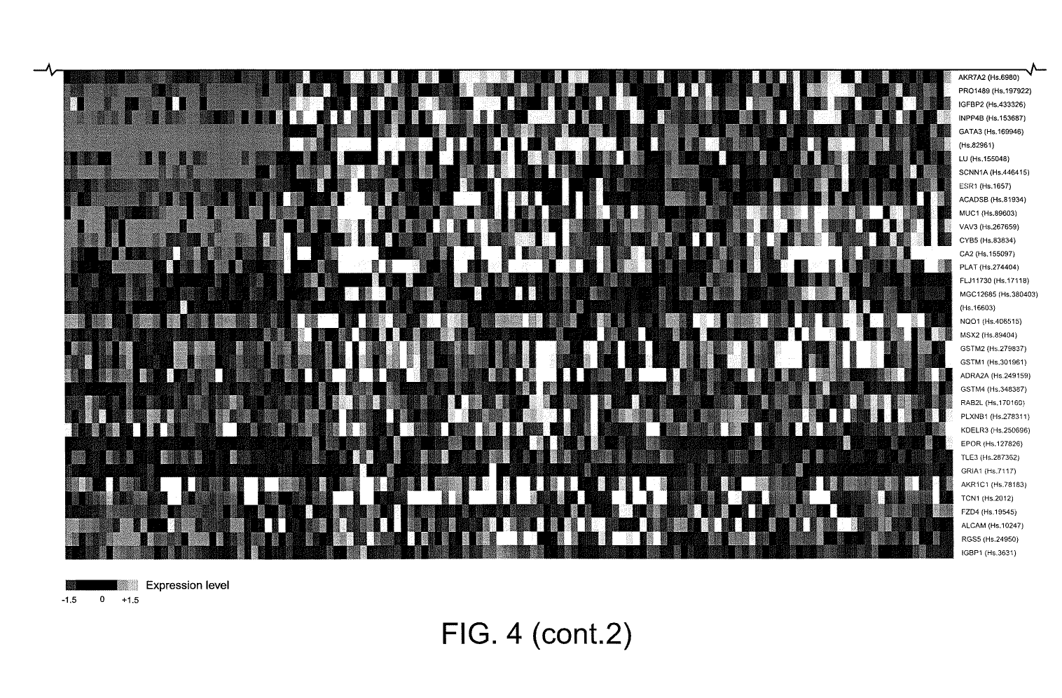

The differences in recurrent aberration composition between expression subtypes is consistent with a model of cancer progression in which the expression subtype and genotype are determined by the cell type and stage of differentiation that survives telomere crisis and acquires sufficient proliferative advantage to achieve clonal dominance in the tumor (Chin et al., 2004). This model indicates that the genome CNA spectrum is selected to be most advantageous to the progression of the specific cell type that achieves immortality and clonal dominance. In this model, the recurrent genome CNA composition can be considered an independent subtype descriptor—much as genome CNA composition can be considered to be a cancer type descriptor (Knuutila et al., 2000). The independence of the genome CNA composition and basal and luminal expression subtypes is clear from FIG. 4 which shows that the breast tumors divide into basal and luminal subtypes using unsupervised hierarchical clustering even after all transcripts showing associations with copy number are removed from the data set. Of course the ERBB2 subtype is lost since that subtype is strongly driven by ERBB2 amplification.

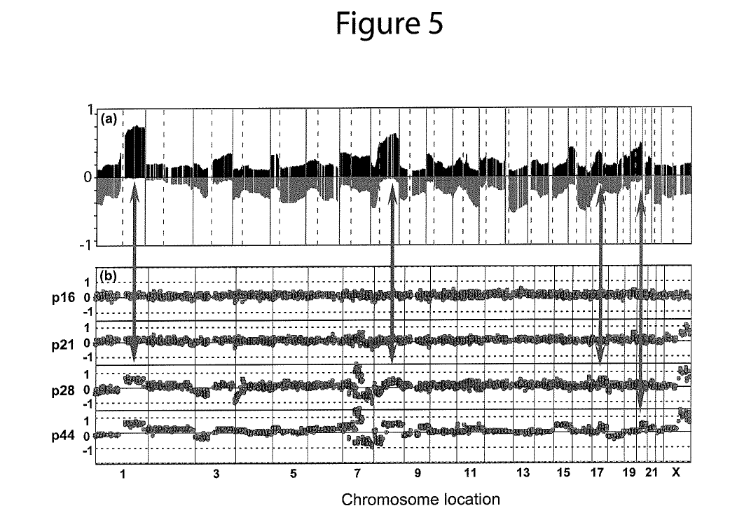

Low level abnormalities. The most frequent low-level copy number changes were not associated with reduced survival duration although some were associated with other markers usually associated with survival such as tumor size, nodal status, and grade (see Table 2). This raises the question of why the recurrent low-level CNAs are selected. To understand this, we applied the statistical tool GOstat to determine the ontology of the genes deregulated by these abnormalities. This analysis showed that numerous genes involved in RNA and cellular metabolism were significantly up-regulated by these events. Interestingly, we also observed that many of the recurrent low-level aberrations matched the low-level copy number changes in the ZNF217-transfected human mammary epithelial cells that emerged after passage through telomere crisis having achieved clonal dominance in the culture (see FIG. 5)—presumably because the aberrations they carried conferred a proliferative advantage (Chin et al., 2004). This indicates that the low-level CNAs contribute to early cancer formation by increasing basal metabolism thereby providing a net survival/proliferative advantage to the cells that carry them. This idea is supported by a report that some of these same classes of genes were associated with proliferative fitness yeast (Deutschbauer et al., 2005). That study described analyses of proliferative fitness in the complete set of Saccharomyces cerevisiae heterozygous deletion strains and reported reduced growth rates for strains carrying deletions in genes involved in RNA metabolism and ribosome biogenesis and assembly.

High level amplification. We found that high level amplifications of 8p11-12, 11q13-14, 17q12 and/or 20q13 were associated with reduced survival duration and/or distant recurrence overall, and within the luminal A expression subgroup. We identified 66 genes (see, e.g., Table 3) in these regions whose expression levels were correlated with copy number. These 66 genes are shown in Table 7 below along with the GenBank Accession numbers for each of the genes and gene products (proteins), the records of which are hereby incorporated by reference for all purposes. Also shown are the corresponding SEQ ID NOs as assigned here and shown in the sequence listing attached herein in computer readable form.

TABLE 7
Table 3 genes and their GenBank Accession and SEQ ID Numbers.

| | | DNA | Protein GenBank | PROTEIN |
|---|---|---|---|---|
| Gene | Genbank Accession No. | SEQ ID NO: | Accession Number | SEQ ID NO: |
| ACACA | NM_198839.1 | SEQ ID NO: 1 | NP_942136.1 | SEQ ID NO: 2 |
| | GI: 38679976 | | | |
| ADAM9 | AF495383 | SEQ ID NO: 3 | AAM49575.1 | SEQ ID NO: 4 |
| | var1: NM_003816; | SEQ ID NO: 5 | NP_003807.1 | SEQ ID NO: 6 |
| | var2: NM_001005845; | SEQ ID NO: 7 | NP_001005845.1 | SEQ ID NO: 8 |
| ERBB2 | AY208911; | SEQ ID NO: 9 | AAO18082.1; | SEQ ID NO: 10 |
| | NM_004448; | SEQ ID NO: 11 | NP_004439.2; | SEQ ID NO: 12 |
| | NM_001005862 | SEQ ID NO: 13 | NP_001005862.1 | SEQ ID NO: 14 |
| FGFR1 | AY585209 | SEQ ID NO: 15 | NP_075599.1 | SEQ ID NO: 16 |
| FNTA | NM_002027 | SEQ ID NO: 17 | NP_002018.1 | SEQ ID NO: 18 |
| IKBKB | AY663108; or | SEQ ID NO: 19 | AAT65965.1, or | SEQ ID NO: 20 |
| | NM_001556 XM_032491; | | NP_001547.1 | |
| NR1D1 | NM_021724 | SEQ ID NO: 21 | NP_068370.1 | SEQ ID NO: 22 |
| PNMT | NM_002686.3 | SEQ ID NO: 23 | NP_002677 | SEQ ID NO: 24 |
| PROSC | NM_007198 | SEQ ID NO: 25 | NP_009129.1 | SEQ ID NO: 26 |
| SPFH2 | AM393068 | SEQ ID NO: 27 | CAL37946.1 | SEQ ID NO: 28 |
| BRF2 | NM_018310 | SEQ ID NO: 29 | NP_060780.2 | SEQ ID NO: 30 |
| RAB11FIP1 | NM_001002814; | SEQ ID NO: 31 | NP_001002814.1 | SEQ ID NO: 32 |
| ASH2L | NM_004674; | SEQ ID NO: 33 | NP_004665.1 | SEQ ID NO: 34 |
| LSM1 | NM_014462; | SEQ ID NO: 35 | NP_055277.1 | SEQ ID NO: 36 |
| BAG4 | NM_004874 | SEQ ID NO: 37 | NP_004865.1 | SEQ ID NO: 38 |
| DDHD2 | NM_015214 XM_291291 | SEQ ID NO: 39 | NP_056029.1 | SEQ ID NO: 40 |
| WHSC1L1 | NM_023034 | SEQ ID NO: 41 | NP_075447.1 | SEQ ID NO: 42 |
| TACC1 | NM_206862 | SEQ ID NO: 43 | NP_996744.1 | SEQ ID NO: 44 |
| GOLGA7 | NM_016099 | SEQ ID NO: 45 | NP_057183.2 | SEQ ID NO: 46 |
| SLD5 | BC005995 | SEQ ID NO: 47 | AAH05995.1 | SEQ ID NO: 48 |
| MYST3 | NM_006766 | SEQ ID NO: 49 | NP_006757.1 | SEQ ID NO: 50 |
| AP3M2 | NM_006803 | SEQ ID NO: 51 | NP_006794.1 | SEQ ID NO: 52 |
| POLB | NM_002690 | SEQ ID NO: 53 | NP_002681.1 | SEQ ID NO: 54 |
| | | | AK018683 | |
| VDAC3 | NM_005662 | SEQ ID NO: 55 | NP_005653.3 | SEQ ID NO: 56 |
| SLC20A2 | NM_006749.3 | SEQ ID NO: 57 | NP_006740.1 | SEQ ID NO: 58 |
| THAP1 | NM_018105 | SEQ ID NO: 59 | NP_060575.1 | SEQ ID NO: 60 |
| LOC441347 | XM_940754 | SEQ ID NO: 61 | XP_945847.1 | SEQ ID NO: 62 |
| CCND1 | NM_053056 OR | SEQ ID NO: 63 | NP_444284.1 | SEQ ID NO: 64 |
| | NM_001758; | | | |
| FGF3 | NM_005247; | SEQ ID NO: 65 | NP_005238.1 | SEQ ID NO: 66 |
| FADD | CR456738 | SEQ ID NO: 67 | CAG33019.1 | SEQ ID NO: 68 |
| PPFIA1 | NM_003626; | SEQ ID NO: 69 | NP_003617.1 | SEQ ID NO: 70 |
| CTTN | NM_005231; | SEQ ID NO: 71 | NP_005222.2 | SEQ ID NO: 72 |
| NADSYN1 | NM_018161 | SEQ ID NO: 73 | NP_060631.2 | SEQ ID NO: 74 |
| KRTAP5-9 | NM_005553 | SEQ ID NO: 75 | NP_005544.4 | SEQ ID NO: 76 |
| FOLR3 | NM_000804 | SEQ ID NO: 77 | NP_000795.2 | SEQ ID NO: 78 |
| NEU3 | NM_006656.5 | SEQ ID NO: 79 | NP_006647.3 | SEQ ID NO: 80 |
| LHX1 | NM_005568.2 | SEQ ID NO: 81 | NP_005559.2 | SEQ ID NO: 82 |
| DDX52 | NM_007010.2 | SEQ ID NO: 83 | NP_008941.2 | SEQ ID NO: 84 |
| TBC1D3 | NM_032258.1 | SEQ ID NO: 85 | NP_115634.1 | SEQ ID NO: 86 |
| SOCS7 | NM_014598 XM_371052 | SEQ ID NO: 87 | NP_055413.1 | SEQ ID NO: 88 |
| PCGF2 | NM_007144.2 | SEQ ID NO: 89 | NP_009075.1 | SEQ ID NO: 90 |
| PSMB3 | NM_002795.2 | SEQ ID NO: 91 | NP_002786.2 | SEQ ID NO: 92 |
| PIP5K2B | NM_003559.4 | SEQ ID NO: 93 | NP_003550.1 | SEQ ID NO: 94 |
| FLJ20291 | AK000298.1 | SEQ ID NO: 95 | BAA91065 | SEQ ID NO: 96 |
| PPARBP | NM_004774.2 | SEQ ID NO: 97 | NP_004765.2 | SEQ ID NO: 98 |
| STARD3 | NM_006804.2 | SEQ ID NO: 99 | NP_006795.2 | SEQ ID NO: 100 |
| TCAP | NM_003673.2 | SEQ ID NO: 101 | NP_003664.1 | SEQ ID NO: 102 |
| PERLD1 | NM_033419 | SEQ ID NO: 103 | NP_219487.3 | SEQ ID NO: 104 |
| GRB7 | NM_001030002 | SEQ ID NO: 105 | NP_001025173.1 | SEQ ID NO: 106 |
| GSDML | NM_001042471; | SEQ ID NO: 107 | NP_001035936.1; | SEQ ID NO: 108 |
| | NM_018530 | SEQ ID NO: 109 | NP_061000.2 | SEQ ID NO: 110 |
| PSMD3 | NM_002809 | SEQ ID NO: 111 | NP_002800.2 | SEQ ID NO: 112 |
| ZNF217 | NM_006526 | SEQ ID NO: 113 | NP_006517.1 | SEQ ID NO: 114 |
| BCAS1 | NM_003657 | SEQ ID NO: 115 | NP_003648.1 | SEQ ID NO: 116 |
| CSTF1 | NM_001033521 | SEQ ID NO: 117 | NP_001028693.1 | SEQ ID NO: 118 |
| RAE1 | NM_003610 | SEQ ID NO: 119 | NP_003601.1 | SEQ ID NO: 120 |
| RNPC1 | NM_017495 | SEQ ID NO: 121 | NP_059965.2 | SEQ ID NO: 122 |
| PCK1 | AY794987 | SEQ ID NO: 123 | AAV50001.1 | SEQ ID NO: 124 |
| TMEPAI | NM_020182 | SEQ ID NO: 125 | NP_064567.2 | SEQ ID NO: 126 |
| RAB22A | NM_020673 | SEQ ID NO: 127 | NP_065724.1 | SEQ ID NO: 128 |
| VAPB | NM_004738 | SEQ ID NO: 129 | NP_004729.1 | SEQ ID NO: 130 |
| STX16 | NM_001001433 | SEQ ID NO: 131 | NP_001001433.1 | SEQ ID NO: 132 |
| NPEPL1 | NM_024663 or | SEQ ID NO: 133 | NP_078939.3 | SEQ ID NO: 134 |
| | NM_207402 | | | |
| GNAS | NM_000516 | SEQ ID NO: 135 | NP_000507.1 | SEQ ID NO: 136 |
| TH1L | NM_198976 | SEQ ID NO: 137 | NP_945327.1 | SEQ ID NO: 138 |
| N-PAC | NM_032569 NM_018459 | SEQ ID NO: 139 | NP_115958.2 | SEQ ID NO: 140 |
| C20orf45 | NR_003259 | SEQ ID NO: 141 | | |

GO analyses of those genes showed that they are involved in aspects of nucleic acid metabolism, protein modification, signaling and the cell cycle and/or protein transport and evidence is mounting that many if not most of these genes are functionally important in the cancers in which they are amplified and over expressed (see Table 3). Indeed, published functional studies in model systems already have implicated fourteen genes in diverse aspects of cancer pathophysiology (Table 3, column 8).

Six of these are encoded in the region of amplification at 8p11. These are the RNA binding protein, LSM1 (GenBank Accession No. NM_{—}014462; SEQ ID NO:35; Fraser et al., 2005), the receptor tyrosine kinase, FGFR1 (GenBank Accession No. AY585209; SEQ ID NO: 15; Braun and Shannon, 2004), the cell cycle regulatory protein, TACC1 (GenBank Accession No. NM_{—}206862; SEQ ID NO: 43; Still et al., 1999), the metalloproteinase, ADAM9 (GenBank Accession Nos. AF495383, NM_{—}003816, NM_{—}001005845; SEQ ID NOs: 3, 5, and 7; Mazzocca et al., 2005), the serine/threonine kinase, IKBKB (GenBank Accession Nos. AY663108, NM_{—}001556, XM_{—}032491; SEQ ID NO: 19; Greten and Karin, 2004; Lam et al., 2005) and the DNA polymerase, POLB (GenBank Accession No. NM_{—}002690, SEQ ID NO: 53; Clairmont et al., 1999).

Functionally validated genes in the region of amplification at 11q13 include the cell cycle regulatory protein, CCND1 (GenBank Accession Nos. NM_{—}053056, NM_{—}001758; SEQ ID NO: 63; Hinds et al., 1994), and the growth factor, FGF3 (GenBank Accession Nos. NM_{—}005247, SEQ ID NO: 65; Okunieff et al., 2003).

Functionally important genes in the region of amplification at 17q include the transcription regulation protein, PPARBP (GenBank Accession No. NM_{—}004774.2; SEQ ID NO: 97; Zhu et al., 2000), the receptor tyrosine kinase ERBB2 (GenBank Accession No. AY208911, NM_{—}004448, NM_{—}001005862; SEQ ID NOs: 9, 11, 13; Slamon et al., 1989) and the adapter protein, GRB7 (GenBank Accession No. NM_{—}001030002, SEQ ID NO: 105; Tanaka et al., 2000).

The AKT pathway-associated-transcription factor, ZNF217 (GenBank Accession No. NM_{—}006526; SEQ ID NO: 113; Huang et al., 2005; Nonet et al., 2001) and the RNA binding protein, RAE1 (GenBank Accession No. NM_{—}003610; SEQ ID NO: 119; Babu et al., 2003) are functionally validated genes encoded in the region of amplification at 20q13.

As set forth in Table 3, column 9, further support for the functional importance of 21 of these genes (TACC1, ADAM9, IKBKB, POLB, CCND1, PCGF2, PSMB3, PIP5K2B, FLJ20291, STARD3, TCAP, PNMT, PERLD1, GRB7, GSDML, PSMD3, NR1D1, ZNF217, BCAS1, TH1L, and C20orf45) in oncogenesis comes from the observation that they are within 100 Kbp of sites of recurrent tumorigenic viral integration in the mouse (Akagi et al., 2004); in particular, three (IKBKB, CCND1, GRB7) are within 10 Kbp of such a site. Taking proximity to a site of recurrent tumorigenic viral integration as evidence for a role in cancer genesis, an additional 13 genes or transcripts are implicated (see Table 3); these are the genes that are near viral insertion sites but are: (1) not associated with outcome [highlighted gray] and (2) not previously associated to cancer [column 8].

The biological roles of the genes deregulated by recurrent high level amplification are diverse and vary between regions of amplification. For example, genes deregulated by amplification at 11q13 and 17q11-12 predominantly involved signaling and cell cycle regulation while genes deregulated by amplification at 8p11-12 and 20q13 were of mixed function but were associated most frequently with aspects of nucleic acid metabolism. The predominance of genes involved in nucleic acid metabolism in the region of amplification at 8p11-12 was especially strong.

Gene Deletion. Interestingly, the region of recurrent amplification at 8p11-12 described above was reduced in copy number in some tumors and this event also was associated with poor outcome. Thus, this is evidence that the poor clinical outcome in tumors with 8p11-12 abnormalities is due to increased genome instability/mutagenesis resulting from either up- or down-regulation of genes encoded in this region. This is supported by studies in yeast showing that up- or down-regulation of genes involved in chromosome integrity and segregation can produce similar instability phenotypes (Ouspenski et al., 1999).

### Therapeutic Targets

Thus, the 66 genes we set forth in Table 3 were found to be deregulated by the high level amplifications and were associated with poor outcome; these genes and their gene products serve as therapeutic targets for cancer treatment, in particular those patients that are refractory to current therapies. Small molecule or antibody based inhibitors have already been developed for FGFR1 (PD173074, (Ray et al., 2004)), IKBKB (PS-1145; (Lam et al., 2005)) and ERBB2 (Trastuzumab, (Vogel et al., 2002)).

Six genes set forth in Table 3 (PROSC, ADAM9, FNTA, ACACA, PNMT, and NR1D1) are considered as druggable based on the presence of predicted protein folds that favor interactions with drug-like compounds (Russ and Lampel, 2005).

Taking ERBB2 as the paradigm (recurrently amplified, over expressed, associated with outcome and with demonstrated functional importance in cancer), indicates that FGFR1, TACC1, ADAM9, IKBKB, PNMT, and GRB7 are high priority therapeutic targets in these regions of amplification. Thus, it is expected that the studies and effects of inhibition on ADAM9, as described in Example 10, may be carried out and observed for any of these genes as well. Furthermore, it is contemplated that antagonists of these genes can be made by one having skill in the art, including but not limited to, inhibitory oligonucleotides and peptides, aptamers, small molecules, drugs and antibodies, thereby producing an effect on the gene or gene product as a treatment for breast cancer.

### Molecular Characteristics and Associations.

We assessed genome copy number using BAC array CGH (Hodgson et al., 2001; Pinkel et al., 1998; Snijders et al., 2001; Solinas-Toldo et al., 1997) and gene expression profiles using Affymetrix U133A arrays (Ramaswamy et al., 2003; Reyal et al., 2005) in breast tumors from a cohort of patients treated according to the standard of care between 1989 and 1997 (surgery, radiation, hormonal therapy and treatment with high dose adriamycin and cytoxan as indicated). We measured genome copy number profiles for 145 primary breast tumors and gene expression profiles for 130 primary tumors, of which 101 were in common. We analyzed these data to identify recurrent genomic and transcriptional abnormalities and we assessed associations with clinical endpoints to identify genomic events that might contribute to cancer pathophysiology.

Genome copy number and gene expression features. We found that the recurrent genome copy number and gene expression characteristics measured for the patient cohort in this study were similar to those reported in earlier studies. We summarize these briefly.

FIG. 1(a) and FIG. 1(b) show numerous regions of recurrent genome CNA and 9 regions, as shown in Table 1, of recurrent high level amplification involving regions of chromosomes 8, 11, 12, 17 and 20 while FIG. 2 shows that analysis of these data using unsupervised hierarchical clustering resolves these tumors into the “1q/16q” (or “simple”), “complex” and “amplifier” genome aberration subtypes (Fridlyand et al., 2006). The genomic extents of the regions of amplification are listed in Table 1. These were generally similar to those reported in earlier studies using chromosome (Kallioniemi et al., 1994) and array CGH (Loo et al., 2004; Naylor et al., 2005; Pollack et al., 1999; Pollack et al., 2002). Several of these regions of amplification were frequently co-amplified. Declaring a Fisher exact test p-value of less than 0.05 for pair-wise associations to be suggestive of possible significant co-amplification, we found co-amplification of 8q24 and 20q13 and co-amplification of regions at 11q13-14, 12q13-14, 17q11-12, and 17q21-24. These analyses were underpowered to achieve significance with proper correction for multiple testing so these associations are suggestive but not significant. However, these associations were consistent with the report of Al Kuraya et al (Al-Kuraya et al., 2004) who showed evidence for co-amplification of genes in several of these regions of amplification including ERBB2, MYC, CCND1 and MDM2 and that of Naylor et al (Naylor et al., 2005) showing co-amplification of 17q12 and 17q25.

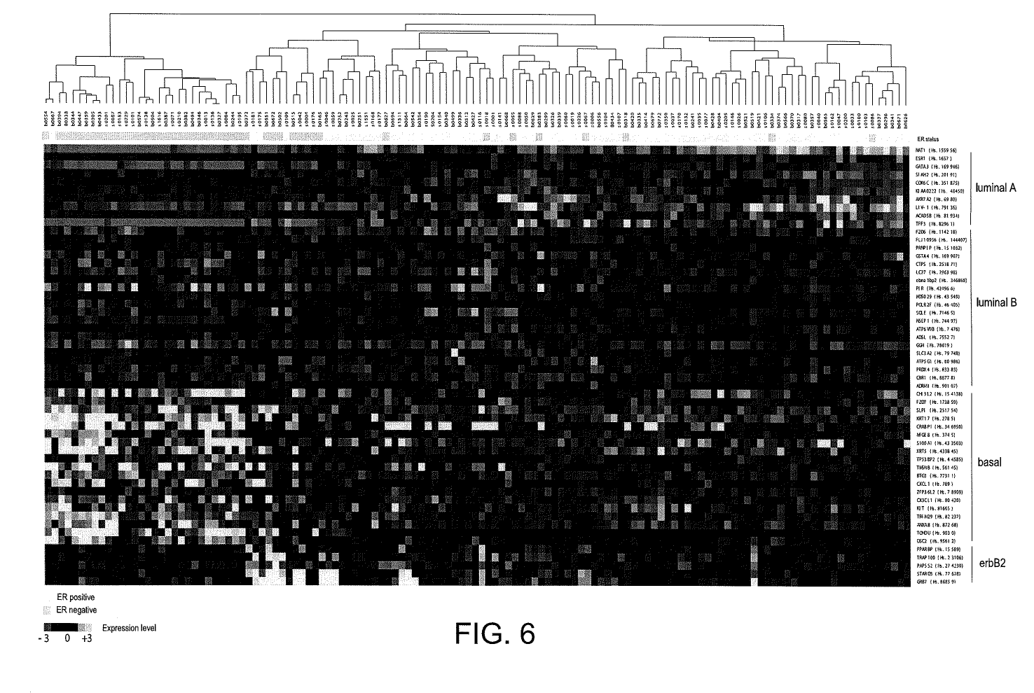

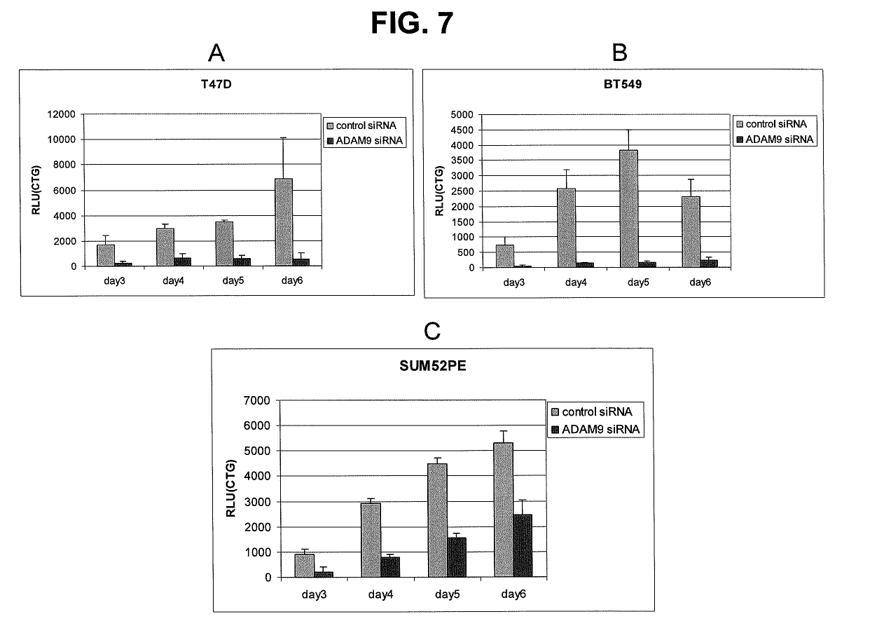

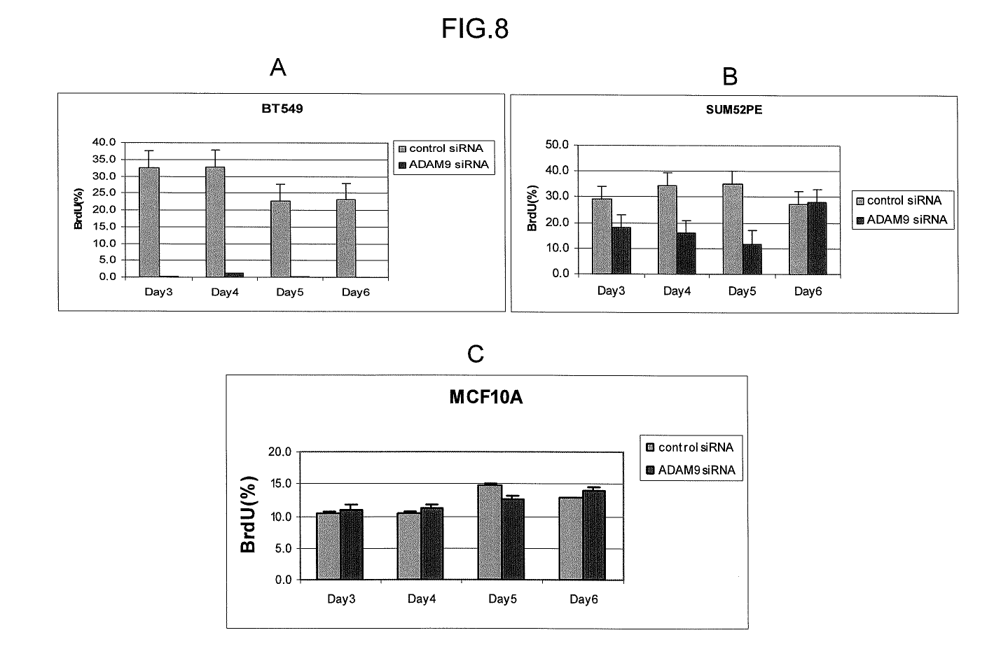

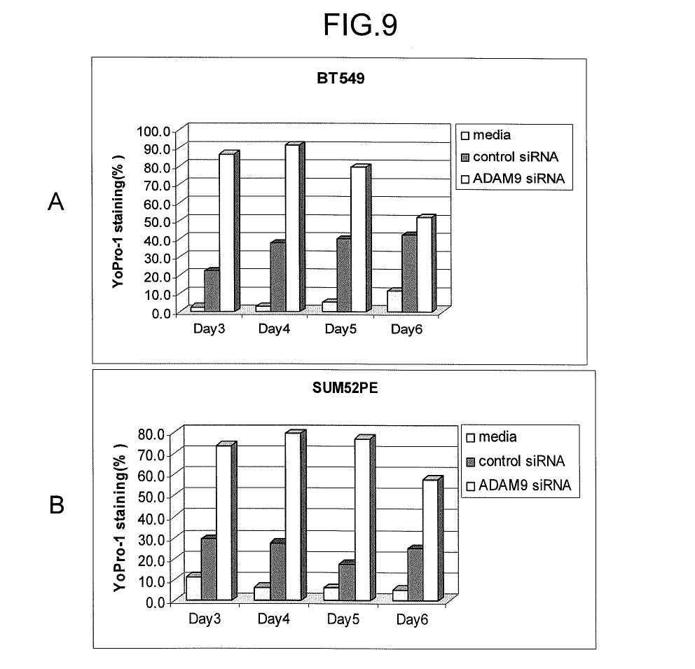

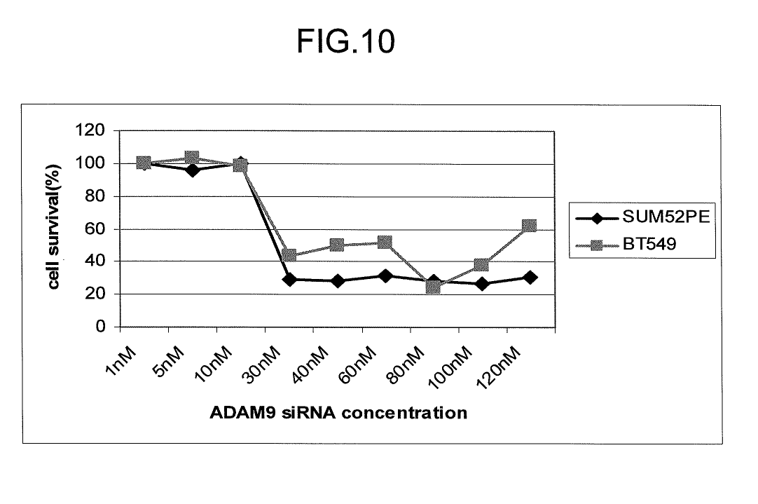

FIG. 6 shows that unsupervised hierarchical clustering of intrinsically variable genes resolves the tumors in our study cohort into the luminal A, luminal B, basal-like and ERBB2 expression subtypes previously reported for breast tumors (Perou et al., 1999; Perou et al., 2000; Sorlie et al., 2003). We assessed the genomic characteristics of these expression subtypes in subsequent analyses.

Associations between CNAs and expression. Combined analyses of genome copy number and expression showed that the recurrent genome CNAs differed between expression subtypes and identified genes whose expression levels were significantly deregulated by the CNAs. FIGS. 1(c)-1(j) show the recurrent CNAs for each expression subtype. In these analyses, we assigned each tumor to the expression subtype cluster (basal-like, ERBB2, luminal A, and luminal B) to which its expression profile was most highly correlated. We did not assess aberration in normal-like tumors due to the small number of such tumors.

FIG. 1(c) shows that the basal-like tumors were relatively enriched for low-level copy number gains involving 3q, 8q, and 10p and losses involving 3p, 4p, 4q, 5q, 12q, 13q, 14q and 15q while FIG. 1(d) shows that high level amplification at any locus was infrequent in these tumors. FIG. 1(e) shows that ERBB2 tumors were relatively enriched for increased copy number at 1q, 7p, 8q, 16p and 20q and reduced copy number at 1p, 8p, 13q and 18q. FIG. 1(f) shows that amplification of ERBB2 was highest in the ERBB2 subtype as expected but amplification of noncontiguous, distal regions of 17q also was frequent as previously reported (Barlund et al., 1997). FIG. 1(g) shows that increased copy number at 1q and 16p and reduced copy number at 16q were the most frequent abnormalities in luminal A tumors while FIG. 1(h) shows that amplifications at 8p11-12, 11q13-14, 12q13-14, 17q11-12, 17q21-24 and 20q13 were relatively common in this subtype. FIG. 1(i) shows that gains of chromosomes 1q, 8q, 17q and 20q and losses involving portions of 1p, 8p, 13q, 16q, 17p and 22q were prevalent in luminal B tumors while FIG. 1(j) shows that high level amplifications involving 8p11-12, two regions of 8q, and 11q13-14 were frequent.

In order to understand how the genome aberrations were influencing cancer pathophysiologies, we identified genes that were deregulated by recurrent genome CNAs. We took these genes to be those whose expression levels were significantly associated with copy number (Holm-adjusted p-value <0.05). These genes, which represent about 10% of the genome interrogated by the Affymetrix HGU133A arrays used in this study, and their copy number-expression level correlation coefficients are listed in Table 4 This extent of genome-aberration-driven deregulation of gene expression is similar to that reported in earlier studies (Hyman et al., 2002; Pollack et al., 1999).

We tested associations between copy number and expression level for 186 genes in regions of amplification at 8p11-12, 11q13-q14, 17q11-12 and 20q13 (see Table 5) and we identified 66 genes in these regions whose expression levels were correlated with copy number (FDR<0.01, wilcoxon rank sum test; Table 3). These genes define the transcriptionally important extents of the regions of recurrent amplification. Twenty-three were from a 5.5 Mbp region at 8p11-12 flanked by SPFH2 and LOC441347, ten were from a 6.6 Mbp region at 11q13-14 flanked by CCND1 and PRKRIR, nineteen were from a 3.1 Mbp region at 17q12 flanked by LHX1 and NR1D1 and fourteen were from a 5.4 Mbp region at 20q13 flanked by ZNF217 and C20orf45.

Since the recurrent genome aberrations differed between expression subtypes, we explored the extent to which the expression subtypes were determined by genome copy number. Specifically, we applied unsupervised hierarchical clustering to intrinsically variable genes after removing genes whose expression levels were correlated with copy number. FIG. 4 shows that the tumors still resolve into the basal-like and luminal classes. However, the ERBB2 cluster was lost.

Associations with Clinical Variables.

Associations with histopathology. FIG. 2 and Table 2 summarize associations of histopathological features with aspects of genome abnormality including recurrent genome abnormalities, total number of copy number transitions, fraction of the genome altered (FGA), number of chromosomal arms containing at least one amplification, number of recurrent amplicons and presence of at least one recurrent amplification.

These analyses showed that ER/PR negative tumors were predominantly found in the basal-like and “complex” expression and genome aberration subtypes, respectively. Node-positive tumors had significantly more amplified arms and recurrent amplicons than node-negative samples but showed a much more moderate difference in terms of low-level copy number transitions. Stage 1 tumors had moderately fewer low- and high-level changes than higher stage tumors. The number of low and high level abnormalities increased with SBR grade. Interestingly, the “complex” tumors showing many low-level abnormalities were more strongly associated with aberrant p53 expression than “amplifying” tumors. “Simple” tumors tended to have Ki67 proliferation indices<10% while “complex” and “amplifying” tumors typically had Ki67 indices>10%. The number of amplifications increased significantly with tumor size but the number of low level changes did not. We observed no association of genomic changes with the age at diagnosis.

Associations with outcome. FIG. 4 and Table 5 summarize associations between histopathological, transcriptional and genomic characteristics and outcome endpoints identified using multivariate regression analysis. Histopathological features including size and nodal status were significantly associated with survival duration and/or disease recurrence in univariate analyses (Table 4) and were included in the multivariate regressions described below.

The tumor subtypes based on patterns of gene expression or genome aberration content showed moderate associations with outcome endpoints. For example, FIG. 3(a) shows that patients with tumors classified as ERBB2 based on expression pattern had significantly shorter disease-specific survival than patients classified as luminal A, luminal B, or normal-like as previously reported (Perou et al., 2000; Sorlie et al., 2001). Unlike these earlier reports, patients with tumors classified as basal-like did not do significantly worse than patients with luminal or normal breast-like tumors although there was a trend in that direction. In addition, FIG. 3(b) indicates that patients with tumors classified as “1q/16q” based on genome aberration content tended to have longer disease-specific survival than patients with “complex” or “amplifier” tumors.

We found that high level amplification was most strongly associated with poor outcome in this aggressively treated patient population. Amplification at any of the 9 recurrent amplicons was an independent risk factor for reduced survival duration (p<0.04) and distant recurrence (p<0.01) in a multivariate Cox-proportional model that included tumor size and nodal status. FIG. 3(c), for example, shows that patients whose tumors had at least one recurrent amplicon survived a significantly shorter time than did patients with tumors showing no amplifications. More specifically, amplifications of 8p11-12 or 17q11-12 (ERBB2) were significantly associated with disease-specific survival and distant recurrence in all patients in multivariate regressions (Table 1).

Importantly, we found that stratification according to amplification status allowed identification of patients with poor outcome even within an expression subtype. FIG. 3(d), for example, shows that patients with luminal A tumors and amplification at 8p11-12, 11q13-14 or 20q13 had significantly shorter disease-specific survival than patients without amplification in one of these regions (the number of samples in the luminal A subtype group was too small for multivariate regressions). Amplification at 8p11-12 was most strongly associated with distant recurrence in the luminal A subtype.

Considering the strong association between amplification and outcome, we explored the possibility that some of these genes were over expressed in tumors in which they were not amplified and that over expression was associated with reduced survival duration in those tumors. Increased expression levels of 7 genes are labeled in Table 3 in dark gray (CTTN, KRTAP5-9, LHX1, PPARBP, PNMT, GRB7, TMEPAI). These genes were associated with reduced survival or distant recurrence at the p<0.1 level but only two, the growth factor receptor binding protein, GRB7 (17q) and the keratin associated protein, KTRAP5-9 (11q), at the p<0.05 level.

Interestingly, this expression analysis also revealed an unexpected association between reduced expression levels of genes from regions of amplification and poor outcome (either disease free survival or distant recurrence) in tumors without relevant amplifications (p<0.05). This was especially prominent for genes from the region of amplification at 8p11-12 (14 of 23 genes in this region showed this association) while only two genes from regions of adverse-outcome-associated amplifications on chromosomes 17q and 20q showed this association.

Following this lead, we tested associations between outcome and reduced copy number at 8p11-12 in patients in tumors in which 8p11-12 was not amplified. FIG. 3(e) shows that patients with reduced copy number at 8p11-12 did worse than patients without a deletion in this region. FIG. 3(f) shows that patients in the overall study with high level amplification or deletion at 8p11-12 survived significantly shorter survival (p=0.0017) than patients without either of those events.

We also tested for associations of low level genome copy number changes with the outcome endpoints. The most frequent low-level copy number changes (e.g. increased copy number at 1q, 8q and 20q or decreased copy number at 16q) were not significantly associated with outcome endpoints. However, we did find a significant association of the loss of a small region on 9q22 with adverse outcome, both disease-specific survival and distal recurrence, which persisted even after correction for multiple testing (p<0.05, multivariate Cox regression). This region is defined by BACs, CTB-172A10 and RP11-80F13. We also found a marginally significant association between fraction of the genome lost and disease-specific survival in luminal A tumors (p<0.02 and <0.06 for univariate and multivariate regression, respectively, Wilcoxon rank-sum test).

The lack of association of the most frequent low level CNAs with outcome raised the issue of selection pressure during tumor evolution. To understand this, we used the program GoStat (Beissbarth and Speed, 2004) to identify the Gene Ontology (GO) classes of 1444 unique genes (1734 probe sets) whose expression levels were preferentially modulated by low-level CNAs compared to 3026 probe sets whose expression levels did not show associations with copy number. The GO categories most significantly overrepresented in the set of genes with a dosage effect compared to genes with no or minimal dosage effect involved RNA processing (Holm adjusted p-value<0.001), RNA metabolism (p<0.01) and cellular metabolism (p<0.02).

### EXAMPLES

### Example 1

### Tumor Characteristics

Frozen tissue from UC San Francisco and the California Pacific Medical Center collected between 1989 and 1997 was used for this study. Tissues were collected under IRB approved protocols with patient consent. Tissues were collected, frozen over dry ice within 20 minutes of resection, and stored at −80° C. An H&E section of each tumor sample was reviewed, and the frozen block was manually trimmed to remove normal and necrotic tissue from the periphery. Clinical follow-up was available with a median time of 6.6 years overall and 8 years for censored patients. Tumors were predominantly early stage (83% stage I & II) with an average diameter of 2.6 cm. About half of the tumors were node positive, 67% were estrogen receptor positive, 60% received tamoxifen and half received adjuvant chemotherapy (typically adriamycin and cytoxan). Clinical characteristics of the individual tumors are provided together with expression and array CGH profiles in the CaBIG repository and at http://graylabdata.lbl.gov.

### Example 2

### Array CGH

Each sample, such as from Example 1, was analyzed using Scanning and OncoBAC arrays. Scanning arrays were comprised of 2464 BACs selected at approximately megabase intervals along the genome as described previously (Hodgson et al., 2001; Snijders et al., 2001). OncoBAC arrays were comprised of 1860 PI, PAC, or BAC clones. About three-quarters of the clones on the OncoBAC arrays contained genes and STSs implicated in cancer development or progression. All clones were printed in quadruplicate. DNA samples for array CGH were labeled generally as described previously (Hackett et al., 2003; Hodgson et al., 2001; Snijders et al., 2001). Briefly, 500 ng each of cancer and normal female genomic DNA sample was labeled by random priming with CY3- and CY5-dUTP, respectively; denatured; and hybridized with unlabeled Cot-1 DNA to CGH arrays. After hybridization, the slides were washed and imaged using a 16-bit CCD camera through CY3, CY5, and DAPI filters (Pinkel et al., 1998).

Statistical considerations. Data processing. Array CGH data image analyses were performed as described previously (Jain et al., 2002). In this process, an array probe was assigned a missing value for an array if there were fewer than 2 valid replicates or the standard deviation of the replicates exceeded 0.2. Array probes missing in more than 50% of samples in OncoBAC or Scanning array datasets were excluded in the corresponding set. Array probes representing the same DNA sequence were averaged within each dataset and then between the two datasets. Finally, the two datasets were combined and the array probes missing in more than 25% of the samples, unmapped array probes and probes mapped to chromosome Y were eliminated. The final dataset contained 2149 unique probes.

### Example 3

### Expression Profiling Using the Affymetrix High Throughput Analysis (HTA) System

Expression array analysis using the GeneChip® assay is implemented on the Affymetrix HTA system in four automated procedures; target preparation, hybridization, washing/staining and scanning.

Target preparation. For each sample, the RNA target is prepared by putting 2.5 g of total RNA in 5 μl water and 5 μl of 10 μM T7(dt)24 primer into a MJ Research 96-well reaction plate. The total RNA undergoes an annealing step at 70° C. for 10 minutes followed by a 4° C. cooling step for 5 minutes. The plate is transferred back to the deck position and undergoes first strand cDNA synthesis. 10 μl of First Strand Cdna Synthesis cocktail (4 μl of Affymetrix 5×1 st strand buffer (250 mM Tris-HCl, pH 8.3 at room temperature; 375 mM KCl; 15 mM MgCl2), is mixed with 2 μl 0.1M DTT, 1 μl 10 mM dNTP mix, 1 μl Superscript II (200U/ul), and 2 μl nuclease free water per reaction) is added, and the plate is then transferred to the thermal cycler and incubated at 42° C. for 60 minutes and 4° C. for 5 min. 91 μl of nuclease free water and 39 μl of the Second Strand cDNA Synthesis cocktail (30 μl of Affymetrix 5×2nd strand buffer, 100 mM Tris-HCl (pH 6.9), 23 mM MgCl2, 450 mM KCl, 0.75 mM β-NAD, 50 mM (NH4)2SO4); 3 μl 10 mM dNTP; 1 μl 10 unit/μl DNA Ligase; 4 μl 10 unit/μl DNA Polymerase and 1 μl 2 units/μl RNase H) is added. The plate is incubated at 16° C. for 120 minutes and 4° C. for 5 minutes. 4 μl of T4 Polymerase cocktail comprised of 2 μl T4 DNA Polymerase plus 2 μl 1× T4 DNA Polymerase Buffer (165 mM Tris-acetate (pH 7.9), 330 mM Sodium-acetate, 50 mM Magnesium-acetate, 5 mM DTT) is added and the plate is taken back to the thermal cycler where it is cycled at 16° C. for 10 minutes, 72° C. for 10 minutes, and cooled to 4° C. for 5 minutes.

The plate is transferred back to the deck and Agencourt Magnetic Beads are used for the cDNA clean-up. 162 μl of magnetic beads are mixed with 90 μl of in the cDNA Clean-Up Plate and incubated for 5 minutes. Post incubation, the cDNA bound to the beads in the cDNA Clean-Up Plate is moved to the Agencourt magnetic plate. Another 115 μl of magnetic beads is mixed with 64 μl cDNA incubated for 5 minutes, and then moved to the Agencourt magnetic plate. Post incubation, the supernatant is removed and two washes with 75% EtOH are performed using 200 μl solution. The EtOH is then removed and the beads sit for 5 minutes. 40 μl of nuclease free water is added to the beads and mixed well. The solution is then incubated for 1 minute, and then it is taken back to the magnetic plate where it is incubated for 5 minutes to capture the beads on the magnet. 22 μl of eluted cDNA is then transferred to the Purified cDNA Plate (22 μl total volume). 38 μl of IVT cocktail (6 μl 10× IVT Buffer, 18 μl HTA RLR Reagent (labeling NTP), 6 μl HTA Enzyme Mix, 1 μl T7 RNA Polymerase, and 7 μl RNase free water per reaction is added to the purified cDNA) is added to the 22 μl of purified cDNA (60 μl total volume). The plate is then transferred to the thermal cycler where incubation of 8 hours at 37° C. occurs.

Upon completion, the plate is transferred back to the deck where 120 μL Agencourt Magnetic Beads are used to clean up the cRNA product. The A260 of the purified cRNA is measured in a plate spectrophotometer, then the concentration in each well of a 96 well plate is adjusted to a calculated value of 0.625 μg/μl. A second reading is taken to verify the normalization process. 30 μl of cRNA was transferred from the cRNA Normalization Plate and dispensed in the Fragmented cRNA Plate. 7.5 μl of 5× fragmentation buffer per sample is added. The plate is then transferred to the thermal cycler where it is held at 94° C. for 35 minutes followed by a cooling step at 20° C. for 5 minutes. The sample is then mixed with 90 μl of hybridization cocktail (3 μl of 20× bioB, bioC, bioD, and creX hybridization controls mixed with 1.6 μl 3 nM oligo-B2, 1 μl 10 mg/ml Herring sperm DNA, 1 μl 50 mg/ml acetylated BSA, and 83.4 μl 1.2× Hybridization Buffer).

Hybridization. The sample is then ready to be hybridized. The peg array plate is incubated in 60 μl pre-hybridization cocktail (1 μl 10 mg/ml Herring sperm DNA, 1 μl 150 mg/ml Acetylated BSA, 84 μl Hybridization buffer, 15 μl nuclease free H20 per reaction). The hybridization-ready sample is taken to the thermal cycler and denatured for 95° C. for 5 minutes. Upon completion of this step, the plate is returned to the deck where 70 μl of sample is transferred to a hybridization tray. The peg plate is then lifted off of the pre-hybridization tray and taken to the hybridization plate where it is placed. This “hybridization sandwich” is then manually transferred to a hybridization oven where it incubates at 48° C. for 16-18 hours. Washing/Staining. The robot lifts the peg plate off of the hybridization tray and transfers it to the first low stringency wash (LSW) (6×SSPE, 0.01% Tween-20) where it is dipwashed 36 times. The plate is then transferred to the other three low stringency wash positions where the dipping is repeated. The peg plate is then moved to the high stringency wash (HSW) (100 mM MES, 0.1M NaCl, 0.01% Tween-20) where it is incubated at 41° C. for 25 minutes. After the incubation, the peg plate is transferred to a fifth LSW tray where the HSW removed by rinsing. The plate is transferred to the first stain (31.5 μl nuclease free H20, 35 μl 2×MES stain buffer, 2.8 μl 50 mg/ml Acetylated BSA, 0.7 μl R-Phycoerythrin Streptavidin), where it incubates at room temperature for 10 minutes. At the end of the 10 minute incubation, the peg plate undergoes another 4 cycles of dip washing method. The peg tray is then transferred to stain 2 (2.8 μl 50 mg/ml Acetylated BSA, 0.7 μl reagent grade goat IgG, 0.4 μl biotinylated goat Anti-streptavidin antibody per reaction). The above method is repeated for stain 3 (31.5 μl nuclease free H20, 35 μl 2×MES stain buffer, 2.8 μl 50 mg/ml Acetylated BSA, 0.7 μl R-Phycoerythrin Streptavidin). At the end of the incubation of the third stain, the peg plate is washed 36 times in LSW. The robot then transfers 70 μl of MES holding buffer, 68 mM MES, 1.0 M NaCl, 0.01% Tween-20, into a sterile scan tray. The peg tray is then placed into the scan tray for scanning.

Scanning. The 96 well peg plate is scanned by the Affymetrix High Throughput (HT) scanner, a fully automated epi-fluorescence imaging system with an excitation wavelength range of 340 nm to 675 nm and a cooled 1280×1024 CCD camera with 12 bit readout. Scanning resolution is 1.0 μm/pixel with a 10× objective. Images are captured at two different exposure times. Each well will have 49 sub-images/exposure times. The software program then converts these .dat files into mini .cel files and then into composite cel files where the information is analyzed in the Affymetrix GCOS 1.2 software.

Statistical considerations. Data processing. For Affymetrix data, multi-chip robust normalization was performed using RMA software (Irizarry et al., 2003). Transcripts assessed on the arrays were classified into two groups using Gaussian model-based clustering by considering the joint distribution of the median and standard deviation of each probe set across samples. During this process, computational demands were reduced by randomly sampling and clustering 2000 probe intensities using melust (Yeung et al., 2001; Yeung et al., 2004) with two clusters and unequal variance. Next, the remaining probe intensities were classified into the newly created clusters using linear discriminant analysis. The cluster containing probe intensities with smaller mean and variance was defined as “not expressed” and the second cluster was “expressed”.

### Example 4

### Assessment of Genome Copy Numbers

Characterizing copy number changes. The array CGH data were analyzed using circular binary segmentation (CBS) (Olshen et al., 2004) to translate intensity measurements into regions of equal copy number as implemented in the DNA copy R/Bioconductor package. Missing values for probes mapping within segmented regions of equal copy number were imputed by using the value of the corresponding segment. A few probes with missing values (<0.3%) were located between segmented regions and their values were imputed using the maximum value of the two flanking segments. Thus, each probe was assigned a segment value referred to as its “smoothed” value. The scaled median absolute deviation (MAD) of the difference between the observed and smoothed values was used to estimate the tumor-specific experimental variation. All tumors had noise standard deviation of less than 0.2. The gain and loss status for each probe was assigned using the merge Level procedure as described (Willenbrock and Fridlyand, 2005). In this process, segmental values across the genome were merged to create a common set of copy number levels for each individual tumor. The probes corresponding to the copy number level with the smallest absolute median value were declared unchanged whereas all the other probes were either gained or lost depending on the sign of the segment mean. Additionally, to account for high level focal aberrations being single outliers and thus assigned the status of the surrounding segments, the probe was assigned gain status when amplified as described below.

The frequency of alterations at each probe locus was computed as the proportion of samples showing an aberration at that locus. The genome distance assigned to each probe was computed by assigning a genomic distance equal to half the distance to the neighboring probes or to the end of a chromosome for the probes with only one neighbor. The number of copy number transitions was computed based on the initial DNA copy segmentation by counting the number of copy number transitions in the genome (Snijders et al., 2003). Single outliers such as high level amplifications were identified by assigning the original observed log2ratio to the probes for which the observed values were more than 4 tumor-specific MAD away from the smoothed values. The amplification status for a probe was then determined by considering the width of the segment to which that probe belonged (0 if an outlier) and a minimum difference between the smoothed value of the probe (observed value if an outlier) and the segment means of the neighboring segments. The clone was declared amplified if it belonged to the segment spanning less than 20 Mb and the minimum difference was greater than exp(−x³) where x is the final smoothed value for the clone. Note that this allowed clones with small log2ratio to be declared amplified if they were high relative to the surrounding clones with the required difference becoming larger as value of the clone gets smaller (e.g. a difference of 1 was required when clone value was 0 and 0.36 when the clone value was 1; Albertson, Fridlyand, private communication).

Clustering of genome copy number profiles. Genome copy number profiles were clustered using smoothed imputed data with outliers present. Agglomerative hierarchical clustering with Pearson correlation as a similarity measure and the Ward method to minimize sum of variances were used to produce compact spherical clusters (Hartigan, 1975). The number of groups was assessed qualitatively by considering the shape of the clustering dendogram.

Expression subtype assignment. Tumors were classified according to expression phenotype (basal, ERBB2, luminal A, luminal B and normal-like) by assigning each tumor to the subtype of the cluster defined by hierarchical clustering of expression profiles for 122 samples published by Sorlie et al (Sorlie et al., 2003) to which it had the highest Pearson correlation. The correlation was computed using the subset of Stanford intrinsically variable genes common to both datasets. Unigene IDs were used to match the probes and genes with non-unique Unigene IDs. These data were averaged and the genes were median-centered for both datasets. For robustness, only 79 of the most tightly clustered Stanford samples were used to define Stanford cluster centroids. Unigene IDs for Affymetrix data were obtained from the TIGR Resourcer website, http://pga.tigr.org/tigr-scripts/magic/rl.pl. The Stanford intrinsic genes list was downloaded from http://genome-www.stanford.edu/breast_cancer/robustness/data.shtml. The same procedure was used to assign expression subtypes to the 295 breast tumors dataset published by van de Vijver et al., (van de Vijver et al., 2002) downloaded from http://www.rii.com/publications/2002/default.html except that matching was done directly using gene names.

Association of copy number with survival. Stage 4 samples were excluded from all the outcome-related analyses; and disease-specific survival and time to distant recurrence were used as the two endpoints. We identified clinical variables independently associated with outcome endpoints by first using univariate Cox-proportional hazards model to identify clinical variables individually associated with the outcomes and then identifying the subset of variables significant in the additive multivariate model which included all significant variables from univariate analyses. Significance was declared at the 0.05 level. As demonstrated in (Willenbrock and Fridlyand, 2005), analyzing segmented data greatly increases power to detect true significant associations without increasing the false positive rate. Therefore, we used smoothed imputed data with outliers as described above to identify significant associations of low-level copy number changes with outcome endpoints. P-values were adjusted using False Discovery Rate (FDR) and a genome association was considered significant if its FDR was less than 0.05. A Cox proportional model also was used to associate the total number of copy number transitions and amount of genome gained and lost with survival; overall and within expression subtypes. P-values were not adjusted for FDR for these two analyses due to their targeted nature and significance was declared at the 0.05 level.

Regions of high level amplification were declared recurrent when present in at least 5 samples. The BAC array probes were further manually grouped to form groups of contiguous regions thereby referred to as amplicons, and singletons were excluded. Each sample was further classified as amplified for a given amplicon if it contained at least one amplified probe in the amplicon region. We tested all amplicons for association with the outcome variables by fitting univariate and multivariate Cox-proportional models with and without clinical variables and assessing significance of the standardized Cox-proportional coefficient. Significance was declared at unadjusted p-value<0.05.

Association of copy number with expression. The presence of an overall dosage effect was assessed by subdividing each chromosomal arm into non-overlapping 20 Mb bins and computing the average of cross-Pearson-correlations for all gene transcript-BAC probe pairs that mapped to that bin. We also calculated Pearson correlations and corresponding p-values between expression level and copy number for each gene transcript. Each transcript was assigned an observed copy number of the nearest mapped BAC array probe. 80% of gene transcripts had a nearest clone within 1 Mbp and 50% had a clone within 400 kbp. Correlation between expression and copy number was only computed for the gene transcripts whose absolute assigned copy number exceeded 0.2 in at least 5 samples. This was done to avoid spurious correlations in the absence of real copy number changes. We used conservative Holm p-value adjustment to correct for multiple testing. Gene transcripts with an adjusted p-value<0.05 were considered to have expression levels that were highly significantly affected by gene dosage. This corresponded to a minimum Pearson correlation of 0.44.

Associations of transcription and CNA in regions of amplification with outcome in tumors without particular amplicons. We assessed the associations of levels of transcripts in regions of amplifications with survival or distant recurrence in tumors without amplifications in order to find genes that might contribute to progression when deregulated by mechanisms other than amplification (e.g. we assessed associations between expression levels of the genes mapping to the 8p11-12 amplicon and survival in samples without 8p11-12 amplification. We performed separate cox-proportional regressions for disease-specific survival and distant recurrence. Stage 4 samples were excluded from all analyses. Testing for functional enrichment. We used the gene ontology statistics tool, GoStat (Beissbarth and Speed, 2004) to test whether gene transcripts with strongest dosage effects were enriched for particular functional groups. The p-values were adjusted using False Discovery Rate. The categories were considered significantly overrepresented if the FDR-adjusted p-value was less than 0.001. Since expressed genes were significantly more likely to show dosage effects than non expressed genes (p-value<2.2e-16, Wilcoxon rank sum test), GoStat comparisons were performed only for expressed genes. Specifically, GO categories for 1734 expressed probes with significant dosage effect (Holm p-value<0.05) were compared with those for 3026 expressed probes with no dosage effect (Pearson correlation<0.1).

### Example 5

### Probe Preparation

Methods of preparing probes are well known to those of skill in the art (see, e.g. Sambrook et al., Molecular Cloning: A Laboratory Manual (2nd ed.), Vols. 1-3, Cold Spring Harbor Laboratory, (1989) or Current Protocols in Molecular Biology, F. Ausubel et al., ed. Greene Publishing and Wiley-Interscience, New York (1987)), which are hereby incorporated by reference.

Prior to use, constructs are fragmented to provide smaller nucleic acid fragments that easily penetrate the cell and hybridize to the target nucleic acid. Fragmentation can be by any of a number of methods well known to hose of skill in the art. Preferred methods include treatment with a restriction enzyme to selectively cleave the molecules, or alternatively to briefly heat the nucleic acids in the presence of Mg²⁺. Probes are preferably fragmented to an average fragment length ranging from about 50 bp to about 2000 bp, more preferably from about 100 bp to about 1000 bp and most preferably from about 150 bp to about 500 bp.

Methods of labeling nucleic acids are well known to those of skill in the art. Preferred labels are those that are suitable for use with in situ hybridization. The nucleic acid probes may be detectably labeled prior to the hybridization reaction. Alternatively, a detectable label which binds to the hybridization product may be used. Such detectable labels include any material having a detectable physical or chemical property and have been well-developed in the field of immunoassays.

As used herein, a “label” is any composition detectable by spectroscopic, photochemical, biochemical, immunochemical, or chemical means. Useful labels in the present invention include radioactive labels (e.g, ³²P, ¹²⁵I, ¹⁴C, ³H, and ³⁵S), fluorescent dyes (e.g. fluorescein, rhodamine, Texas Red, etc.), electron-dense reagents (e.g. gold), enzymes (as commonly used in an ELISA), calorimetric labels (e.g. colloidal gold), magnetic labels (e.g. DYNABEADS™), and the like. Examples of labels which are not directly detected but are detected through the use of directly detectable label include biotin and dioxigenin as well as haptens and proteins for which labeled antisera or monoclonal antibodies are available.

The particular label used is not critical to the present invention, so long as it does not interfere with the in situ hybridization of the stain. However, stains directly labeled with fluorescent labels (e.g. fluorescein-12-dUTP, Texas Red-5-dUTP, etc.) are preferred for chromosome hybridization.

A direct labeled probe, as used herein, is a probe to which a detectable label is attached. Because the direct label is already attached to the probe, no subsequent steps are required to associate the probe with the detectable label. In contrast, an indirect labeled probe is one which bears a moiety to which a detectable label is subsequently bound, typically after the probe is hybridized with the target nucleic acid.

In addition the label must be detectable in as low copy number as possible thereby maximizing the sensitivity of the assay and yet be detectible above any background signal. Finally, a label must be chosen that provides a highly localized signal thereby providing a high degree of spatial resolution when physically mapping the stain against the chromosome. Particularly preferred fluorescent labels include fluorescein-12-dUTP and Texas Red-5-dUTP.

The labels may be coupled to the probes in a variety of means known to those of skill in the art. In a preferred embodiment the nucleic acid probes will be labeled using nick translation or random primer extension (Rigby, et al. J. Mol. Biol., 113: 237 (1977) or Sambrook, et al., Molecular Cloning—A Laboratory Manual, Cold Spring Harbor Laboratory, Cold Spring Harbor, N.Y. (1985)).

One of skill in the art will appreciate that the probes of this invention need not be absolutely specific for the targeted 8p11-12, 11q13-14, 17q11-12 or 20q13 regions of the genome. Rather, the probes are intended to produce “staining contrast”. “Contrast” is quantified by the ratio of the probe intensity of the target region of the genome to that of the other portions of the genome. For example, a DNA library produced by cloning a particular chromosome (e.g. chromosome 7) can be used as a stain capable of staining the entire chromosome. The library contains both sequences found only on that chromosome, and sequences shared with other chromosomes. Roughly half the chromosomal DNA falls into each class. If hybridization of the whole library were capable of saturating all of the binding sites on the target chromosome, the target chromosome would be twice as bright (contrast ratio of 2) as the other chromosomes since it would contain signal from the both the specific and the shared sequences in the stain, whereas the other chromosomes would only be stained by the shared sequences. Thus, only a modest decrease in hybridization of the shared sequences in the stain would substantially enhance the contrast. Thus, contaminating sequences which only hybridize to non-targeted sequences, for example, impurities in a library can be tolerated in the stain to the extent that the sequences do not reduce the staining contrast below useful levels.

### Example 6

### In Situ Hybridization

Generally, in situ hybridization comprises the following major steps: (1) fixation of tissue or biological structure to analyzed; (2) prehybridization treatment of the biological structure to increase accessibility of target DNA, and to reduce nonspecific binding; (3) hybridization of the mixture of nucleic acids to the nucleic acid in the biological structure or tissue; (4) posthybridization washes to remove nucleic acid fragments not bound in the hybridization and (5) detection of the hybridized nucleic acid fragments. The reagents used in each of these steps and their conditions for use vary depending on the particular application.

In some applications it is necessary to block the hybridization capacity of repetitive sequences. In this case, human genomic DNA is used as an agent to block such hybridization. The preferred size range is from about 200 bp to about 1000 bases, more preferably between about 400 to about 800 bp for double stranded, nick translated nucleic acids.

Hybridization protocols for the particular applications disclosed here are described in Pinkel et al. Proc. Natl. Acad. Sci. USA, 85: 9138-9142 (1988) and in EPO Pub. No. 430,402. Suitable hybridization protocols can also be found in Methods in Molecular Biology Vol. 33, In Situ Hybridization Protocols, K. H. A. Choo, ed., Humana Press, Totowa, N.J., (1994). +In a particularly preferred embodiment, the hybridization protocol of Kallioniemi et al., ERBB2 amplification in breast cancer analyzed by fluorescence in situ hybridization. Proc Natl Acad Sci USA, 89: 5321-5325 (1992) is used.

Typically, it is desirable to use dual color FISH, in which two probes are utilized, each labeled by a different fluorescent dye. A test probe that hybridizes to the region of interest is labeled with one dye, and a control probe that hybridizes to a different region is labeled with a second dye. A nucleic acid that hybridizes to a stable portion of the chromosome of interest, such as the centromere region, is often most useful as the control probe. In this way, differences between efficiency of hybridization from sample to sample can be accounted for.

The FISH methods for detecting chromosomal abnormalities can be performed on nanogram quantities of the subject nucleic acids. Paraffin embedded tumor sections can be used, as can fresh or frozen material. Because FISH can be applied to the limited material, touch preparations prepared from uncultured primary tumors can also be used (see, e.g., Kallioniemi, A. et al., Cytogenet. Cell Genet. 60: 190-193 (1992)). For instance, small biopsy tissue samples from tumors can be used for touch preparations (see, e.g., Kallioniemi, A. et al., Cytogenet. Cell Genet. 60: 190-193 (1992)). Small numbers of cells obtained from aspiration biopsy or cells in bodily fluids (e.g., blood, urine, sputum and the like) can also be analyzed. For prenatal diagnosis, appropriate samples will include amniotic fluid and the like.

### Example 7

### Quantitative PCR

Elevated gene expression is detected using quantitative PCR. Primers can be created to detect sequence amplification by signal amplification in gel electrophoresis. As is known in the art, primers or oligonucleotides are generally 15-40 bp in length, and usually flank unique sequence that can be amplified by methods such as polymerase chain reaction (PCR) or reverse transcriptase PCR (RT-PCR, also known as real-time PCR). Methods for RT-PCR and its optimization are known in the art. An example is the PROMEGA PCR Protocols and Guides, found at URL:<http://www.promega.com/guides/pcr guide/default.htm>, and hereby incorporated by reference. Currently at least four different chemistries, TaqMan® (Applied Biosystems, Foster City, Calif., USA), Molecular Beacons, Scorpions® and SYBR® Green (Molecular Probes), are available for real-time PCR. All of these chemistries allow detection of PCR products via the generation of a fluorescent signal. TaqMan probes, Molecular Beacons and Scorpions depend on Förster Resonance Energy Transfer (FRET) to generate the fluorescence signal via the coupling of a fluorogenic dye molecule and a quencher moiety to the same or different oligonucleotide substrates. SYBR Green is a fluorogenic dye that exhibits little fluorescence when in solution, but emits a strong fluorescent signal upon binding to double-stranded DNA.

Two strategies are commonly employed to quantify the results obtained by real-time RT-PCR; the standard curve method and the comparative threshold method. In this method, a standard curve is first constructed from an RNA of known concentration. This curve is then used as a reference standard for extrapolating quantitative information for mRNA targets of unknown concentrations. Another quantitation approach is termed the comparative Cₜ method. This involves comparing the Cₜ values of the samples of interest with a control or calibrator such as a non-treated sample or RNA from normal tissue. The Cₜ values of both the calibrator and the samples of interest are normalized to an appropriate endogenous housekeeping gene.

### Example 8

### High Throughput Screening

High throughput screening (HTS) methods are used to identify compounds that inhibit candidate genes which are related to drug resistance and reduced survival rate. HTS methods involve providing a combinatorial chemical or peptide library containing a large number of potential therapeutic compounds. Such “libraries” are then screened in one or more assays, as described herein, to identify those library members (particular peptides, chemical species or subclasses) that display the desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al, Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. Nos. 5,506,337; benzodiazepines, 5,288,514, and the like).

Devices for the preparation of combinatorial libraries are commercially available (see, e.g., ECIS™, Applied BioPhysics Inc., Troy, N.Y., MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### Example 9

### Inhibitor Oligonucleotide and RNA Interference (RNAi) Sequence Design

Known methods are used to identify sequences that inhibit candidate genes which are related to drug resistance and reduced survival rate. Such inhibitors may include but are not limited to, siRNA oligonucleotides, antisense oligonucleotides, peptide inhibitors and aptamer sequences that bind and act to inhibit PVT1 expression and/or function.

RNA interference is used to generate small double-stranded RNA (small interference RNA or siRNA) inhibitors to affect the expression of a candidate gene generally through cleaving and destroying its cognate RNA. Small interference RNA (siRNA) is typically 19-22 nt double-stranded RNA. siRNA can be obtained by chemical synthesis or by DNA-vector based RNAi technology. Using DNA vector based siRNA technology, a small DNA insert (about 70 bp) encoding a short hairpin RNA targeting the gene of interest is cloned into a commercially available vector. The insert-containing vector can be transfected into the cell, and expressing the short hairpin RNA. The hairpin RNA is rapidly processed by the cellular machinery into 19-22 nt double stranded RNA (siRNA). In a preferred embodiment, the siRNA is inserted into a suitable RNAi vector because siRNA made synthetically tends to be less stable and not as effective in transfection.

siRNA can be made using methods and algorithms such as those described by Wang L, Mu FY. (2004) A Web-based Design Center for Vector-based siRNA and siRNA cassette. Bioinformatics. (In press); Khvorova A, Reynolds A, Jayasena S D. (2003) Functional siRNAs and miRNAs exhibit strand bias. Cell. 115(2):209-16; Harborth J, Elbashir S M, Vandenburgh K, Manning a H, Scaringe S A, Weber K, Tuschl T. (2003) Sequence, chemical, and structural variation of small interfering RNAs and short hairpin RNAs and the effect on mammalian gene silencing. Antisense Nucleic Acid Drug Dev. 13(2):83-105; Reynolds A, Leake D, Boese Q, Scaringe S, Marshall WS, Khvorova A. (2004) Rational siRNA design for RNA interference. Nat Biotechnol. 22(3):326-30 and Ui-Tei K, Naito Y, Takahashi F, Haraguchi T, Ohki-Hamazaki H, Juni A, Ueda R, Saigo K. (2004) Guidelines for the selection of highly effective siRNA sequences for mammalian and chick RNA interference. Nucleic Acids Res. 32(3):936-48, which are hereby incorporated by reference.

Other tools for constructing siRNA sequences are web tools such as the siRNA Target Finder and Construct Builder available from GenScript (http://www.genscript.com), Oligo Design and Analysis Tools from Integrated DNA Technologies (URL:<http://www.idtdna.com/SciTools/SciTools.aspx>), or siDESIGN™ Center from Dharmacon, Inc. (URL:<http://design.dharmacon.com/default.aspx?source=0>). siRNA are suggested to built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, since the function of siRNAs is to cleave mRNA sequences, it is important to use the mRNA nucleotide sequence and not the genomic sequence for siRNA design, although as noted in the Examples, the genomic sequence can be successfully used for siRNA design. However, designs using genomic information might inadvertently target introns and as a result the siRNA would not be functional for silencing the corresponding mRNA.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off-target effects is to screen the siRNA for target specificity.

The siRNA can be modified on the 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

Antisense oligonucleotides (“oligos”) can be designed to inhibit candidate gene function. Antisense oligonucleotides are short single-stranded nucleic acids, which function by selectively hybridizing to their target mRNA, thereby blocking translation. Translation is inhibited by either RNase H nuclease activity at the DNA:RNA duplex, or by inhibiting ribosome progression, thereby inhibiting protein synthesis. This results in discontinued synthesis and subsequent loss of function of the protein for which the target mRNA encodes.

In a preferred embodiment, antisense oligos are phosphorothioated upon synthesis and purification, and are usually 18-22 bases in length. It is contemplated that the candidate gene antisense oligos may have other modifications such as 2′-O-Methyl RNA, methylphosphonates, chimeric oligos, modified bases and many others modifications, including fluorescent oligos.

In a preferred embodiment, active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5 μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20° C. and stable for several weeks.

Aptamer sequences which bind to specific RNA or DNA sequences can be made. Aptamer sequences can be isolated through methods such as those disclosed in co-pending U.S. patent application Ser. No. 10/934,856, entitled, “Aptamers and Methods for their Invitro Selection and Uses Thereof,” which is hereby incorporated by reference.

It is contemplated that the sequences described herein may be varied to result in substantially homologous sequences which retain the same function as the original. As used herein, a polynucleotide or fragment thereof is “substantially homologous” (or “substantially similar”) to another if, when optimally aligned (with appropriate nucleotide insertions or deletions) with the other polynucleotide (or its complementary strand), using an alignment program such as BLASTN (Altschul, S. F., Gish, W., Miller, W., Myers, E.W. & Lipman, D. J. (1990) “Basic local alignment search tool.” J. Mol. Biol. 215:403-410), and there is nucleotide sequence identity in at least about 80%, preferably at least about 90%, and more preferably at least about 95-98% of the nucleotide bases.

### Example 10

### Inhibition of ADAM9 Induces Cell Apoptosis

It was found that silencing of ADAM9 inhibits breast cancer cell growth and cell proliferation and inhibition of ADAM9 expression in breast cancer cells induces cell apoptosis. Thus, ADAM9 is implicated in proliferative aspects of breast cancer pathophysiology and serves as a possible therapeutic target in breast cancer.

A comprehensive study of gene expression and copy number in primary breast cancers and breast cancer cell lines was carried out, whereby we identified a region of high level amplification on chromosome 8p11 that is associated with reduced survival duration. The metalloproteinase-like, disintegrin-link and cysteine-rich protein, ADAM9, identified herein, maps to the region of amplification at 8p11. siRNA knockdown was applied to explore how amplification and over-expression of this particular gene play a role in breast cancer pathophysiology and to determine if this gene may be a valuable therapeutic target.

We transiently transfected 83 nM of siRNA for ADAM9 into T47D, BT549, SUM52PE, 600 MPE and MCF10A breast cancer cell lines. Non-specific siRNA served as a negative control. Cell viability/proliferation was evaluated by CellTiter-Glo® luminescent cell viability assay (CTG, Promega), cell apoptosis was assayed using YoPro-1 and Hoechst staining and cell cycle inhibition was assessed by measuring BrdU incorporation. All cellular measurements were made in adhered cells using the Cellomics high content scanning instrument. All assays were run at 3, 4, 5 and 6 days post transfection.

Briefly, the siRNA transfection protocol was as follows. Cells are plated and grown to 50-70% confluency and transfected using DharmaFECT1. In tubes, mix: Tube A: total volume 10 ul 9.5 uL SFM media+0.5siRNA (varied according to the experiment design); Tube B: total volume 10 ul 9.8 uL SFM media+0.2 DharmaFECT1. Incubate tubes for 5 min. During this incubation, remove media from target cells and replace with SFM in each well. Add contents of Tube B to Tube A and mix gently. Incubate for 20 min at room temperature. Add 20 uL mixture solution dropwise to each well (final volume=100 uL). Leave for 4 h, aspirate off media and replace with full growth media and allow cells to grow for several days.

Cell growth analysis was carried out using the CellTitter-Glo® Luminescent Cell Ciability Assay (Promega Cat#G7571/2/3). The luminescence signal of viable cells as measured the amount of ATP detected in the plates were read using a custom plate reader and program.

BrdU Staining and Fixation for Cellomics were used to measure cell proliferation and cell cycle analysis. To incorporate BrdU and fix the cells 10 uM final concentration of BrdU (Sigma #B5002) was added directly to cell media and pulsed for 30 minutes in tissue culture incubator. The media was removed and the cells washed 2× with 1×PBS and then 70% EtOH added to cover cells and fix for overnight at 4° C. Next day the 70% EtOH was removed and cells allowed to dry. Then 2N HCl was added and cells incubated at room temperature for 5-10 minutes, then removed and 1×PBS added to neutralize. Diluted anti-BrdU antibody (Mouse anti-BrdU Clone 3D4 (BD Pharmingen #555627)) 1:100 in 1×PBS/0.5% Tween-20. Anti-BrdU was added to cells (50 ul-96 well plate; 200 ul-24 well plate) and incubated for 45-60 minutes at room temperature on a rocker. Antibody was aspirated and cells washed 2× with 1×PBS/0.5% Tween-20. Rabbit Anti-mouse Alexa Fluor 488 (Invitrogen #A-11059) was diluted 1:250 in 1×PBS/0.5% Tween-20. Secondary antibody was added to cells and incubated 30-60 minutes at room temperature on a rocker then washed 3× with 1×PBS/0.5% Tween-20. After the last wash was removed and cells were incubated with 1 ug/ml Hoechst 33342 (Sigma #B2261) diluted in 1×PBS for 45 minutes at room temperature on a rocker. Cells were washed and covered with 1×PBS. Plates were scanned or stored at 4° C. for later scanning on Cellomics.

YoPro-1 Staining for Cellomics was used for cell apoptosis analysis. Add YoPro-1(Final use at 1 ug/ml) and Hoechst (Final use at 10 ug/ml) directly to cell media. Place in 37° C. incubator for 30 min. Then read directly on Cellomics Significant knockdown of ADAM9 was achieved in BT549 and T47D cells transfected with siRNA-ADAM9 for 48 hr, 72 hr and 96 hr. Silencing of ADAM9 significantly reduced the proliferation of breast cancer cells and inhibited the BrdU incorporation after treatment with siRNA compared to controls. Knockdown of ADAM9 in breast cancer cells also induced significant levels of apoptosis. Furthermore, we found that cells had very good response when the concentration of siRNA-ADAM9 were higher than 30 nM. The current results suggested that silencing expression of ADAM9 is a novel approach for inhibition of breast cancer cell growth. ADAM9 may serve as a new candidate therapeutic target for treatment of breast cancer with poor outcome.

### Example 11

### Inhibition of Genes Encoded by the 11q13 Amplicon

As described above, the 11q13 amplicon encodes ten genes or non-coding RNA transcripts that appear likely to contribute to the pathophysiology of breast cancer and that are potential therapeutic targets. None of these genes are considered druggable based on predicted protein folding characteristics. However, all are candidates for siRNA therapeutic attack. We applied an efficient siRNA transfection strategy as explained in Example 9 to assess the therapeutic potential of siRNAs against genes encoded in the region of recurrent amplification at 11q13.

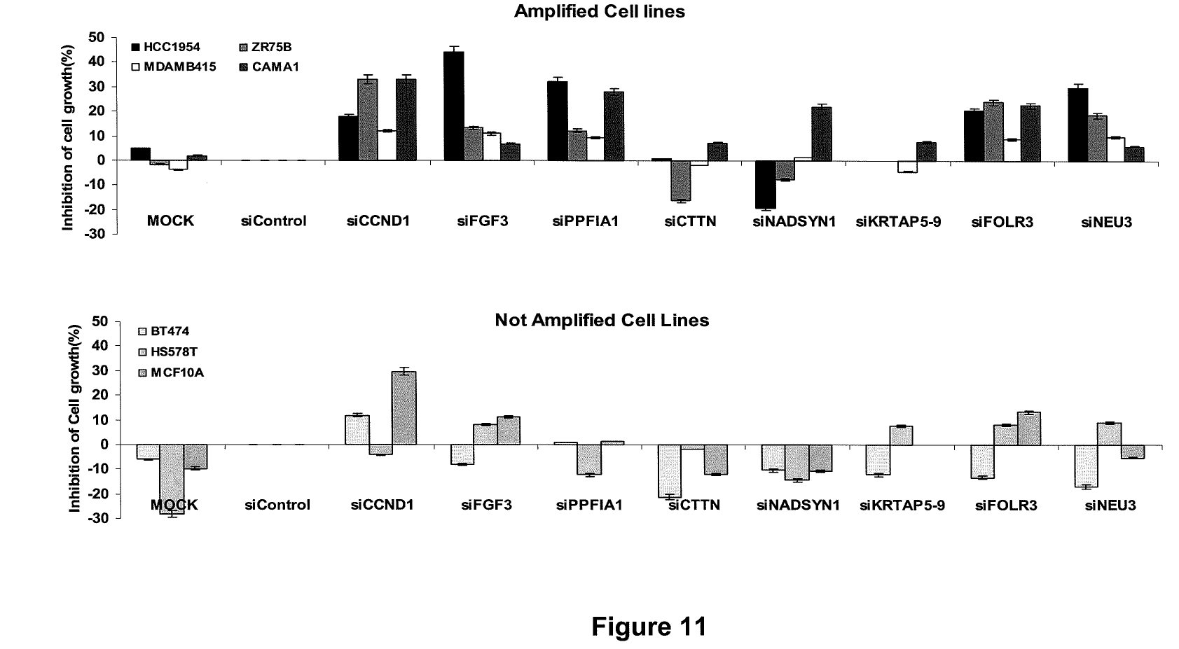

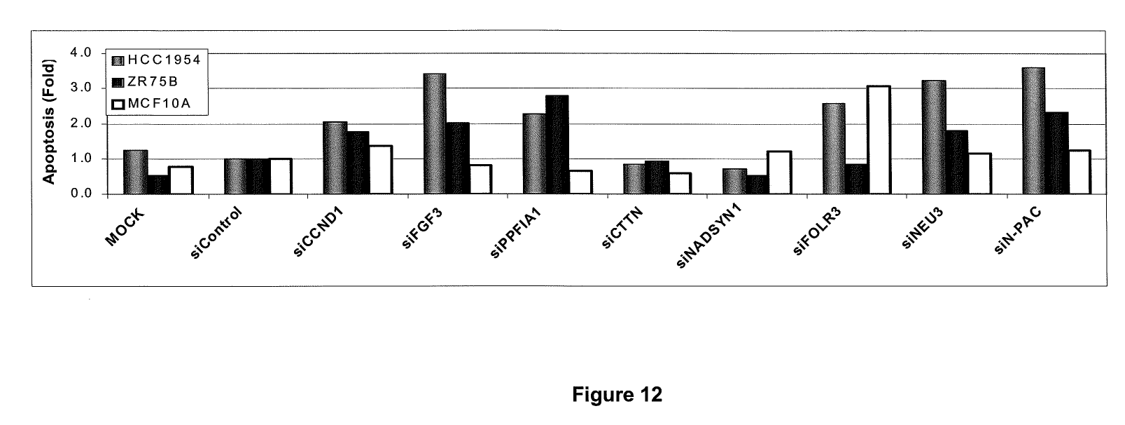

We transiently transfect 50 nM of siRNAs targeting these genes (4 individual siRNAs per gene, Table 8) in cell lines amplified at 11q13 (HCC1954, ZR75B, MDAMB415 and CAMA1) and not amplified (BT474, HS578T and MCF10A). Non-specific siRNA served as a negative control Viable cell number and apoptosis index were measured for each siRNA. These analyses showed that silencing of CCND1, FGF3, PPFIA1, FOLR3, and NEU3 reduced the cell growth of 11q13-amplified breast cancer cells compared to unamplified controls (FIG. 11). Knockdown of FGF3, PPFIA1 and NEU3 also induced cell apoptosis in amplified cell lines (HCC1954 and ZR75B), but not in non-amplified lines MCF10A (FIG. 12).

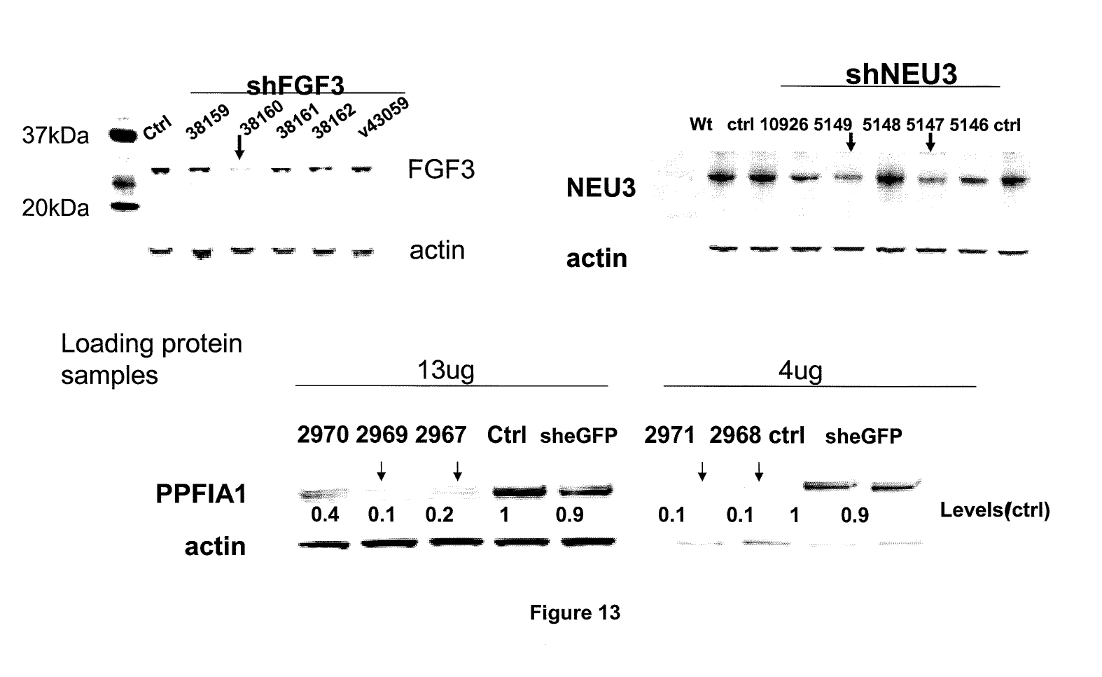

To further validated the therapeutic potential of targeting FGF3, PPFIA1 and NEU3, we packaged shRNA lenti-virus (5 shRNAs for each gene, Open Biosystems Inc. Table 9 using the third generation lenti-virus packaging system and infected breast cells in which amplified/overexpressed FGF3, PPFIA1 and NEU3 are overexpressed with these lentiviral shRNAs. Knockdown efficiency was then measured by western blot. We identified successful clones marked with arrows (at least one clone for each gene) that can knock down more than 80% protein of the target genes (FIG. 13).

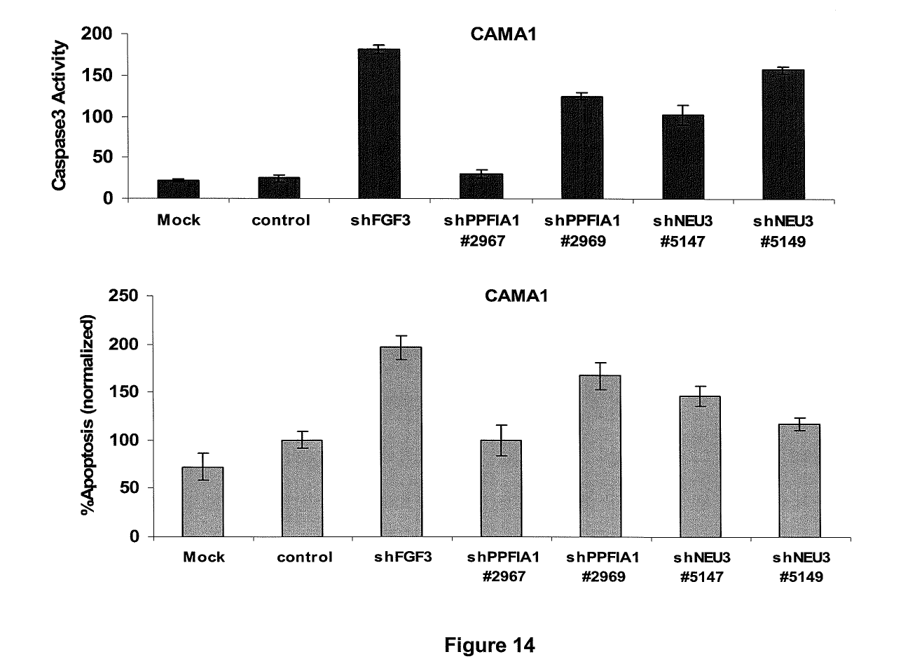

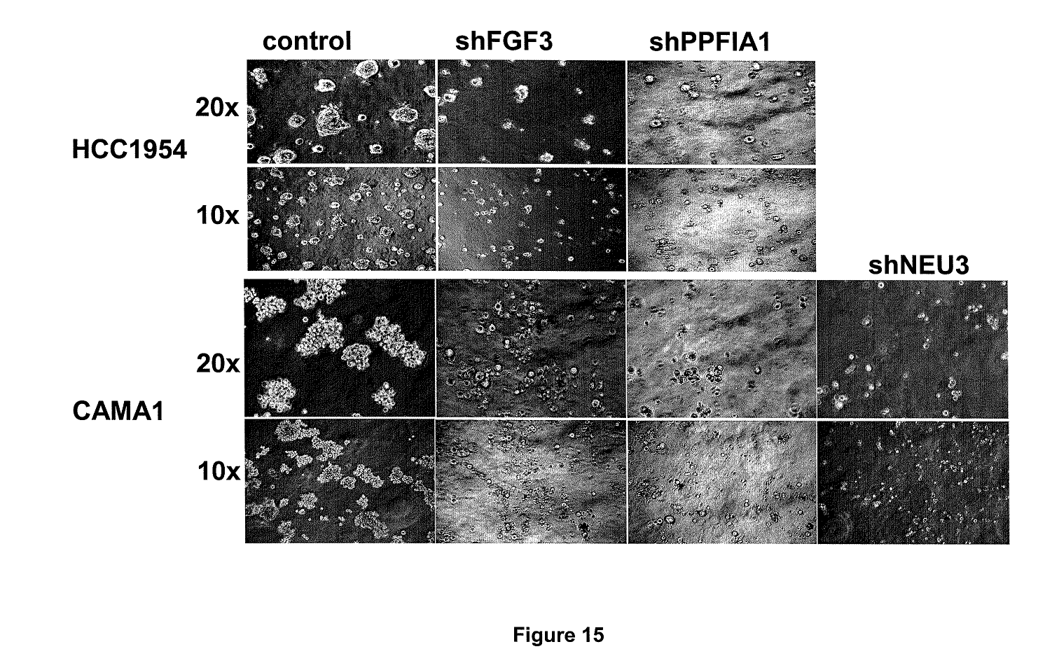

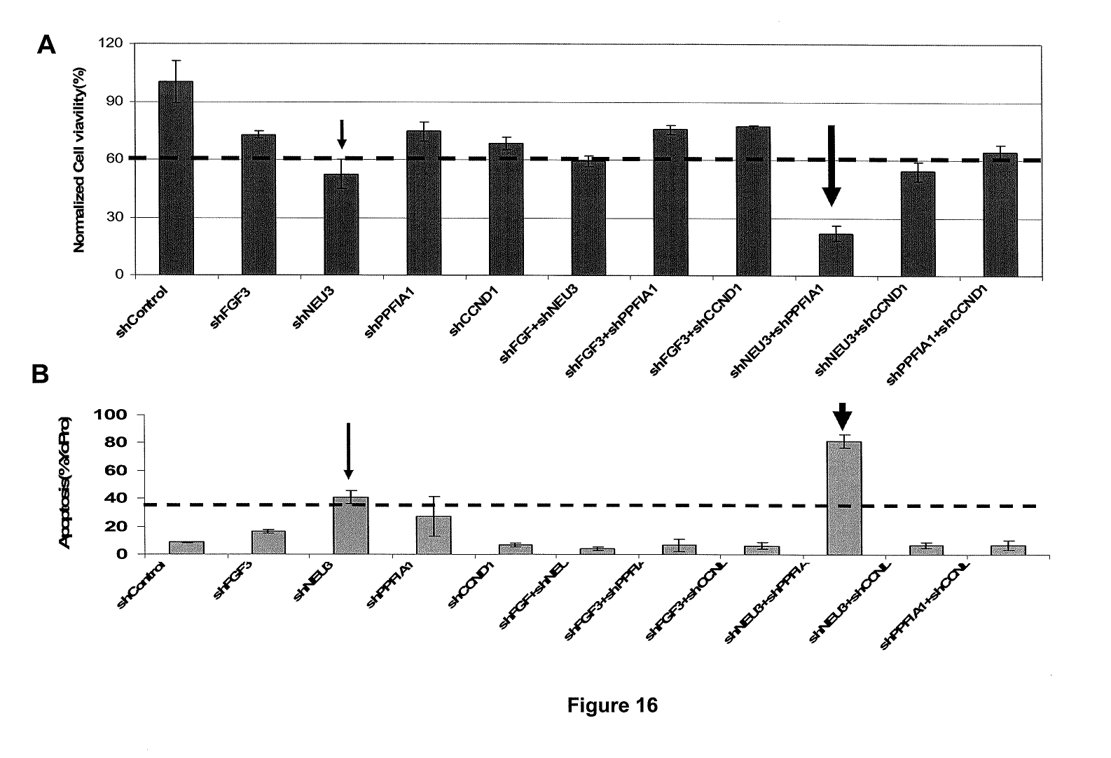

Knockdown of FGF3, PPFIA1, and NEU3 also induced cell apoptosis and inhibited cell growth in 3D culture. We measured cell apoptosis by caspase3 activity and/or YoPRO plus Hoechst staining after cells infected with shRNAs using methodology described in Example 9. We found that knockdown of FGF3, PPFIA1 and NEU3 by shRNA significantly increased cell apoptosis in breast cancer cells (FIG. 14). We also examined the effects in a 3D culture system, which can mimic in vivo environments. Our data showed that when each of these three genes was silenced in CAMA1 and HCC1954 cells, the colonies were much smaller in comparison to the shRNA control that was also cultured in the 3D culture system (FIG. 15).

Combinational knockdown of genes at 11q13 amplicon has the synergistic effect in breast cancer cells.

To evaluate the synergistic effect on knockdown of candidate therapeutic targets FGF3, PPFIA1, NEU3 and CCND1, we infected breast cancer cells with shRNAs lentivirus individually and/or combinationally. Our data showed that combinational knockdown of NEU3 and PPFIA1 significantly inhibited cell growth (FIG. 16A). Combinational knockdown of NEU3 and PPFIA1 also increased cell apoptosis dramatically (FIG. 16B), which is consistent with our cell growth data. Our findings indicated that knockdown of NEU3 and PPFIA1 at the same time has a singificant synergistic effect on cell growth inhibition and cell apoptosis. In summary, the data in these examples show that FGF3, PPFIA1, and NEU3 and the combination of NEU3 and PPFIA1, in particular, are potential therapeutic targets in breast cancer cells.

### Example 12

### Inhibition of Genes Encoded by the 20q13 Amplicon

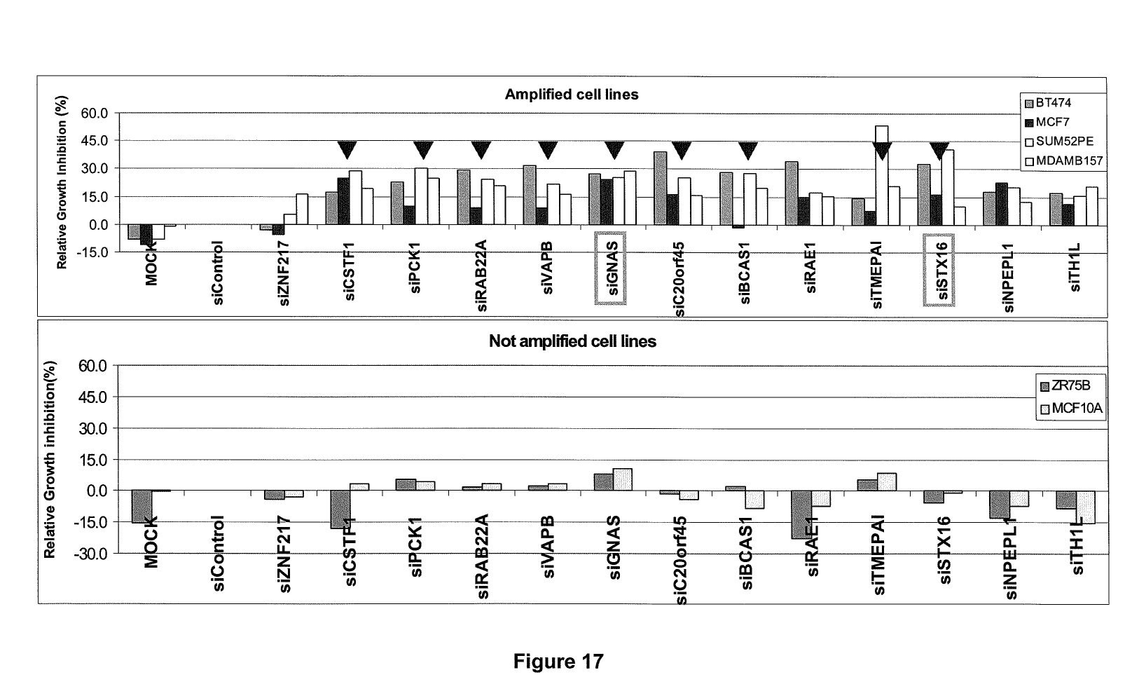

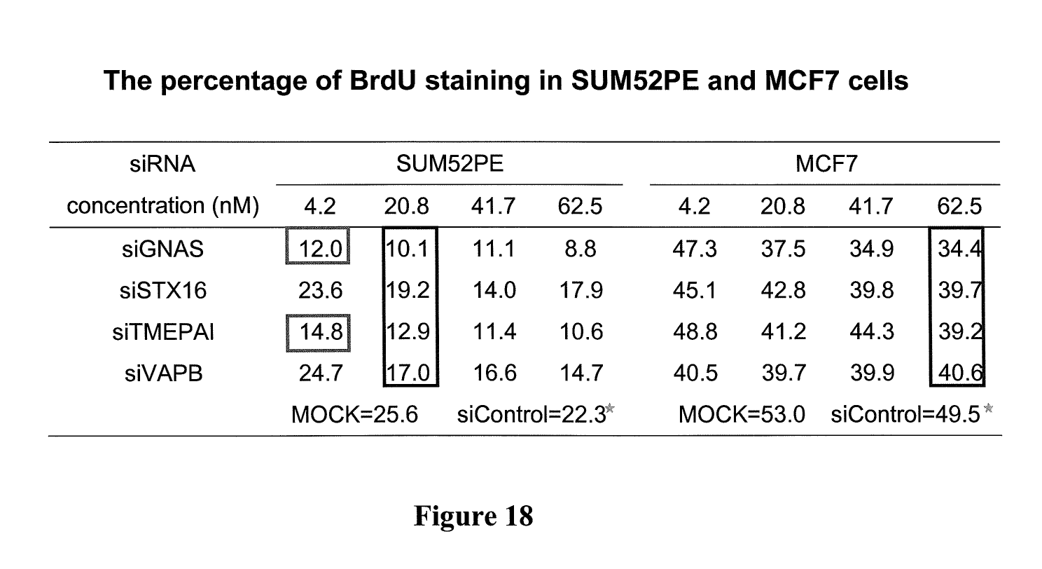

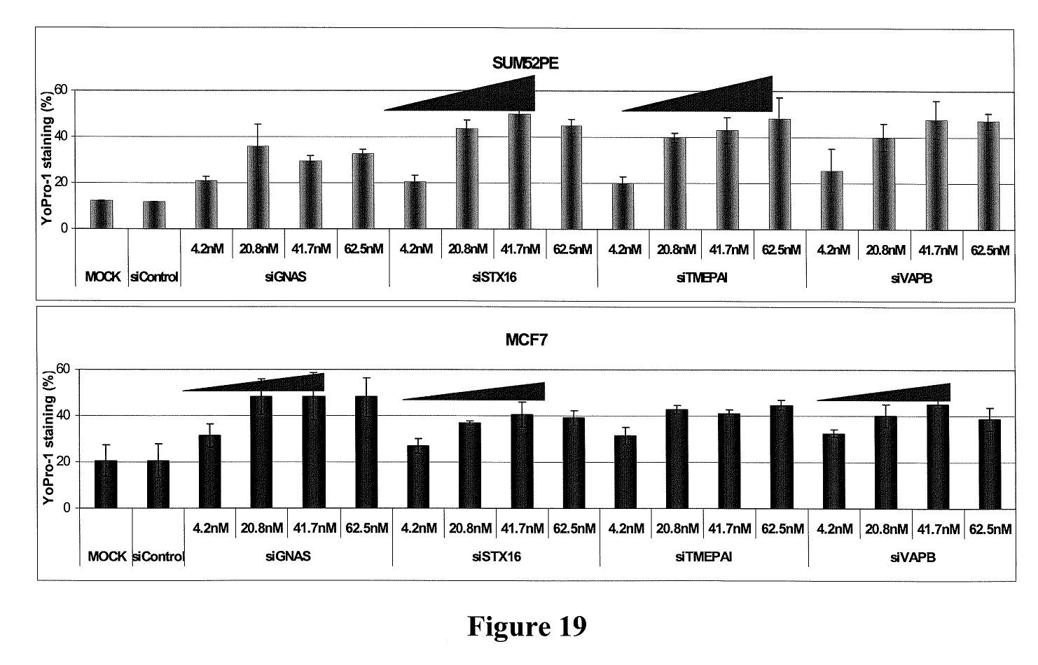

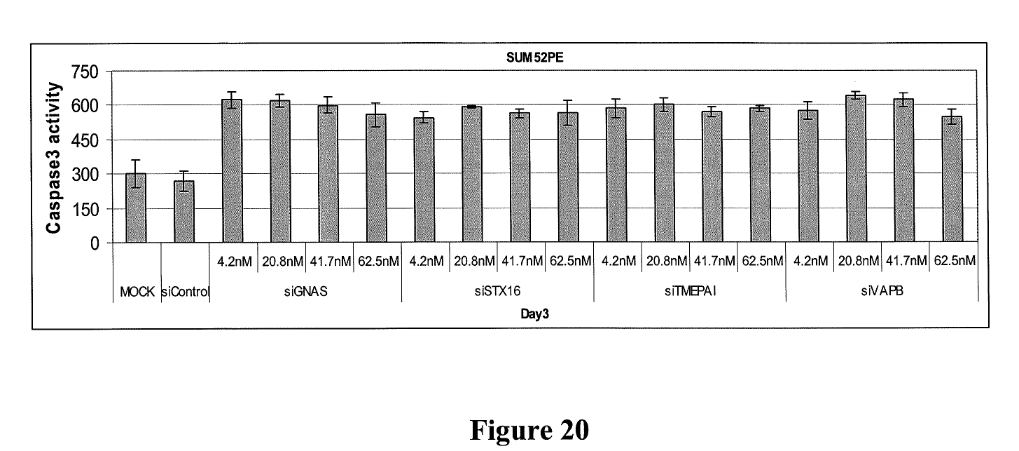

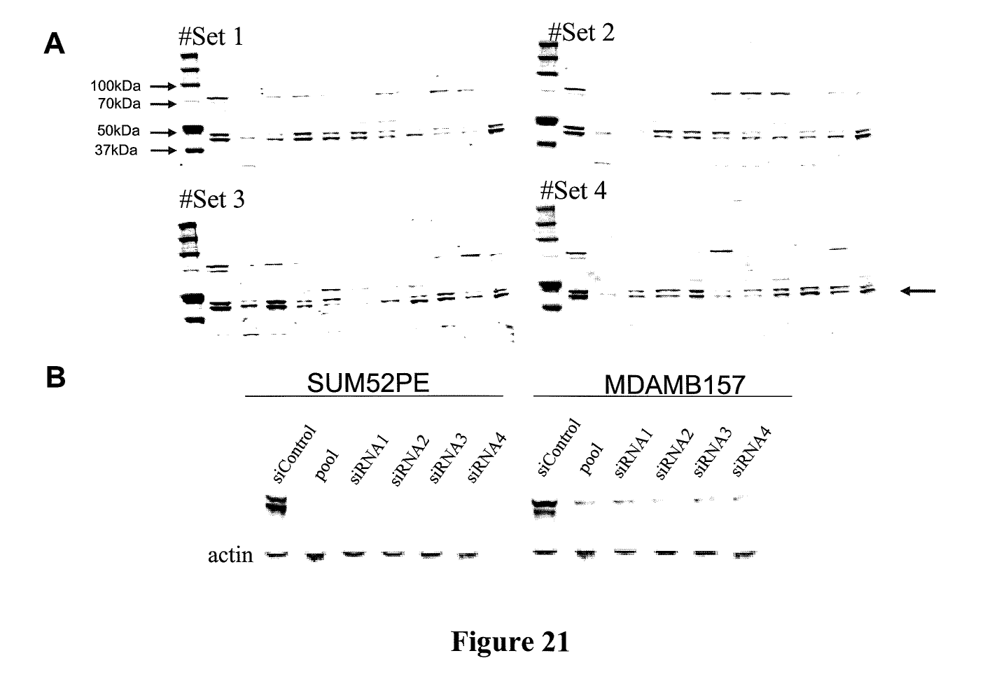

As described above, the 20q13 amplicon encodes fourteen genes or non-coding RNA transcripts that appear likely to contribute to the pathophysiology of breast cancer and that are potential therapeutic targets. None of these genes are considered druggable based on predicted protein folding characteristics. However, all are candidates for siRNA therapeutic attack. We applied an efficient siRNA transfection strategy as explained in Example 9 to assess the therapeutic potential of siRNAs against genes encoded in the region of recurrent amplification at 20q13. We transiently transfected 50 nM of siRNAs (Table 10) targeting these genes (4 individual siRNAs per gene) in cell lines amplified at 20q13 (BT474, MCF7, MDAMB157 and SUM52PE) and not amplified (MCF10A and ZR75B). Non-specific siRNA served as a negative control. Viable cell number, proliferation and apoptosis index were measured for each siRNA using the assays described in Example 9. These analyses showed that silencing of CSTF1, PCK1, RAB22A, VAPB, GNAS, C20orf45, BCAS1, TMEPA1 and STX16 reduced the cell growth of 20q13-amplified breast cancer cells compared to unamplified controls (FIG. 17). Knockdown of VAPB, GNAS, TMEPAI and STX16 also inhibited cell proliferation (the percentage of S-phase cells) and induced cell apoptosis in amplified cell lines MCF7 and SUM52PE cells (FIGS. 18 and 19). Caspase 3 activity also increased in SUM52PE cells treated for 72 hours with VAPB, GNAS, TMEPAI and STX16 siRNAs (FIG. 20). siGNAS also knocked down Gs transcripts in the amplified cell lines (FIG. 21). These results indicate that therapeutic strategies, e.g., targeting CSTF1, PCK1, VAPB, GNAS, BCAS1, TMEPAI and STX16 genes, may be particularly effective in treating patients with 20q13 amplification.

### CITATIONS

Akagi, K., Suzuki, T., Stephens, R. M., Jenkins, N. A., and Copeland, N. G. (2004). RTCGD: retroviral tagged cancer gene database. Nucleic Acids Res 32, D523-527.
Al-Kuraya, K., Schraml, P., Torhorst, J., Tapia, C., Zaharieva, B., Novotny, H., Spichtin, H., Maurer, R., Mirlacher, M., Kochli, O., et al. (2004). Prognostic relevance of gene amplifications and coamplifications in breast cancer. Cancer Res 64, 8534-8540.
Albertson, D. G., Collins, C., McCormick, F., and Gray, J. W. (2003). Chromosome aberrations in solid tumors. Nat Genet 34, 369-376.
Babu, J. R., Jeganathan, K. B., Baker, D. J., Wu, X., Kang-Decker, N., and van Deursen, J. M. (2003). Rael is an essential mitotic checkpoint regulator that cooperates with Bub3 to prevent chromosome missegregation. J Cell Biol 160, 341-353.
Barlund, M., Monni, O., Kononen, J., Cornelison, R., Torhorst, J., Sauter, G., Kallioniemi, O.-P., and Kallioniemi, A. (2000). Multiple genes at 17q23 undergo amplification and overexpression in breast cancer. Cancer Res 60, 5340-5344.
Barlund, M., Tirkkonen, M., Forozan, F., Tanner, M. M., Kallioniemi, O., and Kallioniemi, A. (1997). Increased copy number at 17q22-q24 by CGH in breast cancer is due to high-level amplification of two separate regions. Genes Chromosomes Cancer 20, 372-376.
Baylin, S. B., and Herman, J. G. (2000). DNA hypermethylation in tumorigenesis: epigenetics joins genetics. Trends Genet 16, 168-174.
Beissbarth, T., and Speed, T. P. (2004). GOstat: find statistically overrepresented Gene Ontologies within a group of genes. Bioinformatics 20, 1464-5 (2004). Bioinformatics 20, 1464-1465.
Blegen, H., Will, J. S., Ghadimi, B. M., Nash, H. P., Zetterberg, A., Auer, G., and Ried, T. (2003). DNA amplifications and aneuploidy, high proliferative activity and impaired cell cycle control characterize breast carcinomas with poor prognosis. Anal Cell Pathol 25, 103-114.
Braun, B. S., and Shannon, K. (2004). The sum is greater than the FGFR1 partner. Cancer Cell 5, 203-204.
Callagy, G., Pharoah, P., Chin, S. F., Sangan, T., Daigo, Y., Jackson, L., and Caldas, C. (2005). Identification and validation of prognostic markers in breast cancer with the complementary use of array-CGH and tissue microarrays. J Pathol 205, 388-396.
Cheng, K. W., Lahad, J. P., Kuo, W. L., Lapuk, A., Yamada, K., Auersperg, N., Liu, J., Smith-McCune, K., Lu, K. H., Fishman, D., et al. (2004). The RAB25 small GTPase determines aggressiveness of ovarian and breast cancers. Nat Med 10, 1251-1256.
Chin, K., de Solorzano, C. O., Knowles, D., Jones, A., Chou, W., Rodriguez, E. G., Kuo, W. L., Ljung, B. M., Chew, K., Myambo, K., et al. (2004). In situ analyses of genome instability in breast cancer. Nat Genet 36, 984-988.
Clairmont, C. A., Narayanan, L., Sun, K. W., Glazer, P. M., and Sweasy, J. B. (1999). The Tyr-265-to-Cys mutator mutant of DNA polymerase beta induces a mutator phenotype in mouse LN12 cells. Proc Natl Acad Sci USA 96, 9580-9585.
Deutschbauer, A. M., Jaramillo, D. F., Proctor, M., Kumm, J., Hillenmeyer, M. E., Davis, R. W., Nislow, C., and Giaever, G. (2005). Mechanisms of haploinsufficiency revealed by genome-wide profiling in yeast. Genetics 169, 1915-1925.
Esteva, F. J., Sahin, A. A., Cristofanilli, M., Coombes, K., Lee, S. J., Baker, J., Cronin, M., Walker, M., Watson, D., Shak, S., and Hortobagyi, G. N. (2005). Prognostic role of a multigene reverse transcriptase-PCR assay in patients with node-negative breast cancer not receiving adjuvant systemic therapy. Clin Cancer Res 11, 3315-3319.
Fraser, M. M., Watson, P. M., Fraig, M. M., Kelley, J. R., Nelson, P. S., Boylan, A. M., Cole, D. J., and Watson, D. K. (2005). CaSm-mediated cellular transformation is associated with altered gene expression and messenger RNA stability. Cancer Res 65, 6228-6236.
Fridlyand, J., Snijders, A. M., Ylstra, B., Li, H., Olshen, A., Segraves, R., Dairkee, S., Tokuyasu, T., Ljung, B. M., Jain, A. N., et al. (2006). Breast tumor copy number aberration phenotypes and genomic instability. BMC Cancer 6, 96.
Gelsi-Boyer, V., Orsetti, B., Cervera, N., Finetti, P., Sircoulomb, F., Rouge, C., Lasorsa, L., Letessier, A., Ginestier, C., Monville, F., et al. (2005). Comprehensive profiling of 8p11-12 amplification in breast cancer. Mol Cancer Res 3, 655-667.
Gianni, L., Zambetti, M., Clark, K., Baker, J., Cronin, M., Wu, J., Mariani, G., Rodriguez, J., Carcangiu, M., Watson, D., et al. (2005). Gene Expression Profiles in Paraffin-Embedded Core Biopsy Tissue Predict Response to Chemotherapy in Women With Locally Advanced Breast Cancer. J Clin Oncol.
Greten, F. R., and Karin, M. (2004). The IKK/NF-kappaB activation pathway-a target for prevention and treatment of cancer. Cancer Lett 206, 193-199.
Hackett, C. S., Hodgson, J. G., Law, M. E., Fridlyand, J., Osoegawa, K., de Jong, P. J., Nowak, N.J., Pinkel, D., Albertson, D. G., Jain, A., et al. (2003). Genome-wide array CGH analysis of murine neuroblastoma reveals distinct genomic aberrations which parallel those in human tumors. Cancer Res 63, 5266-5273.
Hanahan, D., and Weinberg, R. A. (2000). The hallmarks of cancer. Cell 100, 57-70.
Hartigan, J. A. (1975). Clustering Algorithms (New York: Wiley).
Hinds, P. W., Dowdy, S. F., Eaton, E. N., Arnold, A., and Weinberg, R. A. (1994). Function of a human cyclin gene as an oncogene. Proc Natl Acad Sci USA 91, 709-713.
Hodgson, G., Hager, J. H., Vole, S., Hariono, S., Wernick, M., Moore, D., Nowak, N., Albertson, D. G., Pinkel, D., Collins, C., et al. (2001). Genome scanning with array CGH delineates regional alterations in mouse islet carcinomas. Nat Genet 29, 459-464.
Huang, G., Krig, S., Kowbel, D., Xu, H., Hyun, B., Volik, S., Feuerstein, B., Mills, G. B., Stokoe, D., Yaswen, P., and Collins, C. (2005). ZNF217 suppresses cell death associated with chemotherapy and telomere dysfunction. Hum Mol Genet 14, 3219-3225.
Hyman, E., Kauraniemi, P., Hautaniemi, S., Wolf, M., Mousses, S., Rozenblum, E., Ringner, M., Sauter, G., Monni, O., Elkahloun, A., et al. (2002). Impact of DNA amplification on gene expression patterns in breast cancer. Cancer Res 62, 6240-6245.
Irizarry, R., Bolstad, B., Collin, F., Cope, L., Hobbs, B., and Speed, T. (2003). Summaries of Affymetrix GeneChip probe level data. Nucleic Acids Research 31, el 5.
Isola, J., Chu, L., DeVries, S., Matsumura, K., Chew, K., Ljung, B. M., and Waldman, F. M. (1999). Genetic alterations in ERBB2-amplified breast carcinomas. Clin Cancer Res 5, 4140-4145.
Isola, J. J., Kallioniemi, O. P., Chu, L. W., Fuqua, S. A., Hilsenbeck, S. G., Osborne, C. K., and Waldman, F. M. (1995). Genetic aberrations detected by comparative genomic hybridization predict outcome in node-negative breast cancer. Am J Pathol 147, 905-911.
Jain, A. N., Chin, K., Borresen-Dale, A. L., Erikstein, B. K., Eynstein Lonning, P., Kaaresen, R., and Gray, J. W. (2001). Quantitative analysis of chromosomal CGH in human breast tumors associates copy number abnormalities with p53 status and patient survival. Proc Natl Acad Sci USA 98, 7952-7957.
Jain, A. N., Tokuyasu, T. A., Snijders, A. M., Segraves, R., Albertson, D. G., and Pinkel, D. (2002). Fully automatic quantification of microarray image data. Genome Res 12, 325-332.
Jones, P. A. (2005). Overview of cancer epigenetics. Semin Hematol 42, S3-8.
Kallioniemi, A., Kallioniemi, O. P., Piper, J., Tanner, M., Stokke, T., Chen, L., Smith, H. S., Pinkel, D., Gray, J. W., and Waldman, F. M. (1994). Detection and mapping of amplified DNA sequences in breast cancer by comparative genomic hybridization. Proc Natl Acad Sci USA 91, 2156-2160.
Kallioniemi, O. P., Kallioniemi, A., Kurisu, W., Thor, A., Chen, L. C., Smith, H. S., Waldman, F. M., Pinkel, D., and Gray, J. W. (1992). ERBB2 amplification in breast cancer analyzed by fluorescence in situ hybridization. Proc Natl Acad Sci USA 89, 5321-5325.
Kauraniemi, P., Barlund, M., Monni, O., and Kallioniemi, A. (2001). New amplified and highly expressed genes discovered in the ERBB2 amplicon in breast cancer by cDNA microarrays. Cancer Res 61, 8235-8240.
Kauraniemi, P., Kuukasjarvi, T., Sauter, G., and Kallioniemi, A. (2003). Amplification of a 280-kilobase core region at the ERBB2 locus leads to activation of two hypothetical proteins in breast cancer. Am J Pathol 163, 1979-1984.
Knuutila, S., Autio, K., and Aalto, Y. (2000). Online access to CGH data of DNA sequence copy number changes. Am J Pathol 157, 689.
Lam, L. T., Davis, R. E., Pierce, J., Hepperle, M., Xu, Y., Hottelet, M., Nong, Y., Wen, D., Adams, J., Dang, L., and Staudt, L. M. (2005). Small molecule inhibitors of IkappaB kinase are selectively toxic for subgroups of diffuse large B-cell lymphoma defined by gene expression profiling. Clin Cancer Res 11, 28-40.
Loo, L. W., Grove, D. I., Williams, E. M., Neal, C. L., Cousens, L. A., Schubert, E. L., Holcomb, I. N., Massa, H. F., Glogovac, J., Li, C. I., et al. (2004). Array comparative genomic hybridization analysis of genomic alterations in breast cancer subtypes. Cancer Res 64, 8541-8549.
Mazzocca, A., Coppari, R., De Franco, R., Cho, J. Y., Libermann, T. A., Pinzani, M., and Toker, A. (2005). A secreted form of ADAM9 promotes carcinoma invasion through tumor-stromal interactions. Cancer Res 65, 4728-4738.
Naylor, T. L., Greshock, J., Wang, Y., Colligon, T., Yu, Q. C., Clemmer, V., Zaks, T. Z., and Weber, B. L. (2005). High resolution genomic analysis of sporadic breast cancer using array-based comparative genomic hybridization. Breast Cancer Res 7, R1186-1198.
Nonet, G., Stampfer, M., Chin, K., Gray, J. W., Collins, C., and Yaswen, P. (2001). The ZNF217 gene amplified in breast cancers promotes immortalization of human mammary epithelial cells. Cancer Research 61, 1250-1254.
Okunieff, P., Fenton, B. M., Zhang, L., Kern, F. G., Wu, T., Greg, J. R., and Ding, I. (2003). Fibroblast growth factors (FGFS) increase breast tumor growth rate, metastases, blood flow, and oxygenation without significant change in vascular density. Adv Exp Med Biol 530, 593-601.
Olshen, A. B., Venkatraman, E. S., Lucito, R., and Wigler, M. (2004). Circular binary segmentation for the analysis of array-based DNA copy number data. Biostatistics 5, 557-572.
Ouspenski, II, Elledge, S. J., and Brinkley, B. R. (1999). New yeast genes important for chromosome integrity and segregation identified by dosage effects on genome stability. Nucleic Acids Res 27, 3001-3008.
Perou, C. M., Jeffrey, S. S., van de Rijn, M., Rees, C. A., Eisen, M. B., Ross, D. T., Pergamenschikov, A., Williams, C. F., Zhu, S. X., Lee, J. C., et al. (1999). Distinctive gene expression patterns in human mammary epithelial cells and breast cancers. Proc Natl Acad Sci USA 96, 9212-9217.
Perou, C. M., Sorlie, T., Eisen, M. B., van de Rijn, M., Jeffrey, S. S., Rees, C. A., Pollack, J. R., Ross, D. T., Johnsen, H., Akslen, L. A., et al. (2000). Molecular portraits of human breast tumours. Nature 406, 747-752.
Pinkel, D., Segraves, R., Sudar, D., Clark, S., Poole, I., Kowbel, D., Collins, C., Kuo, W. L., Chen, C., Zhai, Y., et al. (1998). High resolution analysis of DNA copy number variation using comparative genomic hybridization to microarrays. Nat Genet 20, 207-211.
Pollack, J. R., Perou, C. M., Alizadeh, A. A., Eisen, M. B., Pergamenschikov, A., Williams, C. F., Jeffrey, S. S., Botstein, D., and Brown, P. O. (1999). Genome-wide analysis of DNA copy-number changes using cDNA microarrays. Nat Genet 23, 41-46.
Pollack, J. R., Sorlie, T., Perou, C. M., Rees, C. A., Jeffrey, S. S., Lonning, P. E., Tibshirani, R., Botstein, D., Borresen-Dale, A. L., and Brown, P. O. (2002). Microarray analysis reveals a major direct role of DNA copy number alteration in the transcriptional program of human breast tumors. Proc Natl Acad Sci USA 99, 12963-12968.
Press, M. F., Sauter, G., Bernstein, L., Villalobos, I. E., Mirlacher, M., Zhou, J. Y., Wardeh, R., Li, Y. T., Guzman, R., Ma, Y., et al. (2005). Diagnostic evaluation of HER-2 as a molecular target: an assessment of accuracy and reproducibility of laboratory testing in large, prospective, randomized clinical trials. Clin Cancer Res 11, 6598-6607.
Ramaswamy, S., Ross, K. N., Lander, E. S., and Golub, T. R. (2003). A molecular signature of metastasis in primary solid tumors. Nat Genet 33, 49-54.
Ray, M. E., Yang, Z. Q., Albertson, D., Kleer, C. G., Washburn, J. G., Macoska, J. A., and Ethier, S. P. (2004). Genomic and expression analysis of the 8p11-12 amplicon in human breast cancer cell lines. Cancer Res 64, 40-47.
Reyal, F., Stransky, N., Bernard-Pierrot, I., Vincent-Salomon, A., de Rycke, Y., Elvin, P., Cassidy, A., Graham, A., Spraggon, C., Desille, Y., et al. (2005). Visualizing chromosomes as transcriptome correlation maps: evidence of chromosomal domains containing co-expressed genes—a study of 130 invasive ductal breast carcinomas. Cancer Res 65, 1376-1383.
Russ, A. P., and Lampel, S. (2005). The druggable genome: an update. Drug Discov Today 10, 1607-1610.
Slamon, D. J., Godolphin, W., Jones, L. A., Holt, J. A., Wong, S. G., Keith, D. E., Levin, W. J., Stuart, S. G., Udove, J., Ullrich, A., and et al. (1989). Studies of the HER-2/neu proto-oncogene in human breast and ovarian cancer. Science 244, 707-712.
Snijders, A. M., Fridlyand, J., Mans, D. A., Segraves, R., Jain, A. N., Pinkel, D., and Albertson, D. G. (2003). Shaping of tumor and drug-resistant genomes by instability and selection. Oncogene 22, 4370-4379.
Snijders, A. M., Nowak, N., Segraves, R., Blackwood, S., Brown, N., Conroy, J., Hamilton, G., Hindle, A. K., Huey, B., Kimura, K., et al. (2001). Assembly of microarrays for genome-wide measurement of DNA copy number. Nat Genet 29, 263-264.
Solinas-Toldo, S., Lampel, S., Stilgenbauer, S., Nickolenko, J., Benner, A., Dohner, H., Cremer, T., and Lichter, P. (1997). Matrix-based comparative genomic hybridization: biochips to screen for genomic imbalances. Genes Chromosomes Cancer 20, 399-407.
Sorlie, T., Perou, C. M., Tibshirani, R., Aas, T., Geisler, S., Johnsen, H., Hastie, T., Eisen, M. B., van de Rijn, M., Jeffrey, S. S., et al. (2001). Gene expression patterns of breast carcinomas distinguish tumor subclasses with clinical implications. Proc Natl Acad Sci USA 98, 10869-10874.
Sorlie, T., Tibshirani, R., Parker, J., Hastie, T., Marron, J. S., Nobel, A., Deng, S., Johnsen, H., Pesich, R., Geisler, S., et al. (2003). Repeated observation of breast tumor subtypes in independent gene expression data sets. Proc Natl Acad Sci USA 100, 8418-8423.
Still, I. H., Hamilton, M., Vince, P., Wolfman, A., and Cowell, J. K. (1999). Cloning of TACC1, an embryonically expressed, potentially transforming coiled coil containing gene, from the 8p11 breast cancer amplicon. Oncogene 18, 4032-4038.
Tanaka, S., Sugimachi, K., Kawaguchi, H., Saeki, H., Ohno, S., and Wands, J. R. (2000). Grb7 signal transduction protein mediates metastatic progression of esophageal carcinoma. J Cell Physiol 183, 411-415.
Tanner, M. M., Tirkkonen, M., Kallioniemi, A., Collins, C., Stokke, T., Karhu, R., Kowbel, D., Shadravan, F., Hintz, M., Kuo, W. L., and et al. (1994). Increased copy number at 20q13 in breast cancer: defining the critical region and exclusion of candidate genes. Cancer Res 54, 4257-4260.
van 't Veer, L. J., Dai, H., van de Vijver, M. J., He, Y. D., Hart, A. A., Mao, M., Peterse, H. L., van der Kooy, K., Marton, M. J., Witteveen, A. T., et al. (2002). Gene expression profiling predicts clinical outcome of breast cancer. Nature 415, 530-536.
van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009.
Vogel, C. L., Cobleigh, M. A., Tripathy, D., Gutheil, J. C., Harris, L. N., Fehrenbacher, L., Slamon, D. J., Murphy, M., Novotny, W. F., Burchmore, M., et al. (2002). Efficacy and safety of trastuzumab as a single agent in first-line treatment of HER2-overexpressing metastatic breast cancer. J Clin Oncol 20, 719-726.
Weber-Mangal, S., Sinn, H. P., Popp, S., Klaes, R., Emig, R., Bentz, M., Mansmann, U., Bastert, G., Bartram, C. R., and Jauch, A. (2003). Breast cancer in young women (<or =35 years): Genomic aberrations detected by comparative genomic hybridization. Int J Cancer 107, 583-592.
Willenbrock, H., and Fridlyand, J. (2005). A comparison study: applying segmentation to array CGH data for downstream analyses. Bioinformatics.
Yeung, K. Y., Fraley, C., Murua, A., Raftery, A. E., and Ruzzo, W. L. (2001). Model-based clustering and data transformations for gene expression data. Bioinformatics 17, 977-987.
Yeung, K. Y., Medvedovic, M., and Bumgamer, R. E. (2004). From co-expression to co-regulation: how many microarray experiments do we need? Genome Biol 5, R48.
Yi, Y., Mirosevich, J., Shyr, Y., Matusik, R., and George, A. L., Jr. (2005). Coupled analysis of gene expression and chromosomal location. Genomics 85, 401-412.
Zhu, Y., Kan, L., Qi, C., Kanwar, Y. S., Yeldandi, A. V., Rao, M. S., and Reddy, J. K. (2000). Isolation and characterization of peroxisome proliferator-activated receptor (PPAR) interacting protein (PRIP) as a coactivator for PPAR. J Biol Chem 275, 13510-13516.

While the present sequences, compositions and processes have been described with reference to specific details of certain exemplary embodiments thereof, it is not intended that such details be regarded as limitations upon the scope of the invention. The present examples, methods, procedures, specific compounds and molecules are meant to exemplify and illustrate the invention and should in no way be seen as limiting the scope of the invention. Any patents, publications, publicly available sequences mentioned in this specification and listed above are indicative of levels of those skilled in the art to which the invention pertains and are hereby incorporated by reference to the same extent as if each were specifically and individually incorporated by reference.

TABLE 1
Univariate and multivariate associations for individual amplicons and/or disease specific survival and distant
recurrence. Also shown are the chromosomal positions of the beginning and ends of the amplicons and the flanking clones.
Associations are shown for the entire sample set and for luminal A tumors (univariate associations only).

| | | | | | | | p-value | | | |
|---|---|---|---|---|---|---|---|---|---|---|
| | | Flanking | | | p-value | | luminal A, | | p-value | |
| | Flanking | clone | | | univariate | | univariate | | multivariate | |
| Amplicon | clone (left) | (right) | kbStart | kbEnd | survival | recurrence | survival | recurrence | survival | recurrence |
| 8p11-12 | RP11- | RP11- | 33579 | 43001 | 0.011 | 0.004 | 0.022 | 0.004 | 0.037 | 0.006 |
| | 258M15 | 73M19 | | | | | | | | |
| 8q24 | RP11-65D17 | RP11- | 127186 | 132829 | 0.830 | 0.880 | 0.140 | 1.0 | 0.870 | 0.720 |
| | | 94M13 | | | | | | | | |
| 11q13-14 | CTD-2080I19 | RP11- | 68482 | 71659 | 0.540 | 0.410 | 0.016 | 0.240 | 0.660 | 0.440 |
| | | 256P19 | | | | | | | | |
| 11q13-14 | RP11- | RP11- | 73337 | 78686 | 0.230 | 0.150 | 0.016 | 0.240 | 0.360 | 0.190 |
| | 102M18 | 215H8 | | | | | | | | |
| 12q13-14 | BAL12B2624 | RP11- | 67191 | 74053 | 0.250 | 0.260 | 0.230 | 0.098 | 0.920 | 0.960 |
| | | 92P22 | | | | | | | | |
| 17q11-12 | RP11-58O8 | RP11- | 34027 | 38681 | 0.004 | 0.004 | 1.0 | 1.0 | 0.022/ 0.008 | |
| | | 87N6 | | | | | | | | |
| 17q21-24 | RP11-234J24 | RP11- | 45775 | 70598 | 0.960 | 0.920 | 0.610 | 0.290 | 0.530 | 0.630 |
| | | 84E24 | | | | | | | | |
| 20q13 | RMC20B4135 | RP11- | 51669 | 53455 | 0.340 | 0.800 | 0.048 | 0.140 | 0.590 | 0.970 |
| | | 278I13 | | | | | | | | |
| 20q13 | GS-32I19 | RP11- | 55630 | 59444 | 0.087 | 0.230 | 0.048 | 0.140 | 0.060 | 0.220 |
| | | 94A18 | | | | | | | | |
| Any | | | | | 0.005 | 0.003 | 0.024 | 0.120 | 0.034 | 0.009 |
| ammplicon | | | | | | | | | | |

TABLE 2
Associations of genomic variables with clinical features.

| | | | | Number of | Presence of |
|---|---|---|---|---|---|
| | Fraction of | Total number of | Number of | recurrent | recurrent |
| | genome altered¹ | transitions² | amplified arms³ | amplicons⁴ | amplicons⁵ |
| 1. ER (neg vs. pos) | <0.001 | <0.001 | 0.376 | 0.147 | 0.482 |
| 2. PR (neg vs. pos) | 0.005 | <0.001 | <0.050 | 0.319 | 0.390 |
| 3. Nodes (pos vs. neg) | 0.053 | 0.106 | 0.012 | 0.012 | 0.008 |
| 4. Stage (>1 vs. 1) | 0.013 | 0.052 | 0.045 | 0.312 | 0.368 |
| 5. ERBB2 (pos vs. neg) | 0.650 | 0.830 | 0.015 | <.001 | <0.001 |
| 6. Ki67 (>0.1 vs. <0.1) | 0.013 | 0.031 | 0.024 | 0.010 | 0.005 |
| 7. P53 (pos vs. neg) | 0.001 | <0.001 | 0.043 | 0.573 | 0.171 |
| 8. Size | 0.339 | 0.088 | 0.016 | 0.005 | 0.015 |
| 9. Age at Dx | 0.767 | 0.361 | 0.223 | 0.905 | 0.947 |
| 10. SBR Grade | <0.001 | <0.001 | 0.008 | 0.206 | 0.035 |
| 11. Expression subtype | <0.001 | <0.001 | 0.002 | 0.003 | <0.001 |
| 12. Genomic subtype | <0.001 | <0.001 | <0.001 | <0.001 | <0.001 |

^{1,2,3,4}Kruskal-wallis test (1-7, 11, 12), significance of robust linear regression standardized coefficient (8-10
⁵Fisher exact test (1-7, 11, 12), significance of robust linear regression standardized coefficient (8-10)

TABLE 3
Functional characteristics of genes in recurrent amplicons associated with reduced survival duration in breast cancer.
Functional annotation was based on the Human Protein Reference Database at the http address hprd.org. Genes highlighted in
dark gray are associated with reduced survival duration or distant recurrence when over expressed in non-Amplifying tumors.
Genes highlighted in light gray are significantly associated with reduced survival duration or distant recurrence (p < 0.05) when
down regulated in non-Amplifying tumors. Distances to sites of recurrent viral integration were determined from published
information (Akagi et al., 2004). The last column identifies genes having predicted protein folding characteristics suggesting that
they might bedruggable (Russ and Lampel, 2005).

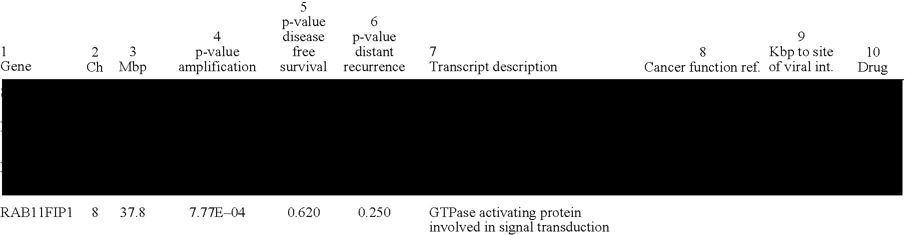

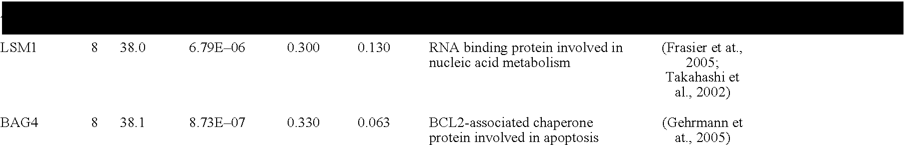

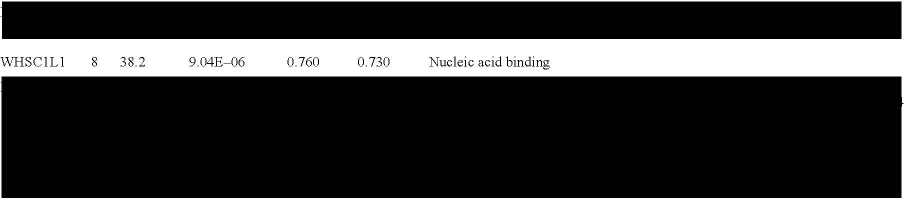

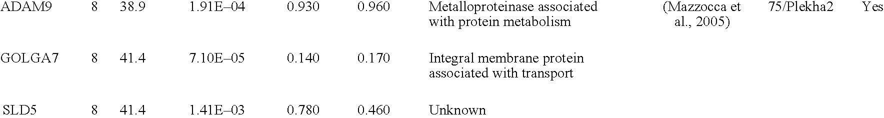

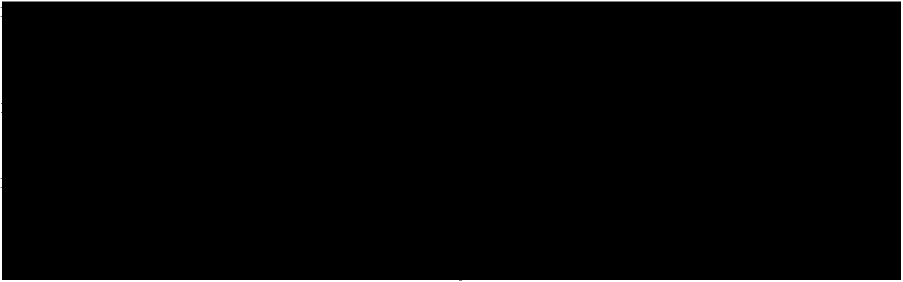

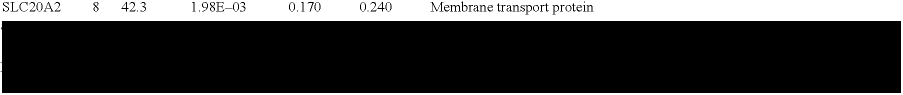

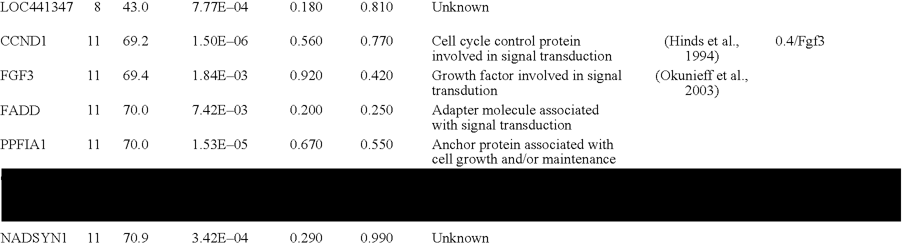

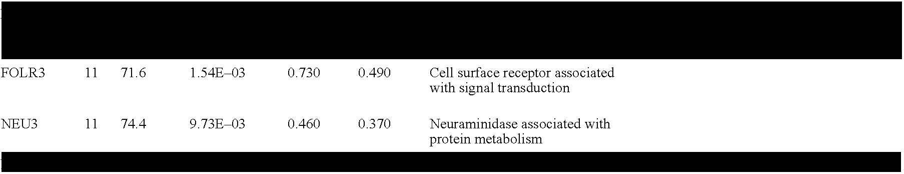

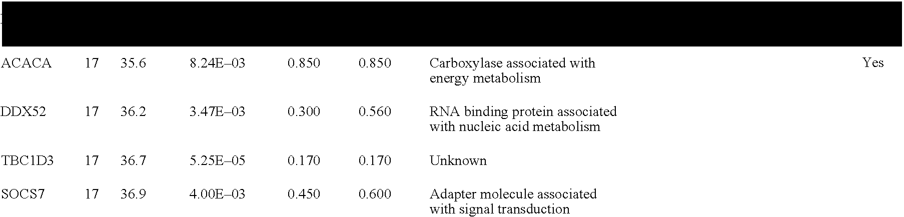

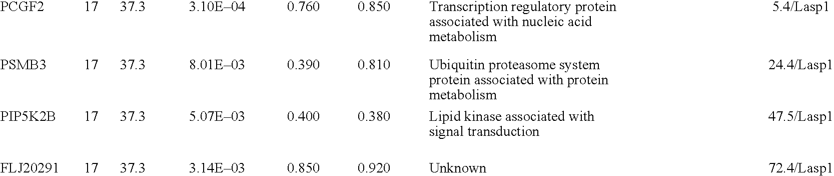

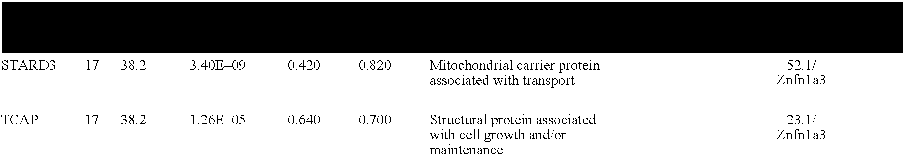

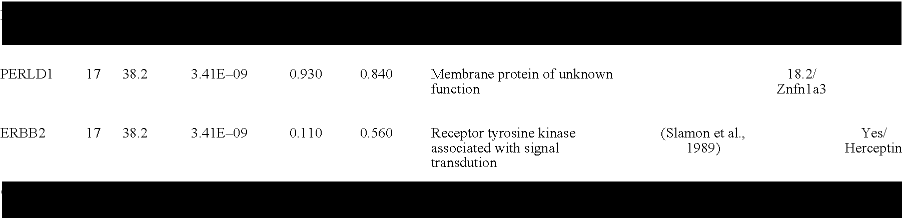

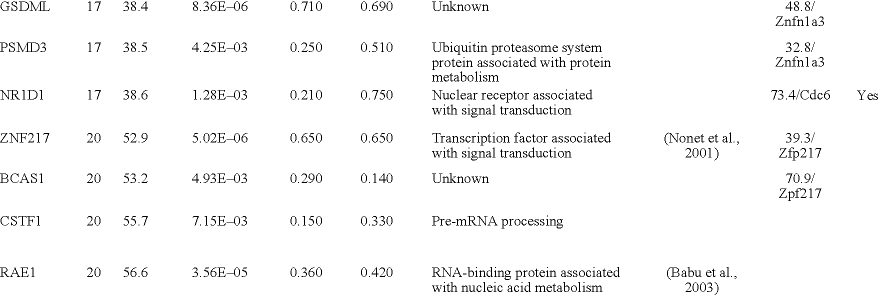

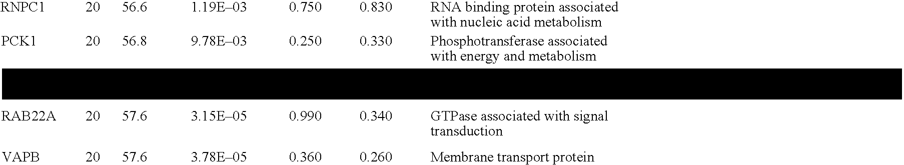

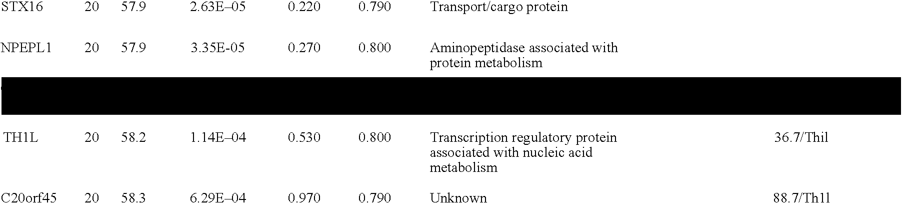

TABLE 4
Univariate p-values with the corresponding 95% confidence intervals for associations with disease-specific survival
and distant recurrence emdpoints and the corresponding multivariate results for those found to be significant in
univariate analyses (p < .05) for at least one of the clinical end points. Only variables individually
significant at p < .05 for at least one of the two end points are included in the multivariate regression.
Stage and SBR Grade are treated as continuous variables rather then factors. In each column pair, the left subcolumn
lists results for disease-specific survival and the right subcolumn lists results for time to distant recurrence.

| | | | Hazard | | | | | | Hazard | | | |
|---|---|---|---|---|---|---|---|---|---|---|---|---|
| | p-value | | ratio | | Confidence interval | | p-value | | ratio | | Confidence interval | |
| | univariate | | univariate | | univariate | | multivariate | | multivariate | | multivariate | |
| Size | <1e−03 | <1e−03 | 1.6 | 1.6 | 1.3, 2 | 1.3, 2 | 0.012 | 0.005 | 1.5 | 1.7 | 1.1, 2.1 | 1.2, 2.4 |
| Nodal status | 0.001 | 0.016 | 3.8 | 2.5 | 1.7, 8.5 | 1.2, 5.4 | 0.034 | 0.1 | 3.0 | 2.4 | 1.1, 8.7 | 0.9, 6.7 |
| Stage | <1e−03 | 0.007 | 2.9 2.3 | 1.7, 5.2 | 1.3, 4.1 | 0.690 | 0.32 | 0.6 | 0.3, 2.3 | 0.2, 1.8 | | |
| | ER | 0.29 | 0.74 | 0.7 | 0.9 | 0.3, 1.4 | 0.4, 1.8 | | | | | |
| PR | 0.14 | 0.13 | 0.6 | 0.6 | 0.3, 1.2 | 0.3, 1.2 | | | | | | |
| ERBB2 | 0.2 | 0.11 | 1.8 2.1 | 0.7, 4.4 | 0.8, 5.3 | | | | | | | |
| P53 | 0.82 | 0.07 | 1.1 | 2.1 | 0.5, 2.5 | 1, 4.5 | | | | | | |
| Ki67 | 0.64 | 0.41 | 1.2 | 1.4 | 0.5, 2.7 | 0.6, 3.4 | | | | | | |
| SBR Grade | 0.095 | 0.11 | 1.6 | 1.6 | 0.9, 2.8 | 0.9, 2.9 | | | | | | |

TABLE 5
Comparison of the association between expression subtypes and survival duration in 3 datastes. Leg-likelihood ratio
test p-value is shown for each model. Basal is the reference in all models. Multivariate models include size and
nodal status. In multivariate analyses, the first value shown in each cell is the p-value and the second is the
ratio of the medians in the compared groups

| | | | Hazard | | Confidence | | | | | | | |
|---|---|---|---|---|---|---|---|---|---|---|---|---|
| | | | ratio | | interval | | p-value | | Hazard ratio | | Confidence interval | |
| p-value | univariate | | univariate | | multivariate | | multivariate | | multivariate | | multivariate | |
| This study | 0.004 | 0.024 | | | | | 2e−05 | 1e−04 | | | | |
| Basal reference | | | 1 | 1 | | | | | 1 | 1 | | |
| ERBB2 | 0.02 | 0.008 | 3.4 | 5.1 | 1.2, 9.3 | 1.5, 116.8 | 0.49 | 0.07 | 1.5 | 3.2 | 0.5, 4.7 | 0.9, 11.6 |
| Luminal A | 0.19 | 0.45 | 0.5 | 0.6 | 0.2, 1.4 | 0.2, 2.1 | 0.1 | 0.32 | 0.4 | 0.5 | 0.1, 1.2 | 0.2, 1.9 |
| Luminal B | 0.18 | 0.88 | 0.24 | 1.1 | 0.03, 1.9 | 0.3, 4.7 | 0.1 | 0.87 | 0.2 | 0.9 | 0.02, 1.4 | 0.2, 3.9 |
| Normal-like | 0.19 | 0.70 | 0.25 | 0.7 | 0.03, 2 | 0.1, 3.7 | 0.14 | 0.63 | 0.2 | 0.7 | 0.03, 1.7 | 0.1, 3.5 |
| van de Vijver et | 0.0006 | 0.18 | | | | | | | | | | |
| al¹ | | | | | | | | | | | | |
| Basal reference | | | 1 | 1 | | | | | | | | |
| ERBB2 | 0.14 | 0.95 | 0.6 | 1 | 0.3, 1.2 | 0.5, 1.8 | | | | | | |
| Luminal A | 3.5e−05 | 0.1 | 0.3 | 0.6 | 0.2, 0.5 | 0.4, 1.1 | | | | | | |
| Luminal B | 0.23 | 0.7 | 0.6 | 1.1 | 0.3, 1.3 | 0.6, 2.3 | | | | | | |
| Normal-like | 0.01 | 0.23 | 0.3 | 0.7 | 0.2, 0.8 | 0.3, 1.4 | | | | | | |
| Sorlie et al² | 2e−06 | | | | | | | | | | | |
| Basal ref | | | 1 | | | | | | | | | |
| ERBB2 0.83 | | | 1.11 | | 0.4, 2.9 | | | | | | | |
| Luminal A | 0.001 | | .04 | | 0.005, .3 | | | | | | | |
| Luminal B | 0.27 | | 0.6 | | 0.2, 1.6 | | | | | | | |
| Normal-like 1 | | | 0 | | 0, Inf | | | | | | | |

¹van de Vijver MJ, He YD, van't Veer LJ, Dai H. Hart AA, et al. 2002) A gene-expression signature as a predictor of survival in breast cancer. N Engl J Med 347. 1999-2009.
²Sorlie T, Perou CM, Tibshirani R, Aas T, Geisler S, et al. 2001) Gene expression patterns of breast carcinomas distinguishing tumor subclasses with clinical implications. Proc Natl Acad Sci U S A 98: 10869-10874.

TABLE 6
Identities of 1432 gene transcripts showing significant associations
between genome copy numbers measured using array CGH and
transcript levels measured using Affymetrix U133A expression
arrays in 101 primary breast tumors. Data will be available through
CaBIG and a public web site.

| | Kbp | Kbp | Gene | Cor |
|---|---|---|---|---|
| Chr | Chrom | Genome | transcript | Pearson |
| 1 | 6295 | 6295 | FLJ23323 | 0.54 |
| 1 | 7731 | 7731 | PARK7 | 0.48 |
| 1 | 10231 | 10231 | DFFA | 0.46 |
| 1 | 10676 | 10676 | FRAP1 | 0.53 |
| 1 | 11750 | 11750 | MFN2 | 0.51 |
| 1 | 12047 | 12047 | VPS13D | 0.45 |
| 1 | 13394 | 13394 | PRDM2 | 0.56 |
| 1 | 15216 | 15216 | KIAA0962 | 0.48 |
| 1 | 15537 | 15537 | SPEN | 0.48 |
| 1 | 15958 | 15958 | FBXO42 | 0.53 |
| 1 | 26364 | 26364 | DHDDS | 0.47 |
| 1 | 27339 | 27339 | WASF2 | 0.49 |
| 1 | 32619 | 32619 | RBBP4 | 0.52 |
| 1 | 32744 | 32744 | YARS | 0.46 |
| 1 | 36349 | 36349 | MRPS15 | 0.46 |
| 1 | 37460 | 37460 | GNL2 | 0.54 |
| 1 | 37892 | 37892 | CGI-94 | 0.53 |
| 1 | 38718 | 38718 | RRAGC | 0.51 |
| 1 | 39210 | 39210 | BACF1 | 0.48 |
| 1 | 39440 | 39440 | PABPC4 | 0.59 |
| 1 | 39618 | 39618 | PPIE | 0.59 |
| 1 | 39720 | 39720 | TRIT1 | 0.58 |
| 1 | 40040 | 40040 | RLF | 0.64 |
| 1 | 40329 | 40329 | ZNF643 | 0.67 |
| 1 | 40500 | 40500 | RIMS3 | 0.65 |
| 1 | 40571 | 40571 | NFYC | 0.59 |
| 1 | 40859 | 40859 | CTPS | 0.51 |
| 1 | 40906 | 40906 | SCMH1 | 0.60 |
| 1 | 42561 | 42561 | LOC442610 | 0.54 |
| 1 | 42561 | 42561 | NSEP1 | 0.58 |
| 1 | 42646 | 42646 | C1orf50 | 0.61 |
| 1 | 43043 | 43043 | EBNA1BP2 | 0.47 |
| 1 | 43242 | 43242 | ELOVL1 | 0.58 |
| 1 | 43263 | 43263 | MED8 | 0.56 |
| 1 | 43322 | 43322 | KIAA0467 | 0.47 |
| 1 | 43410 | 43410 | PTPRF | 0.51 |
| 1 | 43529 | 43529 | LMLD2A | 0.62 |
| 1 | 43849 | 43849 | DPH2L2 | 0.53 |
| 1 | 43858 | 43858 | B4GALT2 | 0.45 |
| 1 | 44100 | 44100 | PRNPIP | 0.61 |
| 1 | 44511 | 44511 | FLJ0597 | 0.45 |
| 1 | 44730 | 44730 | EIF2B3 | 0.63 |
| 1 | 44891 | 44891 | UROD | 0.45 |
| 1 | 45208 | 45208 | MUTYH | 0.46 |
| 1 | 45463 | 45463 | SP192 | 0.57 |
| 1 | 53063 | 53063 | MAGOH | 0.45 |
| 1 | 54063 | 54063 | SSBP3 | 0.45 |
| 1 | 54551 | 54551 | TTC4 | 0.58 |
| 1 | 54902 | 54902 | USP24 | 0.56 |
| 1 | 61578 | 61578 | INADL | 0.51 |
| 1 | 67248 | 67248 | PAI-RBP1 | 0.47 |
| 1 | 77453 | 77453 | ZZZ3 | 0.54 |
| 1 | 84532 | 84532 | SSX2IP | 0.47 |
| 1 | 87217 | 87217 | LMO4 | 0.52 |
| 1 | 88617 | 88617 | PKN2 | 0.47 |
| 1 | 88786 | 88786 | GTF2B | 0.49 |
| 1 | 89754 | 89754 | LRRC5 | 0.50 |
| 1 | 92184 | 92184 | GLMN | 0.51 |
| 1 | 92769 | 92769 | RPL5 | 0.63 |
| 1 | 93017 | 93017 | M96 | 0.49 |
| 1 | 93807 | 93807 | ERBP | 0.48 |
| 1 | 100918 | 100918 | CGI-30 | 0.49 |
| 1 | 109055 | 109055 | SARS | 0.44 |
| 1 | 113747 | 113747 | DCLRE1B | 0.49 |
| 1 | 114239 | 114239 | TRIM33 | 0.64 |
| 1 | 114409 | 114409 | BCAS2 | 0.61 |
| 1 | 114558 | 114558 | UNR | 0.50 |
| 1 | 114614 | 114614 | FLJ21168 | 0.74 |
| 1 | 117414 | 117414 | MAN1A2 | 0.49 |
| 1 | 143265 | 143265 | PEX11B | 0.57 |
| 1 | 143341 | 143341 | POLR3C | 0.51 |
| 1 | 144520 | 144520 | BCL9 | 0.51 |
| 1 | 147088 | 147088 | CGI-143 | 0.46 |
| 1 | 147112 | 147112 | SF3B4 | 0.63 |
| 1 | 147256 | 147256 | VPS45A | 0.47 |
| 1 | 147454 | 147454 | APH1A | 0.52 |
| 1 | 147661 | 147661 | KIAA0460 | 0.52 |
| 1 | 147677 | 147677 | TARSL1 | 0.57 |
| 1 | 147677 | 147677 | TARSL1 | 0.57 |
| 1 | 147813 | 147813 | ENSA | 0.45 |
| 1 | 147835 | 147835 | GOLPH3L | 0.51 |
| 1 | 148116 | 148116 | SETDB1 | 0.49 |
| 1 | 148355 | 148355 | SCNM1 | 0.48 |
| 1 | 148366 | 148366 | TCFL1 | 0.59 |
| 1 | 148481 | 148481 | PIK4CB | 0.53 |
| 1 | 148592 | 148592 | POGZ | 0.56 |
| 1 | 148801 | 148801 | SNX27 | 0.57 |
| 1 | 148949 | 148949 | MRPL9 | 0.51 |
| 1 | 153241 | 153241 | MAPBPIP | 0.55 |
| 1 | 153383 | 153383 | KIAA0446 | 0.47 |
| 1 | 153400 | 153400 | PMF1 | 0.46 |
| 1 | 153909 | 153909 | FLJ12671 | 0.52 |
| 1 | 153924 | 153294 | MRPL24 | 0.55 |
| 1 | 153954 | 153954 | PRCC | 0.54 |
| 1 | 154122 | 154122 | ARGHEF11 | 0.44 |
| 1 | 157392 | 157392 | PEA15 | 0.49 |
| 1 | 157463 | 157463 | PEX19 | 0.54 |
| 1 | 157476 | 157476 | COPA | 0.49 |
| 1 | 157530 | 157530 | NCSTN | 0.55 |
| 1 | 158287 | 158287 | PFDN2 | 0.47 |
| 1 | 158305 | 158305 | NIT1 | 0.49 |
| 1 | 158306 | 158306 | DEDD | 0.50 |
| 1 | 158340 | 158340 | Ufc1 | 0.57 |
| 1 | 158346 | 158346 | USP21 | 0.56 |
| 1 | 158353 | 158353 | PPOX | 0.46 |
| 1 | 158358 | 158358 | B4GALT3 | 0.65 |
| 1 | 158386 | 158386 | NDUFS2 | 0.64 |
| 1 | 158501 | 158501 | SDHC | 0.56 |
| 1 | 158907 | 158907 | DUSP12 | 0.53 |
| 1 | 158923 | 158923 | ATF6 | 0.52 |
| 1 | 159719 | 159719 | UAP1 | 0.54 |
| 1 | 162819 | 162819 | ALDH9A1 | 0.45 |
| 1 | 162884 | 162884 | LOC54499 | 0.53 |
| 1 | 163996 | 163996 | POGK | 0.54 |
| 1 | 166525 | 166525 | BLZF1 | 0.44 |
| 1 | 166949 | 166949 | MGC9064 | 0.45 |
| 1 | 167010 | 167010 | SCYL3 | 0.53 |
| 1 | 168721 | 168721 | BAT2D1 | 0.47 |
| 1 | 168901 | 168909 | VAMP4 | 0.51 |
| 1 | 168990 | 168990 | KIAA0859 | 0.66 |
| 1 | 169650 | 169650 | PIGC | 0.61 |
| 1 | 170923 | 170923 | KLHL20 | 0.68 |
| 1 | 172209 | 172209 | CACYBP | 0.48 |
| 1 | 172223 | 172223 | MRPS14 | 0.57 |
| 1 | 172365 | 172365 | KIAA0040 | 0.47 |
| 1 | 177071 | 177071 | LOC163590 | 0.46 |
| 1 | 177091 | 177091 | LAP18 | 0.52 |
| 1 | 178182 | 178182 | STX6 | 0.56 |
| 1 | 181899 | 181899 | C1orf22 | 0.45 |
| 1 | 183522 | 183522 | TPR | 0.57 |
| 1 | 183584 | 183854 | C1orf27 | 0.447 |
| 1 | 190317 | 190317 | SSA2 | 0.49 |
| 1 | 197664 | 197664 | ZNF281 | 0.50 |
| 1 | 198107 | 198107 | KIAA1078 | 0.48 |
| 1 | 199087 | 199087 | IPO9 | 0.53 |
| 1 | 199240 | 199240 | RMPEP | 0.53 |
| 1 | 199985 | 199985 | JARD1B | 0.55 |
| 1 | 200136 | 200136 | RABIF | 0.65 |
| 1 | 200198 | 200198 | ADIPOR1 | 0.58 |
| 1 | 200281 | 200281 | C1ord37 | 0.51 |
| 1 | 201008 | 201008 | SNRPE | 0.50 |
| 1 | 203849 | 203849 | LGTN | 0.61 |
| 1 | 205009 | 205009 | MCP | 0.57 |
| 1 | 207038 | 207038 | IRF56 | 0.51 |
| 1 | 207081 | 207081 | MGC29875 | 0.58 |
| 1 | 208513 | 208513 | RCOR3 | 0.48 |
| 1 | 208998 | 208998 | LPGAT1 | 0.46 |
| 1 | 209663 | 209663 | SCIRP10 | 0.47 |
| 1 | 210225 | 210225 | LOC90806 | 0.55 |
| 1 | 210281 | 210281 | RPS6KC1 | 0.45 |
| 1 | 212797 | 212797 | KCTD3 | 0.58 |
| 1 | 215515 | 215515 | CGI-115 | 0.46 |
| 1 | 217330 | 217330 | FLJ10326- | 0.55 |
| 1 | 217380 | 217380 | RAB3- | 0.59 |
| | | | GAP150 | |
| 1 | 217978 | 217978 | FLJ20605 | 0.50 |
| 1 | 221276 | 221276 | FBXO28 | 0.54 |
| 1 | 221346 | 221346 | DEGS1 | 0.52 |
| 1 | 221390 | 221390 | NVL | 0.51 |
| 1 | 221519 | 221519 | HSPC163 | 0.49 |
| 1 | 221550 | 221550 | WDR26 | 0.45 |
| 1 | 223225 | 223225 | H3F3A | 0.54 |
| 1 | 223307 | 223307 | ACBD3 | 0.61 |
| 1 | 225245 | 225245 | ARF1 | 0.52 |
| 1 | 225263 | 225263 | C1orf35 | 0.47 |
| 1 | 225303 | 225303 | GUK1 | 0.49 |
| 1 | 226368 | 226368 | RAB4A | 0.53 |
| 1 | 226402 | 226402 | APHAR | 0.50 |
| 1 | 226538 | 226538 | NUP133 | 0.48 |
| 1 | 228412 | 228412 | GNPAT | 0.55 |
| 1 | 228412 | 228412 | GNPAT | 0.55 |
| 1 | 232533 | 232533 | GGPS1 | 0.55 |
| 1 | 232572 | 232572 | TBCE | 0.55 |
| 1 | 233755 | 233755 | FLJ10359 | 0.49 |
| 1 | 241519 | 241519 | ADSS | 0.48 |
| 1 | 243651 | 243651 | TFB2M | 0.47 |
| 1 | 245947 | 245947 | ZNF672 | 0.51 |
| 2 | 9651 | 255779 | ADAM17 | 0.44 |
| 2 | 9651 | 255779 | LOC285148 | 0.50 |
| 2 | 9746 | 255874 | YWHAG | 0.54 |
| 2 | 15329 | 261457 | NAG | 0.64 |
| 2 | 15754 | 261881 | DDX1 | 0.68 |
| 2 | 16718 | 252846 | FAM49A | 0.51 |
| 2 | 28945 | 285073 | SFRS7 | 0.45 |
| 2 | 64556 | 310784 | HSPC159 | 0.49 |
| 2 | 85818 | 331946 | USP39 | 0.45 |
| 2 | 96486 | 324614 | BRRN1 | 0.47 |
| 2 | 172051 | 418179 | TLK1 | 0.44 |
| 2 | 238888 | 485016 | LRRFIP1 | 0.56 |
| 3 | 3167 | 492911 | CRBN | 0.52 |
| 3 | 4320 | 494064 | SETMAR | 0.45 |
| 3 | 5139 | 494883 | ARL10C | 0.52 |
| 3 | 10318 | 500062 | SEC13L1 | 0.46 |
| 3 | 12574 | 502317 | MKRN2 | 0.51 |
| 3 | 12600 | 502344 | RAF1 | 0.49 |
| 3 | 13333 | 503077 | NUP210 | 0.52 |
| 3 | 14162 | 503906 | XPC | 0.67 |
| 3 | 14964 | 504708 | NR2C2 | 0.52 |
| 3 | 38041 | 527785 | ACAA1 | 0.45 |
| 3 | 39054 | 528798 | WDR48 | 0.49 |
| 3 | 39413 | 529157 | RPSA | 0.46 |
| 3 | 40459 | 530203 | RPL14 | 0.50 |
| 3 | 44978 | 534722 | EXOSC7 | 0.47 |
| 3 | 48748 | 538942 | PRKAR2A | 0.59 |
| 3 | 48919 | 538663 | ARIH2 | 0.45 |
| 3 | 49026 | 538770 | FLJ20259 | 0.44 |
| 3 | 49092 | 538836 | QARS | 0.52 |
| 3 | 50566 | 540310 | HEMK1 | 0.45 |
| 3 | 51976 | 541720 | ACY1 | 0.45 |
| 3 | 51986 | 541730 | RPL29 | 0.45 |
| 3 | 52247 | 541991 | PRO2730 | 0.47 |
| 3 | 52393 | 542137 | BAP1 | 0.46 |
| 3 | 53279 | 543023 | DCP1A | 0.52 |
| 3 | 72347 | 562091 | RYBP | 0.50 |
| 3 | 72719 | 562463 | SHQ1 | 0.62 |
| 3 | 109643 | 599386 | DZIP3 | 0.59 |
| 3 | 114759 | 604503 | MAK3 | 0.49 |
| 3 | 114787 | 604531 | ATP6V1A | 0.50 |
| 3 | 127477 | 617221 | MGC11349 | 0.46 |
| 3 | 128613 | 618357 | GPR175 | 0.49 |
| 3 | 129092 | 618836 | SEC61A1 | 0.56 |
| 3 | 129660 | 619404 | RPN1 | 0.53 |
| 3 | 129766 | 619510 | RAB7 | 0.54 |
| 3 | 130319 | 620062 | DC12 | 0.49 |
| 3 | 130471 | 620215 | MBD4 | 0.55 |
| 3 | 130690 | 520434 | TMCC1 | 0.49 |
| 3 | 135197 | 624941 | RYK | 0.48 |
| 3 | 135835 | 625579 | EPHB1 | 0.54 |
| 3 | 137005 | 626749 | PPP2R3A | 0.58 |
| 3 | 137902 | 627646 | NCK1 | 0.47 |
| 3 | 139534 | 629278 | Cap70 | 0.45 |
| 3 | 140384 | 630128 | MRPS22 | 0.55 |
| 3 | 142010 | 631754 | FLJ10618 | 0.54 |
| 3 | 14401 | 633785 | SR140 | 0.48 |
| 3 | 150030 | 639774 | GYG | 0.56 |
| 3 | 150557 | 640301 | WWTR1 | 0.57 |
| 3 | 160312 | 650056 | SCHIP1 | 0.44 |
| 3 | 181951 | 671695 | FXR1 | 0.56 |
| 3 | 185194 | 674938 | DVL3 | 0.49 |
| 3 | 187585 | 677329 | FLJ10560 | 0.46 |
| 3 | 197793 | 687537 | PAK2 | 0.58 |
| 3 | 197988 | 687731 | SENP5 | 0.59 |
| 3 | 197989 | 687733 | NCBP2 | 0.57 |
| 3 | 198098 | 687842 | DLG1 | 0.54 |
| 3 | 198725 | 688469 | KIAA0226 | 0.47 |
| 4 | 1195 | 690283 | CTBP1 | 0.48 |
| 4 | 1946 | 691034 | WHSC2 | 0.49 |
| 4 | 2659 | 691747 | C4ord8 | 0.59 |
| 4 | 2775 | 691863 | TNIP2 | 0.49 |
| 4 | 2877 | 691965 | ADD1 | 0.61 |
| 4 | 2964 | 692052 | TETRAN | 0.47 |
| 4 | 4486 | 692574 | STX18 | 0.56 |
| 4 | 54159 | 743247 | FIP1L1 | 0.60 |
| 4 | 56178 | 745266 | TPARL | 0.46 |
| 4 | 56214 | 745302 | CLOCK | 0.65 |
| 4 | 68488 | 757576 | FLJ10808 | 0.55 |
| 4 | 69182 | 758270 | YT521 | 0.53 |
| 4 | 69746 | 758834 | TMPRSS11E | 0.52 |
| 4 | 72020 | 761108 | SAS10 | 0.61 |
| 4 | 72054 | 761142 | LOC441022 | 0.59 |
| 4 | 72054 | 761142 | RIPX | 0.49 |
| 4 | 72152 | 761239 | GRSF1 | 0.54 |
| 4 | 72326 | 761414 | DCK | 0.44 |
| 4 | 76865 | 765953 | RCHY1 | 0.44 |
| 4 | 77026 | 766114 | G3BP2 | 0.55 |
| 4 | 77108 | 766196 | VDP | 0.54 |
| 4 | 77329 | 766417 | SDAD1 | 0.60 |
| 4 | 83733 | 772821 | HNRPD | 0.47 |
| 4 | 101279 | 790367 | FLJ14281 | 0.44 |
| 4 | 104176 | 793264 | UBE2D3 | 0.54 |
| 4 | 140556 | 8229644 | ELF2 | 0.45 |
| 4 | 140789 | 829877 | NDUFC1 | 0.46 |
| 4 | 140800 | 8229888 | NARG1 | 0.48 |
| 4 | 142720 | 831808 | ZNF330 | 0.45 |
| 4 | 185122 | 874210 | ING2 | 0.45 |
| 5 | 271 | 881091 | SDHA | 0.45 |
| 5 | 496 | 881316 | SEC6L1 | 0.46 |
| 5 | 946 | 881766 | TRIP13 | 0.47 |
| 5 | 1514 | 882334 | FLJ12443 | 0.48 |
| 5 | 1854 | 882674 | NDUFS6 | 0.49 |
| 5 | 31447 | 912267 | RNASE3L | 0.69 |
| 5 | 31578 | 912398 | FLJ11193 | 0.47 |
| 5 | 32273 | 913093 | MTMR12 | 0.54 |
| 5 | 32273 | 913093 | MTMR12 | 0.54 |
| 5 | 37022 | 917842 | NIPBL | 0.46 |
| 5 | 37152 | 917972 | FLJ13231 | 0.50 |
| 5 | 37425 | 918245 | FLJ10233 | 0.57 |
| 5 | 43169 | 923989 | ZNF131 | 0.49 |
| 5 | 66426 | 947246 | MAST4 | 0.46 |
| 5 | 77740 | 958560 | SCAMP1 | 0.45 |
| 5 | 80800 | 961620 | SSBP2 | 0.54 |
| 5 | 80800 | 961620 | 0.54 | |
| 5 | 118864 | 999684 | HSD17B4 | 0.45 |
| 5 | 131782 | 1012602 | SLC22A5 | 0.52 |
| 5 | 133938 | 1014757 | PHF15 | 0.46 |
| 5 | 134150 | 1014970 | CAMLG | 0.50 |
| 5 | 134746 | 1015566 | H2AFY | 0.48 |
| 5 | 139934 | 1020754 | ANKHD1 | 0.49 |
| 5 | 14909 | 1030229 | KIAA0194 | 0.48 |
| 5 | 179091 | 1059911 | RUFY1 | 0.50 |
| 5 | 179270 | 1060089 | MAML1 | 0.60 |
| 5 | 171399 | 1060219 | KIAA0676 | 0.51 |
| 6 | 10488 | 1072343 | PAK1P1 | 0.47 |
| 6 | 20510 | 1062365 | E2F3 | 0.62 |
| 6 | 24532 | 1086387 | MRS2L | 0.51 |
| 6 | 24775 | 1086630 | THEM2 | 0.48 |
| 6 | 24883 | 1086738 | GMNN | 0.54 |
| 6 | 25705 | 1088560 | ABT1 | 0.45 |
| 6 | 31738 | 1093593 | CSNK2B | 0.51 |
| 6 | 32024 | 1093879 | RDBP | 0.51 |
| 6 | 36042 | 1097897 | MAPK14 | 0.49 |
| 6 | 36509 | 1098363 | STK38 | 0.49 |
| 6 | 41936 | 1103791 | BYSL | 0.45 |
| 6 | 43029 | 1104884 | KLHDC3 | 0.54 |
| 6 | 43450 | 1105305 | ABCC10 | 0.45 |
| 6 | 71373 | 1133228 | SMAP1 | 0.55 |
| 6 | 75943 | 1137798 | COXA7A2 | 0.48 |
| 6 | 76308 | 1138163 | SENP6 | 0.54 |
| 6 | 83773 | 1145626 | KIAA1117 | 0.50 |
| 6 | 87967 | 1149821 | ZNF292 | 0.45 |
| 6 | 88042 | 1149897 | C6orf62 | 0.447 |
| 6 | 89316 | 1151170 | RNGTT | 0.46 |
| 6 | 90349 | 1152204 | MDN1 | 0.46 |
| 6 | 91190 | 1153045 | MAP3K7 | 0.45 |
| 6 | 99894 | 1161749 | C6orf111 | 0.48 |
| 6 | 105771 | 1167626 | PREP | 0.64 |
| 6 | 106678 | 1168533 | APG5L | 0.73 |
| 6 | 107123 | 1168978 | QRSL1 | 0.61 |
| 6 | 107519 | 1169374 | C6orf210 | 0.59 |
| 6 | 106260 | 1170115 | SEC63 | 0.69 |
| 6 | 108522 | 1170377 | SNX3 | 0.52 |
| 6 | 106927 | 1170782 | FOXO3A | 0.60 |
| 6 | 109829 | 1171684 | ZBTB24 | 0.57 |
| 6 | 110547 | 1172402 | CDC40 | 0.55 |
| 6 | 110977 | 1172832 | CDC2L6 | 0.55 |
| 6 | 111242 | 1173096 | AMD1 | 0.51 |
| 6 | 111666 | 1173521 | REV3L | 0.48 |
| 6 | 111864 | 1173719 | RAF3IP2 | 0.61 |
| 6 | 114307 | 1176162 | HDAC2 | 0.58 |
| 6 | 119180 | 1181035 | C6orf61 | 0.66 |
| 6 | 119203 | 1181057 | ASF1A | 0.63 |
| 6 | 119262 | 1181116 | C6orf60 | 0.55 |
| 6 | 125577 | 1187432 | C6orf74 | 0.54 |
| 6 | 131876 | 1193731 | CRSP3 | 0.59 |
| 6 | 132762 | 1194617 | STX7 | 0.62 |
| 6 | 134255 | 1196110 | TBPL1 | 0.53 |
| 6 | 135219 | 1197074 | ALDH8A1 | 0.45 |
| 6 | 135266 | 1197121 | HBS1L | 0.59 |
| 6 | 138706 | 1200561 | HEBP2 | 0.57 |
| 6 | 143753 | 1205608 | PEX3 | 0.49 |
| 6 | 145927 | 1207782 | EPM2A | 0.52 |
| 6 | 160299 | 1222154 | IGF2R | 0.49 |
| 6 | 170701 | 1232556 | PSMB1 | 0.47 |
| 6 | 170743 | 1232598 | PDCD2 | 0.45 |
| 7 | 2019 | 1234788 | FTSJ2 | 0.49 |
| 7 | 2139 | 1234908 | EIF3S9 | 0.46 |
| 7 | 2188 | 1234957 | CHST12 | 0.45 |
| 7 | 2343 | 1235113 | IQCE | 0.49 |
| 7 | 5808 | 1238577 | EIF2AK1 | 0.50 |
| 7 | 6607 | 1239376 | C7orf28B | 0.44 |
| 7 | 7352 | 1240121 | FLJ203223 | 0.54 |
| 7 | 7898 | 1240667 | ICA1 | 0.51 |
| 7 | 27522 | 1260291 | TAX1BP1 | 0.53 |
| 7 | 43412 | 1276181 | FLJ10803 | 0.49 |
| 7 | 43656 | 1276426 | URG4 | 0.54 |
| 7 | 44164 | 1276933 | NUDCD3 | 0.59 |
| 7 | 44346 | 1277116 | DDX56 | 0.56 |
| 7 | 55861 | 1288631 | OCT6A | 0.45 |
| 7 | 72129 | 1304899 | NSUN5 | 0.53 |
| 7 | 72129 | 1204899 | WBSCR20B | 0.47 |
| 7 | 72267 | 1205036 | BAZ1B | 0.47 |
| 7 | 72363 | 1305132 | BCL7B | 0.47 |
| 7 | 72510 | 1305279 | WBSCR20C | 0.50 |
| 7 | 86567 | 1319336 | TP53AP1 | 0.53 |
| 7 | 91689 | 1324458 | GATAD1 | 0.53 |
| 7 | 95930 | 1328699 | SHFM1 | 0.47 |
| 7 | 98088 | 1330857 | TRRAP | 0.50 |
| 7 | 98605 | 1331374 | PSAP1 | 0.51 |
| 7 | 98618 | 1331387 | G10 | 0.59 |
| 7 | 98628 | 1331398 | PTCD1 | 0.60 |
| 7 | 98667 | 1331437 | ATP5J2 | 0.56 |
| 7 | 98667 | 1331437 | ATP5J2 | 0.56 |
| 7 | 98714 | 1331483 | ZFP95 | 0.49 |
| 7 | 99822 | 1332591 | MOSPD3 | 0.57 |
| 7 | 99829 | 1332599 | TFR2 | 0.49 |
| 7 | 99915 | 1332685 | POP7 | 0.47 |
| 7 | 100012 | 1332781 | EPHB4 | 0.61 |
| 7 | 100062 | 1332831 | SLC12A9 | 0.52 |
| 7 | 100422 | 1333191 | ZNHIT1 | 0.62 |
| 7 | 101597 | 1334367 | PRKRIP1 | 0.50 |
| 7 | 101674 | 1334444 | POLR2J | 0.47 |
| 7 | 102498 | 1335268 | PMPCB | 0.48 |
| 1 | 102513 | 1335283 | ZRF1 | 0.65 |
| 7 | 103327 | 1336097 | ORC5L | 0.54 |
| 7 | 104733 | 1337503 | RINT-1 | 0.52 |
| 7 | 128057 | 1360826 | ATP6V1F | 0.47 |
| 7 | 128151 | 1360921 | TNPO3 | 0.46 |
| 7 | 138459 | 1371229 | LUC7L2 | 0.46 |
| 7 | 139560 | 1372330 | MKRN1 | 0.59 |
| 7 | 140113 | 1372883 | MRPS33 | 0.49 |
| 7 | 140663 | 1373432 | MULK | 0.55 |
| 7 | 149460 | 1382229 | REPIN1 | 0.46 |
| 7 | 150148 | 1382918 | SLC4A2 | 0.51 |
| 7 | 150165 | 1382935 | FASTK | 0.47 |
| 7 | 150301 | 1383071 | ABCF2 | 0.50 |
| 7 | 150556 | 1383325 | RHEB | 0.51 |
| 7 | 151115 | 1383884 | GALNT11 | 0.47 |
| 7 | 158547 | 1389316 | DNAJB6 | 0.45 |
| 8 | 1808 | 1393123 | ARGHEF10 | 0.47 |
| 8 | 9676 | 1400991 | TNKS | 0.57 |
| 8 | 9949 | 1401264 | MSRA | 0.57 |
| 8 | 11698 | 1403013 | FDFT1 | 0.50 |
| 8 | 17791 | 1409106 | PCM1 | 0.60 |
| 8 | 21799 | 1413114 | XPO7 | 0.48 |
| 8 | 21979 | 1413294 | RAI16 | 0.50 |
| 8 | 21986 | 1413301 | FLJ22494 | 0.49 |
| 8 | 22500 | 1413815 | BIN3 | 0.60 |
| 8 | 22900 | 1414215 | TNFRSF10B | 0.52 |
| 8 | 23126 | 1414441 | CHMP7 | 0.59 |
| 8 | 23168 | 1414482 | LOC203069 | 0.64 |
| 8 | 23312 | 1314627 | ENTPD4 | 0.44 |
| 8 | 26171 | 1417486 | PPP2R2A | 0.58 |
| 8 | 26262 | 1417577 | BNIP3L | 0.47 |
| 8 | 27371 | 1418685 | EPHX2 | 0.58 |
| 8 | 27613 | 1418928 | FLJ10853 | 0.63 |
| 8 | 48223 | 1439538 | KIAA0146 | 0.50 |
| 8 | 48923 | 1440238 | MCM4 | 0.48 |
| 8 | 48971 | 1440286 | UBE2V2 | 0.49 |
| 8 | 53585 | 1444900 | RB1CC1 | 0.46 |
| 8 | 54678 | 1445993 | ATP6V1H | 0.52 |
| 8 | 54929 | 1446244 | TCEA1 | 0.46 |
| 8 | 55098 | 1446413 | MRPL15 | 0.45 |
| 8 | 56736 | 1448051 | NCOA6IP | 0.55 |
| 8 | 57174 | 1448489 | CHCHD7 | 0.47 |
| 8 | 64175 | 1455490 | YTHDF3 | 0.49 |
| 8 | 66607 | 1457922 | CHPPR | 0.51 |
| 8 | 67391 | 1458706 | RRS1 | 0.59 |
| 8 | 73971 | 1465286 | TERF1 | 0.55 |
| 8 | 80881 | 1472196 | MRPS28 | 0.49 |
| 8 | 81448 | 1472763 | ZBTB10 | 0.45 |
| 8 | 82620 | 1473935 | IMPA1 | 0.53 |
| 8 | 82664 | 1473979 | ZFAND1 | 0.61 |
| 8 | 87443 | 1478758 | CGI-90 | 0.46 |
| 8 | 90902 | 1482217 | NBS1 | 0.50 |
| 8 | 95341 | 1486656 | RAD54B | 0.50 |
| 8 | 95818 | 1487133 | FLJ20530 | 0.56 |
| 8 | 95849 | 1487164 | CCNE2 | 0.48 |
| 8 | 97199 | 1488514 | UQCR8 | 0.59 |
| 8 | 97208 | 1488523 | CGI-12 | 0.60 |
| 8 | 97231 | 1488546 | PTDSS1 | 0.60 |
| 8 | 98658 | 1489973 | LYRIC | 0.70 |
| 8 | 98744 | 1490059 | LAPTM4B | 0.48 |
| 8 | 99011 | 1490325 | RPL30 | 0.57 |
| 8 | 99071 | 1490386 | HRSP12 | 0.62 |
| 8 | 99097 | 1490412 | POP1 | 0.63 |
| 8 | 99423 | 1490738 | STK3 | 0.59 |
| 8 | 101119 | 1492434 | POLR2K | 0.66 |
| 8 | 101127 | 1492442 | SPAG1 | 0.48 |
| 8 | 101226 | 1492541 | RNF19 | 0.49 |
| 8 | 101490 | 1492805 | LOC157567 | 0.57 |
| 8 | 101672 | 1492987 | PABPC1 | 0.63 |
| 8 | 101887 | 1493202 | YWHAZ | 0.71 |
| 8 | 102136 | 1493451 | LOC157562 | 0.75 |
| 8 | 102168 | 1493483 | LOC51123 | 0.71 |
| 8 | 102462 | 1493776 | TFCP2L3 | 0.53 |
| 8 | 103223 | 4945438 | EDD | 0.62 |
| 8 | 103797 | 1495112 | AZIN1 | 0.58 |
| 8 | 103990 | 1495305 | ATP6V1C1 | 0.56 |
| 8 | 104268 | 1495583 | FZD6 | 0.47 |
| 8 | 104367 | 1495682 | MFTC | 0.53 |
| 8 | 104384 | 1495698 | Gm83 | 0.62 |
| 8 | 109412 | 1500727 | KIAA0103 | 0.55 |
| 8 | 110509 | 1501824 | EBAG9 | 0.64 |
| 8 | 117614 | 1508929 | EIF3S3 | 0.52 |
| 8 | 117815 | 1509130 | RAD21 | 0.59 |
| 8 | 120700 | 1512015 | TAF2 | 0.53 |
| 8 | 120803 | 1512118 | DCC1 | 0.54 |
| 8 | 121365 | 1512680 | MRPL13 | 0.61 |
| 8 | 121365 | 1512680 | MRPL13 | 0.61 |
| 8 | 123984 | 1515299 | DERL1 | 0.71 |
| 8 | 124114 | 1515429 | MGC21654 | 0.63 |
| 8 | 124289 | 1515604 | ATAD2 | 0.58 |
| 8 | 124386 | 1515700 | FLJ10204 | 0.60 |
| 8 | 125420 | 1516735 | FLJ20772 | 0.68 |
| 8 | 125444 | 1516759 | RNF139 | 0.58 |
| 8 | 125968 | 1517283 | SQLE | 0.63 |
| 8 | 125993 | 1517308 | KIAA0196 | 0.52 |
| 8 | 128705 | 1520020 | MYC | 0.51 |
| 8 | 128965 | 1520280 | PVT1 | 0.66 |
| 8 | 120810 | 1522125 | FAM498 | 0.68 |
| 8 | 132981 | 1524296 | KIAA0143 | 0.60 |
| 8 | 133747 | 1525062 | PHF20L1 | 0.60 |
| 8 | 134428 | 1525743 | ST3GAL1 | 0.48 |
| 8 | 141504 | 1532819 | EIF2C2 | 0.66 |
| 8 | 141640 | 1532954 | PTK2 | 0.68 |
| 8 | 142403 | 1533718 | PTP4A3 | 0.45 |
| 8 | 143807 | 1535122 | JRK | 0.54 |
| 8 | 143872 | 1535187 | LOC51337 | 0.60 |
| 8 | 144479 | 1535794 | FLJ14129 | 0.63 |
| 8 | 144554 | 1535869 | MGC3113 | 0.54 |
| 8 | 144625 | 1535940 | ZC3HDC3 | 0.68 |
| 8 | 144746 | 1536061 | GSDMDC1 | 0.57 |
| 8 | 144767 | 1536082 | EEF1D | 0.68 |
| 8 | 144792 | 1536106 | PYCRL | 0.70 |
| 8 | 144800 | 1536115 | TSTA3 | 0.52 |
| 8 | 144837 | 1536152 | ZNF623 | 0.76 |
| 8 | 144979 | 1536294 | SCRIB | 0.78 |
| 8 | 145005 | 1536319 | SIAHBP1 | 0.82 |
| 8 | 145046 | 1536361 | EPPK1 | 0.45 |
| 8 | 145212 | 1536527 | OPLAH | 0.63 |
| 8 | 145240 | 1536554 | EXOSC4 | 0.82 |
| 8 | 145244 | 1536558 | GPAA1 | 0.70 |
| 8 | 145256 | 1536571 | CYC1 | 0.74 |
| 8 | 145260 | 1536574 | Sharpin | 0.62 |
| 8 | 145443 | 1536758 | LOC51236 | 0.72 |
| 8 | 145491 | 1536806 | BOP1 | 0.71 |
| 8 | 145520 | 1536835 | HSF1 | 0.78 |
| 8 | 145545 | 1536860 | DGAT1 | 0.53 |
| 8 | 145584 | 1536899 | FBXL6 | 0.72 |
| 8 | 145587 | 1536902 | GPR172A | 0.74 |
| 8 | 145623 | 1536938 | CPSF1 | 0.68 |
| 8 | 145643 | 1536958 | SLC39A4 | 0.59 |
| 8 | 145654 | 1536969 | VPS28 | 0.75 |
| 8 | 145680 | 1536995 | CYHR1 | 0.71 |
| 8 | 145742 | 1537057 | RECQL4 | 0.51 |
| 8 | 145748 | 1537063 | LRRC14 | 0.69 |
| 8 | 146003 | 1537318 | ZNF34 | 0.72 |
| 8 | 146020 | 1537335 | RPL8 | 0.73 |
| 8 | 146058 | 1537373 | ZNF7 | 0.76 |
| 8 | 146111 | 1537626 | ZNF250 | 0.66 |
| 8 | 146111 | 1537426 | ZNF250 | 0.66 |
| 8 | 146161 | 1537475 | ZNF16 | 0.67 |
| 8 | 146283 | 1537598 | FLJ20989 | 0.68 |
| 9 | 701 | 1538325 | ANKRD15 | 0.49 |
| 9 | 2005 | 1539629 | SMARCA2 | 0.50 |
| 9 | 2794 | 1540418 | KIAA0020 | 0.58 |
| 9 | 4670 | 1542293 | CDC37L1 | 0.61 |
| 9 | 4783 | 1542407 | RCL1 | 0.52 |
| 9 | 5348 | 1542972 | C9orf46 | 0.55 |
| 9 | 6001 | 1543625 | RANBP6 | 0.54 |
| 9 | 6748 | 1544372 | JMJD2C | 0.53 |
| 9 | 13097 | 1550720 | MPDZ | 0.50 |
| 9 | 15454 | 1553078 | PSIP1 | 0.49 |
| 9 | 19039 | 1556663 | RRAGA | 0.46 |
| 9 | 19043 | 1556667 | FAM29A | 0.58 |
| 9 | 21321 | 1558945 | KLHL9 | 0.61 |
| 9 | 21958 | 1559581 | CDKN2A | 0.45 |
| 9 | 32531 | 1570155 | TOPORS | 0.52 |
| 9 | 33912 | 1571536 | UBAP2 | 0.60 |
| 9 | 35047 | 1572670 | VCP | 0.50 |
| 9 | 35064 | 1572668 | FANCG | 0.57 |
| 9 | 25595 | 1573219 | TESK1 | 0.46 |
| 9 | 35722 | 1573346 | CREB3 | 0.50 |
| 9 | 36181 | 1573805 | CLTA | 0.49 |
| 9 | 37419 | 1575042 | GRHPR | 0.52 |
| 9 | 75249 | 1612873 | VPS13A | 0.45 |
| 9 | 90401 | 1628025 | NOLB | 0.49 |
| 9 | 97364 | 1634988 | SEC61B | 0.53 |
| 9 | 96444 | 1636066 | TEX10 | 0.45 |
| 9 | 121079 | 1658703 | RABGAP1 | 0.61 |
| 9 | 122492 | 1660116 | PSMB7 | 0.55 |
| 9 | 123008 | 1660631 | ARPC5L | 0.45 |
| 9 | 123017 | 1660640 | GOLGA1 | 0.48 |
| 9 | 123287 | 1660911 | PPP6C | 0.50 |
| 9 | 123492 | 1661115 | GAPVD1 | 0.56 |
| 9 | 123577 | 1661201 | MAPKAP1 | 0.45 |
| 9 | 126304 | 1663928 | CIZ1 | 0.46 |
| 9 | 127228 | 1664852 | DOLPP1 | 0.46 |
| 9 | 130012 | 1667635 | CSRP8 | 0.45 |
| 10 | 809 | 1674805 | KIAA0217 | 0.65 |
| 10 | 5811 | 1679807 | GDI2 | 0.46 |
| 10 | 11507 | 1685502 | USP6NL | 0.51 |
| 10 | 11768 | 1685784 | ECHDC3 | 0.59 |
| 10 | 11966 | 1685962 | UPF2 | 0.52 |
| 10 | 12176 | 1686171 | SEC61A2 | 0.52 |
| 10 | 12242 | 1686238 | C10orf7 | 0.559 |
| 10 | 12396 | 1686392 | CAMK1D | 0.48 |
| 10 | 13146 | 1687142 | OPTN | 0.54 |
| 10 | 13365 | 1687361 | SEPHS1 | 0.61 |
| 10 | 14884 | 1688880 | HSPA14 | 0.58 |
| 10 | 14954 | 1688950 | DCLRE1C | 0.49 |
| 10 | 15143 | 1689139 | RPP338 | 0.53 |
| 10 | 26991 | 1700986 | TPRT | 0.48 |
| 10 | 30606 | 1704602 | PAPD1 | 0.51 |
| 10 | 32304 | 1706300 | KIF5B | 0.48 |
| 10 | 34404 | 1706400 | PARD3 | 0.48 |
| 10 | 70061 | 1744056 | DDX21 | 0.47 |
| 10 | 70906 | 1744902 | COL13A1 | 0.46 |
| 10 | 71920 | 1745916 | SGPL1 | 0.48 |
| 10 | 74239 | 1748235 | HSGT1 | 0.54 |
| 10 | 74279 | 1748275 | TTC18 | 0.54 |
| 10 | 74337 | 1748333 | KIAA0974 | 0.56 |
| 10 | 74346 | 1748342 | DNAJC9 | 0.51 |
| 10 | 74355 | 1748351 | MRPS16 | 0.61 |
| 10 | 74480 | 1748476 | ANXA7 | 0.50 |
| 10 | 74541 | 1748537 | PPP3CB | 0.49 |
| 10 | 74851 | 1748847 | SEC24C | 0.60 |
| 10 | 74905 | 1748901 | KIAA0913 | 0.61 |
| 10 | 74906 | 1748902 | NDST2 | 0.59 |
| 10 | 74916 | 1748912 | CAMK2G | 0.64 |
| 10 | 75281 | 1749277 | ADK | 0.60 |
| 10 | 76315 | 1750311 | VDAC2 | 0.62 |
| 10 | 80420 | 1754415 | RAI17 | 0.54 |
| 10 | 80452 | 1754448 | PPIF | 0.51 |
| 10 | 81579 | 1755575 | ANXA11 | 0.51 |
| 10 | 81879 | 1755874 | TSPAN14 | 0.61 |
| 10 | 93879 | 1767874 | IDE | 0.47 |
| 10 | 97088 | 1771084 | C10orf61 | 0.44 |
| 10 | 103270 | 1777266 | C10orf76 | 0.44 |
| 10 | 105307 | 1779303 | OBFC1 | 0.51 |
| 10 | 115259 | 1789255 | DCLRE1A | 0.47 |
| 10 | 118705 | 1792700 | SLC18A2 | 0.46 |
| 10 | 123811 | 1797806 | PLEKHA1 | 0.48 |
| 10 | 126065 | 1800061 | KIAA0157 | 0.58 |
| 10 | 127099 | 1801905 | DHX32 | 0.53 |
| 11 | 918 | 1809951 | AP2A2 | 0.48 |
| 11 | 3794 | 1812827 | FRAG1 | 0.54 |
| 11 | 6596 | 1815629 | TAF10 | 0.63 |
| 11 | 10839 | 1819872 | LOC58486 | 0.53 |
| 11 | 11828 | 1820861 | USP47 | 0.57 |
| 11 | 11828 | 1820861 | USP47 | 0.57 |
| 11 | 35649 | 1844682 | TRIM44 | 0.54 |
| 11 | 36260 | 1845293 | COMMD9 | 0.60 |
| 11 | 43345 | 1852378 | TTC17 | 0.68 |
| 11 | 43667 | 1852700 | HSD17B12 | 0.55 |
| 11 | 43844 | 1852877 | DKFZ564C152 | 0.65 |
| 11 | 43885 | 1852918 | DEPC-1 | 0.63 |
| 11 | 44081 | 1853114 | EXT2 | 0.74 |
| 11 | 44545 | 1853578 | TP53I11 | 0.69 |
| 11 | 44552 | 1853585 | CD82 | 0.72 |
| 11 | 62102 | 1871135 | EEF1G | 0.46 |
| 11 | 64627 | 1873660 | ZFPL1 | 0.45 |
| 11 | 64639 | 1873672 | C11orf2 | 0.53 |
| 11 | 64725 | 1873758 | CAPN1 | 0.45 |
| 11 | 64877 | 1873910 | DPF2 | 0.51 |
| 11 | 66600 | 1875633 | RHOD | 0.49 |
| 11 | 66663 | 1875696 | FBXL11 | 0.58 |
| 11 | 66809 | 1875842 | ADRBK1 | 0.58 |
| 11 | 66941 | 1875974 | PPP1CA | 0.69 |
| 11 | 66971 | 1876004 | RPS6KB2 | 0.52 |
| 11 | 66981 | 1876014 | CORO1B | 0.59 |
| 11 | 67007 | 1876040 | FLJ21749 | 0.47 |
| 11 | 67026 | 1876059 | AIP | 0.57 |
| 11 | 67049 | 1876082 | CDK2AP2 | 0.57 |
| 11 | 67150 | 1876183 | NDIFV1 | 0.60 |
| 11 | 67205 | 1876238 | ALDH3B2 | 0.54 |
| 11 | 67573 | 1876606 | NDUFS8 | 0.67 |
| 11 | 67596 | 1876629 | CHKA | 0.48 |
| 11 | 68048 | 1877081 | C11orf23 | 0.52 |
| 11 | 69229 | 1878262 | CCND1 | 0.55 |
| 11 | 69776 | 1878809 | FADD | 0.81 |
| 11 | 69843 | 1878876 | PPFIA1 | 0.80 |
| 11 | 69971 | 1879004 | CTTN | 0.82 |
| 11 | 70042 | 11879075 | SHANK2 | 0.52 |
| 11 | 70872 | 1879905 | DHCR7 | 0.67 |
| 11 | 70891 | 1879924 | NADSYN1 | 0.71 |
| 11 | 71547 | 1770580 | DKFZP564M082 | 0.53 |
| 11 | 71730 | 1880763 | SKD3 | 0.48 |
| 11 | 72122 | 1881155 | CENTD2 | 0.46 |
| 11 | 73310 | 1882343 | E2IG2 | 0.49 |
| 11 | 73450 | 1882483 | DKFZP586P0123 | 0.52 |
| 11 | 73609 | 1882642 | PME-1 | 0.52 |
| 11 | 77054 | 1886087 | CLNS1A | 0.55 |
| 11 | 77104 | 1886137 | HBXAP | 0.67 |
| 11 | 77259 | 1886292 | PTD015 | 0.72 |
| 11 | 77506 | 1886539 | NDUFC2 | 0.49 |
| 11 | 77538 | 1886571 | ALG8 | 0.48 |
| 11 | 93839 | 1902872 | MRE11A | 0.50 |
| 11 | 94488 | 11903521 | SRP46 | 0.46 |
| 11 | 101805 | 1910838 | PORIMIN | 0.45 |
| 11 | 107418 | 1916451 | CUL5 | 0.60 |
| 11 | 108073 | 1817106 | DDX10 | 0.47 |
| 11 | 111011 | 1920044 | SNF1LK2 | 0.48 |
| 11 | 111434 | 1920467 | DLAT | 0.51 |
| 11 | 111489 | 1920522 | FLJ10726 | 0.63 |
| 11 | 111494 | 1920527 | TIMM8B | 0.46 |
| 11 | 111495 | 1920528 | SDHD | 0.53 |
| 11 | 111635 | 1920668 | PTS | 0.52 |
| 11 | 113142 | 1922175 | ZW10 | 0.61 |
| 11 | 113809 | 1922842 | RBM7 | 0.47 |
| 11 | 113848 | 1922881 | DKFZP566E144 | 0.50 |
| 11 | 116244 | 1925277 | APOA1 | 0.46 |
| 11 | 117810 | 1926843 | ATP5L | 0.56 |
| 11 | 117981 | 1927014 | ARCN1 | 0.56 |
| 11 | 116424 | 1927457 | RPS25 | 0.59 |
| 11 | 118505 | 1927538 | DPAGT1 | 0.52 |
| 11 | 119746 | 1928779 | ARHGEF12 | 0.48 |
| 11 | 123132 | 1932165 | ANF202 | 0.47 |
| 11 | 123481 | 1933114 | SPA17 | 0.47 |
| 11 | 124977 | 1934010 | EI24 | 0.61 |
| 11 | 125000 | 1934033 | ITM1 | 0.51 |
| 11 | 125301 | 1934334 | PUS3 | 0.61 |
| 11 | 125670 | 1934703 | SRPR | 0.53 |
| 11 | 125711 | 1934744 | DCPS | 0.45 |
| 11 | 130283 | 1939316 | SNX19 | 0.47 |
| 11 | 133656 | 1942689 | THY28 | 0.59 |
| 12 | 264 | 1943780 | JARID1A | 0.56 |
| 12 | 733 | 1944249 | WNK1 | 0.64 |
| 12 | 2837 | 1946353 | POXM1 | 0.61 |
| 12 | 2870 | 1946386 | TULP3 | 0.67 |
| 12 | 2939 | 1946455 | TEAD4 | 0.59 |
| 12 | 4467 | 1947983 | C12orf4 | 0.56 |
| 12 | 4570 | 1948085 | DYRK4 | 0.52 |
| 12 | 4629 | 1948145 | NDUFA9 | 0.65 |
| 12 | 6536 | 190052 | NOL1 | 0.45 |
| 12 | 6630 | 1950146 | ING4 | 0.45 |
| 12 | 6727 | 1950243 | MLF2 | 0.49 |
| 12 | 6847 | 1950363 | TPI1 | 0.51 |
| 12 | 6945 | 1950461 | PHB2 | 0.52 |
| 12 | 6950 | 1950466 | C2F | 0.47 |
| 12 | 7234 | 1950750 | PEX5 | 0.50 |
| 12 | 14831 | 1958347 | WBP11 | 0.44 |
| 12 | 32723 | 1976239 | DNM1L | 0.46 |
| 12 | 32788 | 1976304 | CGI-04 | 0.51 |
| 12 | 47510 | 1991026 | DDX23 | 0.50 |
| 12 | 47537 | 1991053 | RND1 | 0.45 |
| 12 | 48238 | 1991754 | MCRS1 | 0.49 |
| 12 | 48433 | 1991948 | TEGT | 0.47 |
| 12 | 50676 | 1994192 | ACVR1B | 0.51 |
| 12 | 52122 | 1995638 | DKFZp564J157 | 0.50 |
| 12 | 52161 | 1995677 | MAP3K12 | 0.52 |
| 12 | 52181 | 1995697 | TARPB2 | 0.52 |
| 12 | 54682 | 1998198 | SUOX | 0.48 |
| 12 | 56374 | 1999890 | OS-9 | 0.55 |
| 12 | 56449 | 1999964 | METTL1 | 0.48 |
| 12 | 62460 | 2005976 | TMEM5 | 0.51 |
| 12 | 63395 | 2006911 | GNS | 0.47 |
| 12 | 63558 | 2007074 | KIAA0984 | 0.55 |
| 12 | 63850 | 2007366 | LEMD3 | 0.48 |
| 12 | 65949 | 2009465 | TIP120A | 0.61 |
| 12 | 66338 | 2009854 | DYKR2 | 0.52 |
| 12 | 66975 | 2010491 | MDM1 | 0.59 |
| 12 | 67367 | 2010883 | NUP107 | 0.64 |
| 12 | 67426 | 2010942 | SLC35E3 | 0.57 |
| 12 | 67488 | 2011004 | MDM2 | 0.60 |
| 12 | 67525 | 2011041 | MGC5370 | 0.65 |
| 12 | 68040 | 2011556 | YEATS4 | 0.47 |
| 12 | 68266 | 2011781 | CCT2 | 0.52 |
| 12 | 68958 | 2012474 | CNOT2 | 0.52 |
| 12 | 74178 | 2017694 | HRB2 | 0.53 |
| 12 | 105254 | 2048770 | POLR3B | 0.59 |
| 12 | 106629 | 2050145 | PRDM4 | 0.45 |
| 12 | 107419 | 2050935 | SART3 | 0.47 |
| 12 | 110691 | 2054207 | FLJ39616 | 0.48 |
| 12 | 110663 | 2054379 | C12orf8 | 0.46 |
| 12 | 112036 | 2055551 | FLJ14827 | 0.48 |
| 12 | 118977 | 2062493 | GCN1L1 | 0.53 |
| 12 | 119060 | 2062576 | PXN | 0.52 |
| 13 | 22102 | 2097697 | MIPEP | 0.47 |
| 13 | 28990 | 2104584 | C13orf22 | 0.54 |
| 13 | 30889 | 2106483 | PFAAP5 | 0.45 |
| 13 | 39101 | 2114696 | MRPS31 | 0.51 |
| 13 | 39101 | 2114696 | MRPS31 | 0.51 |
| 13 | 50505 | 2126099 | NEK2 | 0.48 |
| 13 | 71128 | 2146722 | KIAA1008 | 0.51 |
| 13 | 71156 | 2146750 | C13orf24 | 0.54 |
| 13 | 77692 | 2153286 | C13orf10 | 0.48 |
| 13 | 94152 | 2169746 | UGCGL2 | 0.50 |
| 13 | 96304 | 2171899 | RANBP5 | 0.46 |
| 13 | 96802 | 2172397 | STK24 | 0.48 |
| 13 | 100947 | 2176542 | TPP2 | 0.50 |
| 13 | 108992 | 2184586 | FLJ12118 | 0.50 |
| 13 | 109065 | 2184660 | ING1 | 0.46 |
| 13 | 109466 | 2185060 | ARHGEF7 | 0.45 |
| 13 | 111087 | 2106682 | TUBGCP3 | 0.50 |
| 13 | 112187 | 2187781 | TFDP1 | 0.53 |
| 13 | 112918 | 2188513 | CDC16 | 0.64 |
| 13 | 112965 | 2188560 | UPF3A | 0.52 |
| 14 | 18802 | 2207439 | PARP2 | 0.50 |
| 14 | 19844 | 2208481 | CHD8 | 0.56 |
| 14 | 19936 | 2208573 | C14orf92 | 0.52 |
| 14 | 21226 | 2209863 | OXA1L | 0.47 |
| 14 | 29485 | 2218122 | AP4S1 | 0.52 |
| 14 | 33021 | 2221658 | SNX6 | 0.45 |
| 14 | 37726 | 2226364 | CTAGE5 | 0.57 |
| 14 | 43575 | 2232212 | FKBP3 | 0.54 |
| 14 | 48565 | 2237203 | C14orf38 | 0.47 |
| 14 | 48574 | 2237211 | SOS2 | 0.53 |
| 14 | 48703 | 2237341 | C14orf160 | 0.69 |
| 14 | 48769 | 2237406 | ATP5S | 0.60 |
| 14 | 48925 | 2237563 | MAP4K5 | 0.47 |
| 14 | 50446 | 2239084 | C14orf166 | 0.64 |
| 14 | 50771 | 2239408 | PTGER2 | 0.68 |
| 14 | 50771 | 2239408 | PTGER2 | 0.68 |
| 14 | 51099 | 2239736 | ERO1L | 0.50 |
| 14 | 51164 | 2239801 | PSMC6 | 0.63 |
| 14 | 53522 | 2242159 | C14orf32 | 0.48 |
| 14 | 66108 | 2254745 | VT11B | 0.54 |
| 14 | 68224 | 2256861 | SFRS5 | 0.46 |
| 14 | 71605 | 2260242 | PSEN1 | 0.61 |
| 14 | 72344 | 2260981 | ZNF410 | 0.63 |
| 14 | 72407 | 2261044 | COQ6 | 0.48 |
| 14 | 72517 | 2261154 | ALDH6A1 | 0.50 |
| 14 | 72742 | 2261379 | ABCD4 | 0.50 |
| 14 | 73339 | 2261976 | DLSTP | 0.52 |
| 14 | 73539 | 2262178 | NEK9 | 0.53 |
| 14 | 75778 | 2264415 | GSTZ1 | 0.48 |
| 14 | 75883 | 2264520 | C14orf133 | 0.54 |
| 14 | 76129 | 2264767 | ALKBH | 0.46 |
| 14 | 88853 | 2277491 | CALM1 | 0.53 |
| 14 | 91396 | 22280034 | ITPK1 | 0.51 |
| 14 | 92507 | 2281145 | DDX24 | 0.46 |
| 14 | 94000 | 2282638 | C14orf87 | 0.51 |
| 14 | 97854 | 2286492 | C14orf154 | 0.57 |
| 14 | 98838 | 2287476 | MGC4645 | 0.52 |
| 14 | 101389 | 2290026 | CDC42BPB | 0.51 |
| 14 | 102013 | 2290651 | BAG5 | 0.59 |
| 14 | 102369 | 2291006 | C14orf2 | 0.44 |
| 14 | 103207 | 2291845 | AKT1 | 0.46 |
| 14 | 103314 | 2291952 | KIAA02284 | 0.46 |
| 14 | 103830 | 2292467 | PACS2 | 0.53 |
| 15 | 38776 | 2332725 | FLJ10634 | 0.48 |
| 15 | 40167 | 2334116 | VPS39 | 0.49 |
| 15 | 59681 | 2353809 | VPS13C | 0.46 |
| 15 | 62396 | 2356344 | TRIP4 | 0.45 |
| 15 | 62682 | 2356631 | ZNF609 | 0.45 |
| 15 | 63266 | 2357215 | PARP16 | 0.48 |
| 15 | 63455 | 2357403 | DPP8 | 0.48 |
| 15 | 63587 | 2357535 | DKFZP564O1664 | 0.55 |
| 15 | 63878 | 2357826 | RAB11A | 0.57 |
| 15 | 64503 | 2358451 | SNAPC5 | 0.53 |
| 15 | 64507 | 2358456 | RPL4 | 0.55 |
| 15 | 64513 | 2358462 | FLJ10036 | 0.51 |
| 15 | 66062 | 2360011 | PIAS1 | 0.47 |
| 15 | 70482 | 2364431 | ARIH1 | 0.48 |
| 15 | 72617 | 2366565 | CLK3 | 0.48 |
| 15 | 73344 | 2367293 | COMMD4 | 0.47 |
| 15 | 73475 | 2367424 | PTPN9 | 0.48 |
| 15 | 73647 | 2367596 | C15orf12 | 0.48 |
| 15 | 76304 | 2370252 | REC14 | 0.65 |
| 15 | 86732 | 2380681 | MRPL46 | 0.46 |
| 15 | 86740 | 2380689 | MRPS11 | 0.50 |
| 15 | 86740 | 2380689 | MRPS11 | 0.50 |
| 15 | 97991 | 2391940 | MEF2A | 0.58 |
| 15 | 99557 | 2393506 | SNRPA1 | 0.54 |
| 15 | 99557 | SNRPA1 | 0.54 | |
| 15 | 99917 | 2393866 | BLP2 | 0.59 |
| 16 | 37 | 2394242 | POLR3K | 0.48 |
| 16 | 48 | 2394253 | RHBDF1 | 0.56 |
| 16 | 69 | 2394274 | MPG | 0.64 |
| 16 | 387 | 2394592 | NME4 | 0.48 |
| 16 | 392 | 2394597 | DECR2 | 0.61 |
| 16 | 560 | 2394765 | PIGQ | 0.48 |
| 16 | 658 | 2394863 | RHIT2 | 0.58 |
| 16 | 670 | 2394876 | STUB1 | 0.54 |
| 16 | 711 | 2394916 | MGC2494 | 0.57 |
| 16 | 720 | 2394925 | NARFL | 0.47 |
| 16 | 844 | 2395049 | FLJ12681 | 0.51 |
| 16 | 1483 | 2395689 | KIAA0683 | 0.55 |
| 16 | 1500 | 2395706 | KIAA0590 | 0.60 |
| 16 | 1668 | 2395873 | C16orf34 | 0.51 |
| 16 | 1760 | 2395966 | NME3 | 0.49 |
| 16 | 1974 | 2396179 | GFER | 0.50 |
| 16 | 2214 | 2396419 | E4F1 | 0.48 |
| 16 | 2528 | 2396733 | PDPK1 | 0.58 |
| 16 | 2822 | 2397027 | TCEB2 | 0.49 |
| 16 | 3073 | 2397278 | HCFC1R1 | 0.58 |
| 16 | 3179 | 2397384 | LOC440334 | 0.49 |
| 16 | 3334 | 2397539 | ZNF263 | 0.58 |
| 16 | 3432 | 2397638 | ZNF434 | 0.56 |
| 16 | 3452 | 2397657 | ZNF174 | 0.54 |
| 16 | 3508 | 2397714 | FLJ14154 | 0.55 |
| 16 | 3560 | 2397765 | CLUAP1 | 0.53 |
| 16 | 3706 | 2397914 | TRAP1 | 0.54 |
| 16 | 3777 | 2397982 | CREBBP | 0.56 |
| 16 | 3777 | 2397982 | CREBBP | 0.56 |
| 16 | 4015 | 23962220 | ADCY9 | 0.45 |
| 16 | 4391 | 2398596 | Magmas | 0.48 |
| 16 | 30698 | 2424903 | MGC3121 | 0.46 |
| 16 | 30713 | 2424918 | FBS1 | 0.48 |
| 16 | 31081 | 2425286 | STX4A | 0.48 |
| 16 | 31156 | 2425361 | BCKDK | 0.54 |
| 16 | 31165 | 2425370 | MYST1 | 0.47 |
| 16 | 31179 | 2425384 | PRSS8 | 0.45 |
| 16 | 31537 | 2425742 | FLJ13868 | 0.64 |
| 16 | 46475 | 2440680 | BPS35 | 0.49 |
| 16 | 47276 | 2441482 | PHK8 | 0.48 |
| 16 | 48172 | 2442378 | SIAH1 | 0.54 |
| 16 | 48354 | 2442560 | N4BP1 | 0.54 |
| 16 | 53044 | 2447249 | CHD9 | 0.50 |
| 16 | 53247 | 2447452 | RBL2 | 0.48 |
| 16 | 57272 | 2451477 | DOK4 | 0.59 |
| 16 | 57272 | 2451477 | POLR2C | 0.49 |
| 16 | 57547 | 2451752 | KATNB1 | 0.56 |
| 16 | 57811 | 2452016 | FLJ13154 | 0.46 |
| 16 | 57923 | 2452128 | GTL3 | 0.50 |
| 16 | 57967 | 2452172 | CSNK2A2 | 0.52 |
| 16 | 58325 | 2452530 | FLJ21148 | 0.54 |
| 16 | 58330 | 2452535 | CNOT1 | 0.62 |
| 16 | 58516 | 2452722 | GOT2 | 0.46 |
| 16 | 66534 | 2460739 | DNCLI2 | 0.61 |
| 16 | 66742 | 2460947 | CGI-128 | 0.54 |
| 16 | 66840 | 2461045 | CBFB | 0.49 |
| 16 | 66964 | 2461170 | TRADD | 0.51 |
| 16 | 67037 | 2461242 | HSPC171 | 0.50 |
| 16 | 67248 | 2461453 | ATP6V0D1 | 0.45 |
| 16 | 67468 | 2461673 | ACD | 0.51 |
| 16 | 67485 | 2461690 | MGC11335 | 0.45 |
| 16 | 67533 | 2461738 | RANBP10 | 0.56 |
| 16 | 67652 | 22461858 | THAP11 | 0.48 |
| 16 | 67657 | 2461862 | NUTF2 | 0.57 |
| 16 | 67831 | 2462037 | DDX28 | 0.55 |
| 16 | 67896 | 2462101 | NFATC3 | 0.49 |
| 16 | 68077 | 2462282 | SLC7A6 | 0.49 |
| 16 | 69121 | 2463327 | VPS4A | 0.49 |
| 16 | 69572 | 2463778 | WWP2 | 0.47 |
| 16 | 70063 | 2464268 | AARS | 0.52 |
| 16 | 70157 | 2464362 | DDX19L | 0.57 |
| 16 | 70334 | 2464539 | SFB3B3 | 0.54 |
| 16 | 70498 | 2464703 | VAC14 | 0.54 |
| 16 | 71541 | 2465746 | AP1G1 | 0.53 |
| 16 | 71706 | 2465911 | KIAA0174 | 0.59 |
| 16 | 71904 | 2466109 | DHX38 | 0.66 |
| 16 | 74110 | 2468315 | PSMD7 | 0.51 |
| 16 | 74266 | 2468471 | GLG1 | 0.50 |
| 16 | 74435 | 2468640 | RFWD3 | 0.53 |
| 16 | 75107 | 2469312 | CFDP1 | 0.48 |
| 16 | 75441 | 2469646 | KARS | 0.64 |
| 16 | 75461 | 2469666 | TERF2IP | 0.53 |
| 16 | 77005 | 2471210 | MON1B | 0.62 |
| 16 | 80789 | 2474994 | DC13 | 0.54 |
| 16 | 80854 | 2475059 | KIAA0431 | 0.51 |
| 16 | 83621 | 2477827 | HSBP1 | 0.53 |
| 16 | 83712 | 2477918 | MLYCD | 0.47 |
| 16 | 83867 | 2478072 | MBTPS1 | 0.46 |
| 16 | 82991 | 2478196 | TAF1C | 0.446 |
| 16 | 84293 | 2478496 | KIAA1609 | 0.48 |
| 16 | 84462 | 2478667 | C16orf44 | 0.46 |
| 16 | 84513 | 2478718 | USP10 | 0.70 |
| 16 | 84788 | 2478993 | ZDHHC7 | 0.50 |
| 16 | 85594 | 2479799 | NOC4 | 0.56 |
| 16 | 85615 | 2479820 | COX411 | 0.66 |
| 16 | 85346 | 2480551 | FLJ12998 | 0.51 |
| 16 | 87214 | 2481419 | MAP1LC3B | 0.49 |
| 16 | 88620 | 2482825 | APRT | 0.48 |
| 16 | 88668 | 2482873 | HSPC176 | 0.54 |
| 16 | 89095 | 2483300 | ANKRD11 | 0.54 |
| 16 | 89371 | 2483576 | LOC388344 | 0.55 |
| 16 | 89684 | 2483889 | KIAA1049 | 0.54 |
| 16 | 89757 | 2483963 | FLJ20186 | 0.54 |
| 17 | 670 | 2484917 | RNMTL1 | 0.47 |
| 17 | 1761 | 2486008 | PRPF8 | 0.56 |
| 17 | 2140 | 2486387 | DPH2L1 | 0.48 |
| 17 | 2433 | 2486680 | FLJ10534 | 0.48 |
| 17 | 2433 | 2486680 | SRR | 0.58 |
| 17 | 2704 | 2486951 | PAFAH1B1 | 0.52 |
| 17 | 7324 | 2491571 | ACADVL | 0.58 |
| 17 | 7348 | 2491595 | DULLARD | 0.46 |
| 17 | 7417 | 2491664 | GPS2 | 0.46 |
| 17 | 7494 | 2491741 | PLSCR3 | 0.47 |
| 17 | 16049 | 2500296 | ADORA2B | 0.48 |
| 17 | 16103 | TTC19 | 0.46 | |
| 17 | 17269 | 2501516 | M-RIP | 0.52 |
| 17 | 18349 | 2502596 | FLII | 0.54 |
| 17 | 18962 | 2503209 | PRPSAP2 | 0.45 |
| 17 | 19303 | 2503550 | EPN2 | 0.49 |
| 17 | 19444 | 2503691 | MAPK7 | 0.55 |
| 17 | 21192 | 2505439 | DKFZp566O084 | 0.48 |
| 17 | 21263 | 2505510 | C17orf35 | 0.46 |
| 17 | 21357 | 2505604 | MAP2K3 | 0.45 |
| 17 | 28046 | 2512293 | GIT1 | 0.52 |
| 17 | 28852 | 2513099 | CPD | 0.49 |
| 17 | 28950 | 2513197 | GOSR1 | 0.60 |
| 17 | 30336 | 2514583 | HCA66 | 0.52 |
| 17 | 30410 | 2514657 | LOC440423 | 0.59 |
| 17 | 30615 | 2514862 | RHOT1 | 0.47 |
| 17 | 30804 | 2515051 | MNJMU-R1 | 0.61 |
| 17 | 30917 | 2515164 | PSMD11 | 0.47 |
| 17 | 30960 | 2515207 | CDK5R1 | 0.65 |
| 17 | 30960 | 2515207 | CDK5R1 | 0.65 |
| 17 | 32743 | 2516990 | CCL7 | 0.61 |
| 17 | 33400 | 2517648 | CCT6B | 0.47 |
| 17 | 33453 | 2517700 | LIG3 | 0.63 |
| 17 | 33573 | 2517820 | RAD51L3 | 0.71 |
| 17 | 33604 | 2517851 | FLJ10458 | 0.67 |
| 17 | 38168 | 2522415 | STARD3 | 0.86 |
| 17 | 28197 | 2522444 | TCAP | 0.55 |
| 17 | 38199 | 2522447 | PNMT | 0.54 |
| 17 | 38202 | 2522449 | PERLD1 | 0.88 |
| 17 | 38231 | 2522478 | ERBB2 | 0.86 |
| 17 | 38269 | 2522516 | GRB7 | 0.84 |
| 17 | 38448 | 2522695 | GSDML | 0.66 |
| 17 | 38512 | 2522759 | PSMD3 | 0.73 |
| 17 | 38550 | 2522797 | THRAP4 | 0.77 |
| 17 | 38550 | 2522797 | THRAP4 | 0.77 |
| 17 | 38672 | 2522919 | CASC3 | 0.58 |
| 17 | 38792 | 25223039 | WIRE | 0.57 |
| 17 | 39158 | 2523405 | SMARCE1 | 0.57 |
| 17 | 39158 | 2523405 | SMARCE1 | 0.57 |
| 17 | 39348 | 2523595 | KRT10 | 0.70 |
| 17 | 41087 | 2525334 | COASY | 0.49 |
| 17 | 41092 | 2525339 | MLX | 0.53 |
| 17 | 41225 | 2525473 | EZH1 | 0.50 |
| 17 | 41359 | 2525606 | PSME3 | 0.51 |
| 17 | 41476 | 2525723 | MGC2744 | 0.49 |
| 17 | 41551 | 2525798 | RND2 | 0.44 |
| 17 | 41696 | 2525943 | MBR1 | 0.48 |
| 17 | 42036 | 2526284 | DHX4 | 0.48 |
| 17 | 43614 | 2527861 | NMT1 | 0.57 |
| 17 | 43990 | 2528237 | PLEKHM1 | 0.52 |
| 17 | 44067 | 2528315 | LOC9884 | 0.49 |
| 17 | 46494 | 2530471 | PNPO | 0.44 |
| 17 | 47445 | 2531692 | ATP5G1 | 0.60 |
| 17 | 47461 | 2531706 | FLJ13855 | 0.69 |
| 17 | 47482 | 2531729 | EAP30 | 0.78 |
| 17 | 47847 | 2532094 | ZNF652 | 0.50 |
| 17 | 47956 | 2532203 | PHB | 0.64 |
| 17 | 48152 | 2532399 | SPCP | 0.55 |
| 17 | 48253 | 2532500 | SLC35B1 | 0.79 |
| 17 | 48341 | 2532588 | MYST2 | 0.78 |
| 17 | 48525 | 2532772 | DLX4 | 0.67 |
| 17 | 48808 | 2532855 | ITGA3 | 0.58 |
| 17 | 48647 | 2532894 | PDK2 | 0.49 |
| 17 | 48896 | 2533145 | XYLT2 | 0.74 |
| 17 | 48933 | 2533180 | PRO1855 | 0.50 |
| 17 | 48978 | 2533225 | FLJ20920 | 0.45 |
| 17 | 49031 | 2533278 | RSAD1 | 0.63 |
| 17 | 49031 | 2533332 | EPN3 | 0.55 |
| 17 | 49099 | 2533346 | SSP411 | 0.57 |
| 17 | 49247 | 2533494 | MGC15396 | 0.46 |
| 17 | 49272 | 2533519 | CROP | 0.68 |
| 17 | 49414 | 2533661 | TOB1 | 0.54 |
| 17 | 49518 | 2533765 | SPAG9 | 0.59 |
| 17 | 49706 | 2533953 | NME1 | 0.59 |
| 17 | 49718 | 2533966 | NME2 | 0.72 |
| 17 | 55386 | 2539633 | DGKE | 0.65 |
| 17 | 55490 | 2539737 | COIL | 0.86 |
| 17 | 55637 | 2539884 | AKAP1 | 0.69 |
| 17 | 56757 | 2541055 | FLJ20345 | 0.48 |
| 17 | 56897 | 2541144 | SUPT4H1 | 0.58 |
| 17 | 56906 | 2541153 | FLJ20315 | 0.64 |
| 17 | 57042 | 2541289 | MTMR4 | 0.47 |
| 17 | 57109 | 2541356 | TEX14 | 0.49 |
| 17 | 57245 | 2541492 | RAD51C | 0.77 |
| 17 | 57550 | 2541797 | TRIM37 | 0.66 |
| 17 | 57762 | 2542009 | FLJ10587 | 0.73 |
| 17 | 58117 | 2542364 | DHX40 | 0.71 |
| 17 | 58172 | 2542419 | CLTC | 0.70 |
| 17 | 58249 | 2542496 | BIT1 | 0.78 |
| 17 | 58249 | 2542496 | BIT1 | 0.78 |
| 17 | 58259 | 2542507 | VMP1 | 0.52 |
| 17 | 58412 | 2542659 | TUBD1 | 0.65 |
| 17 | 58445 | 2542692 | RPS6KB1 | 0.69 |
| 17 | 58445 | 2542692 | RPS6KB1 | 0.69 |
| 17 | 58504 | 2542751 | LOC51136 | 0.52 |
| 17 | 58595 | 2542842 | ABC1 | 0.79 |
| 17 | 58731 | 2542978 | USP32 | 0.70 |
| 17 | 58995 | 2543242 | APPBP2 | 0.78 |
| 17 | 59152 | 2543399 | PPM1D | 0.59 |
| 17 | 59230 | 2543477 | BCAS3 | 0.61 |
| 17 | 60234 | 2544482 | BCAS3 | 0.61 |
| 17 | 60234 | 2544482 | BRIP1 | 0.50 |
| 17 | 60497 | 2544745 | THRAP1 | 0.70 |
| 17 | 61030 | 2542577 | TLK2 | 0.77 |
| 17 | 61940 | 2546187 | DKFZP564D166 | 0.59 |
| 17 | 61984 | 2546231 | CYB561 | 0.61 |
| 17 | 62142 | 2546389 | HAN11 | 0.49 |
| 17 | 62254 | 2546501 | LYK5 | 0.56 |
| 17 | 62343 | 2546590 | DDX42 | 0.52 |
| 17 | 62370 | 2546617 | FTSJ3 | 0.52 |
| 17 | 62383 | 2546630 | SMARCD2 | 0.48 |
| 17 | 65578 | 2549826 | CACNG4 | 0.64 |
| 17 | 65624 | 2549871 | HELZ | 0.63 |
| 17 | 53884 | 2550131 | PSMD12 | 0.55 |
| 17 | 65924 | 2550171 | PITPNC1 | 0.50 |
| 17 | 66264 | 2550511 | DKFZP586L0724 | 0.51 |
| 17 | 71763 | 2556010 | SSTR2 | 0.65 |
| 17 | 71877 | 2556124 | CDC42EP4 | 0.52 |
| 17 | 73033 | 2557280 | GPRC5C | 0.48 |
| 17 | 73364 | 2557611 | EBSP | 0.63 |
| 17 | 73370 | 2557617 | FLJ20255 | 0.62 |
| 17 | 73456 | 2557703 | FDXR | 0.45 |
| 17 | 73581 | 2557828 | HUMPPA | 0.65 |
| 17 | 73606 | 2557853 | ICT1 | 0.73 |
| 17 | 73632 | 2557879 | ATP5H | 0.59 |
| 17 | 73640 | 2557888 | KCTD2 | 0.66 |
| 17 | 73681 | 2557928 | SLC16A5 | 0.59 |
| 17 | 73703 | 2557950 | ARMC7 | 0.64 |
| 17 | 73729 | 2557976 | HN1 | 0.50 |
| 17 | 73761 | 2558008 | SUMO2 | 0.59 |
| 17 | 73799 | 2558046 | PCNT1 | 0.57 |
| 17 | 73830 | 2558077 | GGA3 | 0.64 |
| 17 | 73855 | 2558102 | MRPS7 | 0.59 |
| 17 | 73911 | 2558158 | GRB2 | 0.60 |
| 17 | 74050 | 2558297 | KIAA0195 | 0.66 |
| 17 | 74093 | 2558341 | CASKIN2 | 0.66 |
| 17 | 744093 | 2558341 | CASKIN2 | 0.66 |
| 17 | 74119 | 2558366 | LLGL2 | 0.54 |
| 17 | 74220 | 2558467 | RECQL5 | 0.64 |
| 17 | 74261 | 2558508 | HCNGP | 0.56 |
| 17 | 74439 | 2558686 | WBP2 | 0.49 |
| 17 | 74600 | 2558847 | EVPL | 0.57 |
| 17 | 74677 | 2558924 | EXOC7 | 0.59 |
| 17 | 74983 | 2559230 | E2-230K | 0.71 |
| 17 | 75064 | 2559311 | RHBDL6 | 0.47 |
| 17 | 75913 | 2560160 | 9-Sep | 0.58 |
| 17 | 76706 | 2560953 | EVER1 | 0.54 |
| 17 | 76762 | 2561009 | SYNGR2 | 0.51 |
| 17 | 76807 | 2561055 | BIRC5 | 0.48 |
| 17 | 76972 | 2561219 | PGS1 | 0.66 |
| 17 | 77267 | 2561514 | PSCD1 | 0.49 |
| 17 | 79709 | 2563956 | BAIAP2 | 0.49 |
| 17 | 79864 | 2564111 | AZI1 | 0.44 |
| 17 | 81095 | 2565343 | NARF | 0.49 |
| 17 | 81156 | 2565404 | FOXK2 | 0.50 |
| 17 | 81251 | 2565498 | WDR45L | 0.55 |
| 17 | 81294 | 2565541 | RAB40B | 0.56 |
| 17 | 81353 | 2565601 | FM3KRP | 0.53 |
| 18 | 149 | 2566256 | USP14 | 0.48 |
| 18 | 205 | 22566312 | THOC1 | 0.52 |
| 18 | 712 | 2566819 | YES1 | 0.57 |
| 18 | 2528 | 2568636 | METTL4 | 0.57 |
| 18 | 2562 | 2568669 | KNTC2 | 0.45 |
| 18 | 2793 | 2568900 | SMCHD1 | 0.57 |
| 18 | 3252 | 2569360 | MRLC2 | 0.52 |
| 18 | 9093 | 2575200 | NDUFV2 | 0.51 |
| 18 | 9172 | 2575280 | ANKRD12 | 0.57 |
| 18 | 9466 | 2575573 | RALBP1 | 0.64 |
| 18 | 9537 | 2575644 | PP4R1 | 0.53 |
| 18 | 10516 | 2576623 | NAPG | 0.47 |
| 18 | 11841 | 2577949 | CHMP1B | 0.62 |
| 18 | 11874 | 2577981 | MPPE1 | 0.57 |
| 18 | 11971 | 2578079 | IMPA2 | 0.60 |
| 18 | 12298 | 2578406 | TUBB6 | 0.51 |
| 18 | 12319 | 2578426 | AFG3L2 | 0.73 |
| 18 | 12422 | 2578529 | C18ord43 | 0.45 |
| 18 | 12663 | 2578770 | C18orf9 | 0.55 |
| 18 | 12693 | 2578800 | TNFSG5IP1 | 0.63 |
| 18 | 12783 | 2578891 | PTPN1 | 0.57 |
| 18 | 12938 | 2579045 | SEH1L | 0.69 |
| 18 | 13717 | 2579824 | RNMT | 0.68 |
| 18 | 19335 | 2585443 | C18orf8 | 0.51 |
| 18 | 19363 | 2585471 | NPC1 | 0.52 |
| 18 | 20259 | 2586366 | IMPACT | 0.47 |
| 18 | 27662 | 2593770 | KIAA1012 | 0.53 |
| 18 | 31122 | 2597230 | ZNF271 | 0.45 |
| 18 | 32626 | 2598735 | C18orf10 | 0.54 |
| 18 | 37787 | 2603895 | PIK3C3 | 0.48 |
| 18 | 43620 | 2609727 | SMAD2 | 0.59 |
| 18 | 45267 | 2611374 | RPL17 | 0.46 |
| 18 | 46047 | 2612155 | MBD1 | 0.58 |
| 18 | 46061 | 2612168 | CXXC1 | 0.55 |
| 18 | 57860 | 2623968 | PIGN | 0.50 |
| 18 | 58391 | 2624498 | ZCCHC2 | 0.51 |
| 18 | 585333 | 2624640 | PHLPP | 0.57 |
| 18 | 59147 | 2625254 | FVT1 | 0.76 |
| 18 | 59205 | 2625313 | VPS48 | 0.49 |
| 18 | 75761 | 2641869 | PQLC1 | 0.63 |
| 18 | 75832 | 2641939 | TXNL4A | 0.63 |
| 18 | 75893 | 2642001 | C18orf22 | 0.60 |
| 18 | 75966 | 2642074 | KIAA0863 | 0.49 |
| 19 | 12673 | 2654896 | TNPO2 | 0.46 |
| 19 | 15325 | 2657548 | AKAP8 | 0.57 |
| 19 | 15394 | 2657616 | WIZ | 0.52 |
| 19 | 17309 | 2659352 | GTPBP3 | 0.59 |
| 19 | 17483 | 2659706 | PGLS | 0.50 |
| 19 | 18804 | 2661026 | RENT1 | 0.48 |
| 19 | 18891 | 2861114 | HOMER3 | 0.54 |
| 19 | 18892 | 2661114 | DDX49 | 0.57 |
| 19 | 19091 | 2661314 | FLJ20422 | 0.54 |
| 19 | 19317 | 2661540 | KIAA0892 | 0.47 |
| 19 | 34390 | 2676613 | UQCRFS1 | 0.54 |
| 19 | 34789 | 2677012 | POP4 | 0.67 |
| 19 | 34848 | 2677070 | PLENKF1 | 0.55 |
| 19 | 34995 | 2677217 | CCNE1 | 0.67 |
| 19 | 35125 | 2677348 | C19orf2 | 0.73 |
| 19 | 40812 | 2683034 | MGC10433 | 0.46 |
| 19 | 42815 | 2685038 | KIAA0961 | 0.48 |
| 19 | 43802 | 2666024 | EIF2S12 | 0.57 |
| 19 | 43830 | 2686053 | ACTN4 | 0.47 |
| 19 | 48792 | 2691015 | ANF576 | 0.45 |
| 19 | 50575 | 2692797 | PPP1R13L | 0.46 |
| 19 | 50605 | 2692827 | ERCC1 | 0.54 |
| 19 | 54309 | 2696532 | LIN7B | 0.49 |
| 19 | 54641 | 2696864 | FLJ20643 | 0.78 |
| 19 | 54683 | 2696905 | RPL13A | 0.49 |
| 19 | 54708 | 2696931 | FCGRT | 0.52 |
| 19 | 54751 | 2696973 | NOSIP | 0.71 |
| 19 | 54778 | 2697001 | PRRG2 | 0.53 |
| 19 | 54855 | 2697077 | IRF3 | 0.54 |
| 19 | 55013 | 2697236 | MED25 | 0.45 |
| 19 | 55046 | 2697269 | PTOV1 | 0.47 |
| 19 | 55056 | 2697279 | PNKP | 0.51 |
| 19 | 55073 | 26972295 | TBC1D17 | 0.44 |
| 19 | 55102 | 2697324 | NUP62 | 0.57 |
| 19 | 55172 | 2697394 | VRK3 | 0.56 |
| 19 | 55221 | 2697444 | ZNF473 | 0.52 |
| 19 | 60433 | 2702655 | KIAA1115 | 0.51 |
| 19 | 60465 | 2702668 | HSPBP1 | 0.49 |
| 19 | 60656 | 2702879 | ISOC2 | 0.54 |
| 19 | 60845 | 2703067 | ZNF580 | 0.45 |
| 19 | 62554 | 2704777 | ZNF304 | 0.50 |
| 19 | 622691 | 2704913 | ZNF419 | 0.61 |
| 19 | 622817 | 2705040 | ZNF134 | 0.50 |
| 19 | 62885 | 2705108 | ZNF551 | 0.52 |
| 19 | 63386 | 2705609 | ZNF274 | 0.44 |
| 19 | 63482 | 2705705 | ZNF8 | 0.47 |
| 19 | 63661 | 2705883 | FLJ45850 | 0.49 |
| 19 | 63670 | 2705893 | ZNF324 | 0.65 |
| 19 | 63748 | 2705970 | TRIM28 | 0.49 |
| 19 | 63759 | 2705981 | UBE2M | 0.44 |
| 20 | 459 | 2706493 | CSNK2A1 | 0.48 |
| 20 | 1418 | 2707452 | NSFL1C | 0.45 |
| 20 | 3185 | 2709219 | ITPA | 0.48 |
| 20 | 20010 | 2726044 | CRNKL1 | 0.444 |
| 20 | 32662 | 2738696 | CDKSRAP1 | 0.50 |
| 20 | 34018 | 2740052 | NCOA6 | 0.73 |
| 20 | 34232 | 2740266 | GSS | 0.69 |
| 20 | 34306 | 2740340 | TRPC4AP | 0.73 |
| 20 | 34419 | 2740453 | C2orf31 | 0.49 |
| 20 | 34530 | 2740564 | LOC400843 | 0.65 |
| 20 | 34582 | 2740616 | ITGB4BP | 0.67 |
| 20 | 34606 | 2740640 | C20orf44 | 0.59 |
| 20 | 34759 | 2740793 | CEP2 | 0.72 |
| 20 | 34919 | 2740953 | SPAG4 | 0.48 |
| 20 | 34929 | 2740964 | CPNE1 | 0.66 |
| 20 | 34998 | 2741032 | NFS1 | 0.71 |
| 20 | 35007 | 2741041 | PRNC2 | 0.60 |
| 20 | 35105 | 2741139 | PHF20 | 0.68 |
| 20 | 35257 | 2741291 | SCAND1 | 0.73 |
| 20 | 35458 | 2741492 | EPB41L1 | 0.68 |
| 20 | 35540 | 2741574 | C20orf4 | 0.79 |
| 20 | 35681 | 2741715 | DLGAP4 | 0.67 |
| 20 | 35920 | 2741954 | C20orf24 | 0.54 |
| 20 | 35966 | 2742000 | NDRG3 | 0.68 |
| 20 | 36066 | 2742100 | C20orf172 | 0.48 |
| 20 | 36105 | 2742139 | KIAA0889 | 0.70 |
| 20 | 36493 | 2742527 | RPN2 | 0.67 |
| 20 | 38063 | 2744097 | ACTR5 | 0.68 |
| 20 | 38276 | 2744311 | DHX35 | 0.70 |
| 20 | 40419 | 2746453 | ZHX3 | 0.51 |
| 20 | 40452 | 2746486 | PLCG1 | 0.45 |
| 20 | 43511 | 2749545 | C20orf111 | 0.69 |
| 20 | 43814 | 2749848 | C20orf121 | 0.69 |
| 20 | 43814 | 2749848 | TDE1 | 0.54 |
| 20 | 43846 | 2749880 | PKIG | 0.54 |
| 20 | 44200 | 2750234 | YWHAB | 0.51 |
| 20 | 44256 | 2750290 | TOMM34 | 0.58 |
| 20 | 45156 | 2751190 | PTE1 | 0.52 |
| 20 | 45206 | 2751240 | PPGB | 0.45 |
| 20 | 45228 | 2751262 | SLC12A5 | 0.47 |
| 20 | 46816 | 2752850 | NCOA3 | 0.54 |
| 20 | 48224 | 2754258 | ARGEF2 | 0.65 |
| 20 | 48348 | 2754382 | CSE1L | 0.67 |
| 20 | 48415 | 2754450 | STAU | 0.69 |
| 20 | 48521 | 2754555 | DDX27 | 0.78 |
| 20 | 48935 | 2754969 | B4GALT5 | 0.48 |
| 20 | 49148 | 2755183 | SLC9A8 | 0.73 |
| 20 | 49205 | 2755240 | SPATA2 | 0.77 |
| 20 | 49238 | 2755273 | ZNF313 | 0.46 |
| 20 | 49383 | 2755417 | UBE2V1 | 0.76 |
| 20 | 49812 | 2755846 | PTPN1 | 0.49 |
| 20 | 50020 | 2756054 | PARD6B | 0.52 |
| 20 | 50237 | 2756271 | DPM1 | 0.49 |
| 20 | 50261 | 2756295 | MOCS3 | 0.56 |
| 20 | 50899 | 2756933 | ATP9A | 0.52 |
| 20 | 51453 | 2757487 | ZFP64 | 0.53 |
| 20 | 52869 | 2758903 | ZNF217 | 0.50 |
| 20 | 53510 | 2759544 | PFDN4 | 0.71 |
| 20 | 55653 | 2761687 | CSTF1 | 0.56 |
| 20 | 55729 | 2761763 | C20orf43 | 0.67 |
| 20 | 55890 | 2761924 | TFAP2C | 0.51 |
| 20 | 56612 | 2762646 | RAE1 | 0.76 |
| 20 | 56619 | 2762654 | RNPC1 | 0.45 |
| 20 | 56822 | 2762856 | PCK1 | 0.58 |
| 20 | 57627 | 2763662 | RAB22A | 0.72 |
| 20 | 57650 | 2763684 | VAPB | 0.75 |
| 20 | 57912 | 2753947 | STX16 | 0.82 |
| 20 | 57953 | 2763987 | NPEPL1 | 0.69 |
| 20 | 63054 | 2769098 | ARFRP1 | 0.60 |
| 20 | 63093 | 2769127 | ZGPAT | 0.58 |
| 20 | 63098 | 2769132 | SLC2A4RG | 0.50 |
| 20 | 63223 | 2769257 | TPD52L2 | 0.57 |
| 20 | 63223 | 2769257 | TPD52L2 | 0.57 |
| 20 | 63298 | 2769332 | UCKL1 | 0.53 |
| 20 | 63339 | 2769373 | C20orf14 | 0.50 |
| 20 | 63431 | 2769465 | TGS19 | 0.45 |
| 21 | 26030 | 2795806 | GABPA | 0.48 |
| 21 | 33836 | 2803612 | SON | 0.46 |
| 21 | 36665 | 2806441 | ZCWCC3 | 0.48 |
| 21 | 36679 | 2806455 | CHAF1B | 0.50 |
| 21 | 44383 | 2814160 | PWP2H | 0.51 |
| 21 | 44410 | 2814186 | C21orf33 | 0.44 |
| 21 | 45045 | 2814821 | UBE2G2 | 0.52 |
| 21 | 45082 | 2814858 | SUMO3 | 0.49 |
| 21 | 45126 | 2814902 | PTTG1IP | 0.47 |
| 21 | 46600 | 2816376 | PCNT2 | 0.56 |
| 21 | 46912 | 2816688 | HRMT1L1 | 0.53 |
| 22 | 15993 | 2832745 | CECR5 | 0.48 |
| 22 | 16546 | 2833298 | BCL2L13 | 0.48 |
| 22 | 16546 | 2833298 | BCL2L13 | 0.48 |
| 22 | 16645 | 2833397 | MICAL3 | 0.47 |
| 22 | 16935 | 2833687 | PEX26 | 0.50 |
| 22 | 17496 | 2834248 | DGCR14 | 0.50 |
| 22 | 17693 | 2834445 | HIRA | 0.45 |
| 22 | 17813 | 2834565 | UFD1L | 0.54 |
| 22 | 18304 | 2835056 | COMT | 0.55 |
| 23 | 18480 | 2835232 | RANBP1 | 0.58 |
| 22 | 19120 | 2835873 | KELCHL | 0.47 |
| 22 | 19538 | 2836290 | SNAP29 | 0.45 |
| 22 | 26492 | 2842344 | MN1 | 0.70 |
| 22 | 27408 | 2844160 | CHEK2 | 0.52 |
| 22 | 28048 | 2844800 | AP1B1 | 0.48 |
| 22 | 28226 | 2844979 | C22ord19 | 0.66 |
| 22 | 28275 | 2845027 | NIPSNAP1 | 0.48 |
| 22 | 28488 | 2845240 | UCRC | 0.47 |
| 22 | 2927 | 2846049 | PES1 | 0.64 |
| 22 | 29566 | 2846318 | ZCWCC1 | 0.49 |
| 22 | 26692 | 2846444 | FLJ20618 | 0.51 |
| 22 | 29969 | 2846721 | LIMK2 | 0.61 |
| 22 | 30120 | 2846872 | DRG1 | 0.68 |
| 22 | 30475 | 2847228 | DEPDC5 | 0.46 |
| 22 | 31108 | 2847860 | HSPC117 | 0.56 |
| 22 | 31195 | 2847947 | FBXO7 | 0.54 |
| 22 | 35087 | 2851839 | FLJ23322 | 0.50 |
| 22 | 35106 | 2851858 | TXN2 | 0.45 |
| 22 | 35150 | 2851902 | EIF3S7 | 0.54 |
| 22 | 36200 | 2852952 | CDC42EP1 | 0.50 |
| 22 | 36488 | 2853241 | EIF3S6IP | 0.53 |
| 22 | 36488 | 2853241 | EIF3S6IP | 0.53 |
| 22 | 36551 | 2853304 | MICAL-1 | 0.52 |
| 22 | 36593 | 2853345 | POLR2F | 0.47 |
| 22 | 37267 | 2854019 | GTPBP1 | 0.557 |
| 22 | 37325 | 2854077 | KIAA0063 | 0.52 |
| 22 | 37621 | 2854374 | APOBEC3B | 0.49 |
| 22 | 38003 | 2854755 | SYNGR1 | 0.52 |
| 22 | 38141 | 2854894 | FLJ20232 | 0.55 |
| 22 | 38962 | 2855714 | TNRC6B | 0.45 |
| 22 | 338966 | 2855738 | ADSL | 0.49 |
| 22 | 29464 | 2856216 | ST13 | 0.53 |
| 22 | 39590 | 2856343 | RBX1 | 0.45 |
| 22 | 40108 | 2856860 | ACO2 | 0.46 |
| 22 | 40136 | 2856888 | D15Wsu75e | 0.60 |
| 22 | 40260 | 2857013 | G22P1 | 0.56 |
| 22 | 40313 | 2857065 | NHP2L1 | 0.61 |
| 22 | 40472 | 2857224 | SREBF2 | 0.51 |
| 22 | 40725 | 2857477 | NDUFA6 | 0.46 |
| 22 | 40767 | 2857519 | CYP2D6 | 0.51 |
| 22 | 40787 | 2857539 | TCF20 | 0.53 |
| 22 | 41166 | 2857918 | DIA1 | 0.60 |
| 22 | 41497 | 2858249 | PACSIN2 | 0.47 |
| 22 | 41778 | 2858531 | BZRP | 0.49 |
| 22 | 42610 | 2859362 | CGI-51 | 0.49 |
| 22 | 43860 | 2860612 | NUP50 | 0.65 |
| 22 | 45293 | 2862045 | CERK | 0.48 |
| 22 | 48386 | 2865138 | BRD1 | 0.56 |
| 22 | 48467 | 2865219 | ZBED4 | 0.58 |
| 22 | 48532 | 2865284 | MGC11256 | 0.63 |
| 22 | 48806 | 2865559 | PP2447 | 0.52 |
| 22 | 48895 | 2865648 | PLXNB2 | 0.52 |
| 22 | 49002 | 2866754 | KIAA0685 | 0.58 |
| 22 | 49018 | 2865770 | SBF1 | 0.55 |
| 22 | 49071 | 2865823 | ARSA | 0.56 |
| 22 | 49074 | 2865826 | BC002942 | 0.54 |
| 22 | 49079 | 2865831 | 384D8-2 | 0.55 |
| 22 | 49094 | 2865847 | SCO2 | 0.59 |
| 22 | 49097 | 2865849 | ECGF1 | 0.47 |
| 22 | 49099 | 2865851 | LOC440836 | 0.49 |
| X | 43779 | 2909928 | UTX | 0.50 |
| X | 71910 | 2938059 | XIST | 0.67 |
| XY | 114 | 3019955 | GTPBP6 | 0.50 |
| XY | 1150 | 3020992 | SLC25A6 | 0.46 |
| XY | 1168 | 3021009 | ASMTL | 0.49 |
| XY | 2000 | 3021841 | ZBED1 | 0.45 |

TABLE 8
si RNA sequences for selected 11q13 amplicon genes

| | siRNA duplex no. | |
|---|---|---|
| Gene | sense/antisense | sequence |
| CCND1 | 1--sense | ACAACUUCCUGUCCUACUAUU (SEQ ID NO: 142) |
| CCND1 | 1--antisense | 5′-PUAGUAGGACAGGAAGUUGUUU (SEQ ID NO: 143) |
| CCND1 | 2--sense | GUUCGUGGCCUCUAAGAUGUU (SEQ ID NO: 144) |
| CCND1 | 2--antisense | 5′-PCAUCUUAGAGGCCACGAACUU (SEQ ID NO: 145) |
| CCND1 | 3--sense | GCAUGUAGUCACUUUAUAAUU (SEQ ID NO: 146) |
| CCND1 | 3--antisense | 5′-PUUAUAAAGUGACUACAUGCUU (SEQ ID NO: 147) |
| CCND1 | 4--sense | GCGUGUAGCUAUGGAAGUUUU (SEQ ID NO: 148) |
| CCND1 | 4--antisense | 5′-PAACUUCCAUAGCUACACGCUU (SEQ ID NO: 149) |
| FGF3 | 1--sense | GAGCUGGGCUAUAAUACGUUU (SEQ ID NO: 150) |
| FGF3 | 1--antisense | 5′-PACGUAUUAUAGCCCAGCUCUU (SEQ ID NO: 151) |
| FGF3 | 2--sense | GGCGGUACCUGGCCAUGAAUU (SEQ ID NO: 152) |
| FGF3 | 2--antisense | 5′-PUUCAUGGCCAGGUACCGCCUU (SEQ ID NO: 153) |
| FGF3 | 3--sense | GCGCCGAGAGACUGUGGUAUU (SEQ ID NO: 154) |
| FGF3 | 3--antisense | 5′-PUACCACAGUCUCUCGGCGCUU (SEQ ID NO: 155) |
| FGF3 | 4--sense | AGAAGCAGAGCCCGGAUAAUU (SEQ ID NO: 156) |
| FGF3 | 4--antisense | 5′-PUUAUCCGGGCUCUGCUUCUUU (SEQ ID NO: 157) |
| PPFIA1 | 1--sense | GAAGAAAGGUUACGACAGAUU (SEQ ID NO: 158) |
| PPFIA1 | 1--antisense | 5′-PUCUGUCGUAACCUUUCUUCUU (SEQ ID NO: 159) |
| PPFIA1 | 2--sense | GAGUAGCACUUGAAAGAUGUU (SEQ ID NO: 160) |
| PPFIA1 | 2--antisense | 5′-PCAUCUUUCAAGUGCUACUCUU (SEQ ID NO: 161) |
| PPFIA1 | 3--sense | AGACAACCAUAAAGUGUGAUU (SEQ ID NO: 162) |
| PPFIA1 | 3--antisense | 5′-PUCACACUUUAUGGUUGUCUUU (SEQ ID NO: 163) |
| PPFIA1 | 4--sense | AAAGGACAUUCGUGGCUUAUU (SEQ ID NO: 164) |
| PPFIA1 | 4--antisense | 5′-PUAAGCCACGAAUGUCCUUUUU (SEQ ID NO: 165) |
| FOLR3 | 1--sense | GGACGGACCUGCUCAAUGUUU (SEQ ID NO: 166) |
| FOLR3 | 1--antisense | 5′-PACAUUGAGCAGGUCCGUCCUU (SEQ ID NO: 167) |
| FOLR3 | 2--sense | GAAUUGGACCUCAGGGAUUUU (SEQ ID NO: 168) |
| FOLR3 | 2--antisense | 5′-PAAUCCCUGAGGUCCAAUUCUU (SEQ ID NO: 169) |
| FOLR3 | 3--sense | UAACUGGGAUCACUGUGGUUU (SEQ ID NO: 170) |
| FOLR3 | 3--antisense | 5′-PACCACAGUGAUCCCAGUUAUU (SEQ ID NO: 171) |
| FOLR3 | 4--sense | UCUCGUGGGAUUAUUGAUUUU (SEQ ID NO: 172) |
| FOLR3 | 4--antisense | 5′-PAAUCAAUAAUCCCACGAGAUU (SEQ ID NO: 173) |
| NEU3 | 1--sense | ACUGGAUAAUAGUGCGUAUUU (SEQ ID NO: 174) |
| NEU3 | 1--antisense | 5′-PAUACGCACUAUUAUCCAGUUU (SEQ ID NO: 175) |
| NEU3 | 2--sense | GCAGAGAAGCGUUCCACGAUU (SEQ ID NO: 176) |
| NEU3 | 2--antisense | 5′-PUCGUGGAACGCUUCUCUGCUU (SEQ ID NO: 177) |
| NEU3 | 3--sense | GAGCUGAGUUGGCGAGGGUUU (SEQ ID NO: 178) |
| NEU3 | 3--antisense | 5′-PACCCUCGCCAACUCAGCUCUU (SEQ ID NO: 179) |
| NEU3 | 4--sense | CUCAUUAGGCCCAUGGUUAUU (SEQ ID NO: 180) |
| NEU3 | 4--antisense | 5′-PUAACCAUGGGCCUAAUGAGUU (SEQ ID NO: 181) |

TABLE 9
Sequence of shRNAs targeting human NEU3, FGF3 and PPFIA1 genes

| | | |
|---|---|---|
| gene | shRNA clone | Sequence |

| | | |
|---|---|---|
| NEU3 | Hairpin sequence for TRCN0000005149 | CCGGAGTGACAACATGCTCCTTCAACTCGAGTTGAAGGAGCATGTTGTCACTTTTTT |
| | | (SEQ ID NO: 182) |
| NEU3 | Hairpin sequence for TRCN0000005148 | CCGGCCGAGCTACAGACCAATATAACTCGAGTTATATTGGTCTGTAGCTCGGTTTTT |
| | | (SEQ ID NO: 183) |
| NEU3 | Hairpin sequence for TRCN0000005147 | CCGGCCCTGCGTATACCTACTACATCTCGAGATGTAGTAGGTATACGCAGGGTTTTT |
| | | (SEQ ID NO: 184) |
| NEU3 | Hairpin sequence for TRCN0000005146 | CCGGCCTACCTTCTACAGCCTTGTACTCGAGTACAAGGCTGTAGAAGGTAGGTTTTT |
| | | (SEQ ID NO: 185) |
| NEU3 | Hairpin sequence for TRCN0000010926 | CCGGGCAGAAGAGTGGTTGTGTGTTCTCGAGAACACACAACCACTCTTCTGCTTTTT |
| | | (SEQ ID NO: 186) |
| FGF3 | Hairpin sequence for TRCN0000038162 | CCGGCAAGCTCTACTGCGCCACGAACTCGAGTTCGTGGCGCAGTAGAGCTTGTTTTTG |
| | | (SEQ ID NO: 187) |
| FGF3 | Hairpin sequence for TRCN0000038159 | CCGGACTCTATGCTTCGGAGCACTACTCGAGTAGTGCTCCGAAGCATAGAGTTTTTTG |
| | | (SEQ ID NO: 188) |
| FGF3 | Hairpin sequence for TRCN0000038161 | CCGGCGGCAGAAGCAGAGCCCGGATCTCGAGATCCGGGCTCTGCTTCTGCCGTTTTTG |
| | | (SEQ ID NO: 189) |
| FGF3 | Hairpin sequence for TRCN0000038160 | CCGGGAGCTGGGCTATAATACGTATCTCGAGATACGTATTATAGCCCAGCTCTTTTTG |
| | | (SEQ ID NO: 190) |
| FGF3 | Hairpin sequence for V2LHS_43059 | TGCTGTTGACAGTGAGCGCCCACGAGCTGGGCTATAATACTAGTGAAGCCACAGATGTA |
| | (mir-30) | GTATTATAGCCCAGCTCGTGGATGCCTACTGCCTCGGA (SEQ ID NO: 191) |
| PPFIA1 | Hairpin sequence for TRCN0000002969 | CCGGGAGGAGATTGAAAGTCGAGTTCTCGAGAACTCGACTTTCAATCTCCTCTTTTT |
| | | (SEQ ID NO: 192) |
| PPFIA1 | Hairpin sequence for TRCN0000002967 | CCGGGTAGTTTGTTAGAAGAGGAATCTCGAGATTCCTCTTCTAACAAACTACTTTTT |
| | | (SEQ ID NO: 193) |
| PPFIA1 | Hairpin sequence for TRCN0000002968 | CCGGGCTCCAAGAAATCATAAGTAACTCGAGTTACTTATGATTTCTTGGAGCTTTTT |
| | | (SEQ ID NO: 194) |
| PPFIA1 | Hairpin sequence for TRCN0000002971 | CCGGGCACAGTTGGAGGAGAAGAATCTCGAGATTCTTCTCCTCCAACTGTGCTTTTT |
| | | (SEQ ID NO: 195) |
| PPFIA1 | Hairpin sequence for TRCN0000002970 | CCGGGCATATTAACAAGCCAGCAAACTCGAGTTTGCTGGCTTGTTAATATGCTTTTT |
| | | (SEQ ID NO: 196) |
| PPFIA1 | Hairpin sequence for V2LHS_27777 | TGCTGTTGACAGTGAGCGCCCTTGAAAGGGAAGAAGAAATTAGTGAAGCCACAGATGTA |
| | (mir-30) | ATTTCTTCTTCCCTTTCAAGGATGCCTACTGCCTCGGA (SEQ ID NO: 197) |

The linker sequences connecting the sense and anti-sense sequences are bolded.

TABLE 10
Target si RNA sequences for selected
20q13 amplicon genes

| | | |
|---|---|---|
| | siRNA | |
| Gene | no. | Target sequence |

| | | |
|---|---|---|
| BCAS1 | 1 | UAUCAGGGCAGUCCGAUGA (SEQ ID NO: 198) |
| BCAS1 | 2 | GAAGUAGAAUCAGCCUUAC (SEQ ID NO: 199) |
| BCAS1 | 3 | GAUAAGUGCUGUUGCGGAU (SEQ ID NO: 200) |
| BCAS1 | 4 | CCAAUAAAGCUCCAGCGAA (SEQ ID NO: 201) |
| CSTF1 | 1 | GGAAAUAUCAACGGGACGA (SEQ ID NO: 202) |
| CSTF1 | 2 | GGAUGGUGUUUCAAAUCGA (SEQ ID NO: 203) |
| CSTF1 | 3 | CGAAUGAUUAGUAUCGAUU (SEQ ID NO: 204) |
| CSTF1 | 4 | GUAUGCAAUUGGUCGUUCA (SEQ ID NO: 205) |
| PCK1 | 1 | CCCAAGAUCUUCCAUGUCA (SEQ ID NO: 206) |
| PCK1 | 2 | CCAUGUACGUCAUCCCAUU (SEQ ID NO: 207) |
| PCK1 | 3 | GAAGUGCUUUGCUCUCAGG (SEQ ID NO: 208) |
| PCK1 | 4 | GGUGGAAGGUUGAGUGCGU (SEQ ID NO: 209) |
| TMEPAI | 1 | GAAAGGACACCCUCUCUAG (SEQ ID NO: 210) |
| TMEPAI | 2 | GCACUUGUAAAGAUGAUUA (SEQ ID NO: 211) |
| TMEPAI | 3 | UCACUUAAGAGGCCAAUAA (SEQ ID NO: 212) |
| TMEPAI | 4 | GCGCAGAAUUCUUCACCUU (SEQ ID NO: 213) |
| RAB22A | 1 | GGACUACGCCGACUCUAUU (SEQ ID NO: 214) |
| RAB22A | 2 | GAAGAAUUCCAUCCACUGA (SEQ ID NO: 215) |
| RAB22A | 3 | GAAACAACCUCUGCGAAUU (SEQ ID NO: 216) |
| RAB22A | 4 | GCAGUUUGAUUAUCCGAUU (SEQ ID NO: 217) |
| VAPB | 1 | UGUUACAGCCUUUCGAUUA (SEQ ID NO: 218) |
| VAPB | 2 | CCACGUAGGUACUGUGUGA (SEQ ID NO: 219) |
| VAPB | 3 | GCUCUUGGCUCUGGUGGUU (SEQ ID NO: 220) |
| VAPB | 4 | GUAAUUAUUGGGAAGAUUG (SEQ ID NO: 221) |
| STX16 | 1 | GUAUGAUGUUGGCCGGAUU (SEQ ID NO: 222) |
| STX16 | 2 | AAAUAUUCCACCAGCGAUU (SEQ ID NO: 223) |
| STX16 | 3 | GCUAAUUGAGAGAGCGUUA (SEQ ID NO: 224) |
| STX16 | 4 | AGUAGGACUUCAUCGUUUA (SEQ ID NO: 225) |
| GNAS | 1 | GCAAGUGGAUCCAGUGCUU (SEQ ID NO: 226) |
| GNAS | 2 | GCAUGCACCUUCGUCAGUA (SEQ ID NO: 227) |
| GNAS | 3 | AUGAGGAUCCUGCAUGUUA (SEQ ID NO: 228) |
| GNAS | 4 | CAACCAAAGUGCAGGACAU (SEQ ID NO: 229) |

What is claimed is:

1. A method for prognosing the outcome of a patient with breast cancer, said method comprising:
providing breast cancer tissue from the patient;
determining from the provided tissue, the level of gene amplification or gene expression product for at least one gene set forth in Table 3;
identifying that the at least one gene or gene product is amplified;
whereby, when the at least one gene or gene product is amplified, this is an indication that the patient has the predicted disease free survival or probability for distant recurrence set forth in Table3.

2. The method of claim 1, wherein the gene or gene product is ACACA (SEQ ID NOs: 1, 2), ADAM9 (SEQ ID NOs: 3-8), ERBB2 (SEQ ID NOs: 9-14), FGFR1 (SEQ ID NOs: 15, 16), FNTA (SEQ ID NOs: 17, 18), IKBKB (SEQ ID NOs: 19, 20), NR1D1 (SEQ ID NOs: 21, 22), PNMT (SEQ ID NOs: 23, 24), or PROSC (SEQ ID NOs: 25, 26).

3. The method of claim 2, wherein the gene or gene product is PROSC (SEQ ID NOs: 25, 26), ADAM9 (SEQ ID NOs: 3-8), FNTA (SEQ ID NOs: 17, 18), ACACA (SEQ ID NOs: 1, 2), PNMT (SEQ ID NOs: 23, 24), or NR1D1 (SEQ ID NOs: 21, 22).

4. The method of claim 1, with a proviso that the gene or gene product is not ERBB2(SEQ ID NOs: 9-14), FGFR1 (SEQ ID NOs: 15, 16), or IKBKB (SEQ ID NOs: 19, 20).

5. The method of claim 1, wherein the gene or gene product is ADAM9 (SEQ ID NOs: 3-8).

6. The method of claim 1, wherein the gene or gene product is FGF3 (SEQ ID NOs: 65, 66), PPFIA1 (SEQ ID NOs: 69, 70), or NEU3 (SEQ ID NOs: 79, 80).

7. The method of claim 6, wherein the breast cancer is a luminal A breast cancer.

8. The method of claim 1, wherein the gene or gene product is CSTF1 (SEQ ID NOs: 117, 118), PCK1 (SEQ ID NOs: 123, 124), VAPB (SEQ ID NOs: 129, 130), GNAS (SEQ ID NOs: 135, 136), BCAS1 (SEQ ID NOs: 115, 116), TMEPA1 (SEQ ID NOs: 125, 126), or STX16 (SEQ ID NOs: 131, 132).

9. The method of claim 8, wherein the gene or gene product is GNAS (SEQ ID NOs: 135, 136), TMEPA1 (SEQ ID NOs: 125, 126), STX16 (SEQ ID NOs: 131, 132), or VAPB (SEQ ID NOs: 129, 130).

10. The method of claim 8, wherein the breast cancer is a luminal A breast cancer.

11. The method of claim 1, wherein the detecting step comprises use a of methodology selected from the group consisting of quantitative PCR, FISH, array CGH, quantitative PCR, in situ hybridization for RNA, immunohistochemistry and reverse phase protein lysate arrays for protein.

12. A method for selecting a patient for treatment with a drug that modulates the expression of a gene set forth in Table 3, said method comprising:
providing tissue biopsy from the patient;
determining from the provided tissue, the level of gene amplification or expression for a gene set forth in Table 3;
identifying that one or more of the genes is amplified or expression increased;
whereby, when the one or more genes are amplified or expression increased, this patent is a candidate for treatment with a drug that modulates the expression of the one or more gene of Table 3.

13. The method of claim 12, wherein the gene is ACACA (SEQ ID NOs: 1, 2), ADAM9 (SEQ ID NOs: 3-8), ERBB2 (SEQ ID NOs: 9-14), FGFR1 (SEQ ID NOs: 15, 16), FNTA (SEQ ID NOs: 17, 18), IKBKB (SEQ ID NOs: 19, 20), NR1D1 (SEQ ID NOs: 21, 22), PNMT (SEQ ID NOs: 23, 24), or PROSC (SEQ ID NOs: 25, 26).

14. The method of claim 13, wherein the gene is PROSC (SEQ ID NOs: 25, 26), ADAM9 (SEQ ID NOs: 3-8), FNTA (SEQ ID NOs: 17, 18), ACACA (SEQ ID NOs: 1, 2), PNMT (SEQ ID NOs: 23, 24), or NR1D1 (SEQ ID NOs: 21, 22).

15. The method of claim 12, with the proviso that the gene is not ERBB2(SEQ ID NOs: 9-14), FGFR1 (SEQ ID NOs: 15, 16), or IKBKB (SEQ ID NOs: 19, 20).

16. The method of claim 12, wherein the gene or gene product is ADAM9 (SEQ ID NOs: 3-8).

17. The method of claim 12, wherein the gene or gene product is FGF3 (SEQ ID NOs: 65, 66), PPFIA1 (SEQ ID NOs: 69, 70), or NEU3 (SEQ ID NOs: 79, 80).

18. The method of claim 12, wherein the gene or gene product is CSTF1 (SEQ ID NOs: 117, 118), PCK1 (SEQ ID NOs: 123, 124), VAPB (SEQ ID NOs: 129, 130), GNAS (SEQ ID NOs: 135, 136), BCAS1 (SEQ ID NOs: 115, 116), TMEPA1 (SEQ ID NOs: 125, 126), or STX16 (SEQ ID NOs: 131, 132).

19. The method of claim 18, wherein the gene or gene product is GNAS (SEQ ID NOs: 135, 136), TMEPA1 (SEQ ID NOs: 125, 126), STX16 (SEQ ID NOs: 131, 132), or VAPB (SEQ ID NOs: 129, 130).

20. The method of claim 12, wherein the determining step comprises use a of methodology selected from the group consisting of quantitative PCR, FISH, array CGH, quantitative PCR, in situ hybridization for RNA, immunohistochemistry and reverse phase protein lysate arrays for protein.

21. A method for treatment of a patient with breast cancer, said method comprising:
providing tissue biopsy from the patient;
determining from the provided tissue, the level of gene amplification or expression for a gene set forth in Table 3;
identifying that one or more of the genes is amplified or expression increased;
whereby, when the one or more genes are amplified or expression increased, this patent is treated with a drug that modulates the expression of the one or more gene.

22. The method of claim 21, wherein the gene is ACACA (SEQ ID NOs: 1), ADAM9 (SEQ ID NOs: 3, 5, 7), ERBB2 (SEQ ID NOs: 9, 11, 13), FGFR1 (SEQ ID NO: 15), FNTA (SEQ ID NO: 17), IKBKB (SEQ ID NO: 19), NR1D1 (SEQ ID NO: 21), PNMT (SEQ ID Ns: 23), or PROSC (SEQ ID Ns: 25).

23. The method of claim 22, wherein the gene is PROSC (SEQ ID NO: 25), ADAM9 (SEQ ID NOs: 3, 5, 7), FNTA (SEQ ID NO: 17), ACACA (SEQ ID NO: 1), PNMT (SEQ ID NO: 23), or NR1D1 (SEQ ID NO: 21).

24. The method of claim 21, with the proviso that the gene is not ERBB2(SEQ ID NOs: 9, 11, 13), FGFR1 (SEQ ID NO: 15), or IKBKB (SEQ ID NO: 19).

25. The method of claim 21, wherein the gene is ADAM9 (SEQ ID NOs: 3, 5, 7).

26. The method of claim 21, wherein the drug is an antisense sequence for a gene of Table 3, and the particular antisense sequence corresponds to the one or more amplified genes identified in the identifying step.

27. The method of claim 26, wherein the gene is FGF3 (SEQ ID NO: 65), PPFIA1 (SEQ ID NO: 69), or NEU3 (SEQ ID NO: 79).

28. The method of claim 26, wherein the gene is CSTF1 (SEQ ID NO: 117), PCK1 (SEQ ID NO: 123), VAPB (SEQ ID NO: 129), GNAS (SEQ ID NO: 135), BCAS1 (SEQ ID NO: 115), TMEPA1 (SEQ ID NO: 125), or STX16 (SEQ ID NO: 131).

29. The method of claim 28, wherein the gene is GNAS (SEQ ID NO: 135), TMEPA1 (SEq ID NO: 125), STX16 (SEQ ID NO: 131), or VAPB (SEQ ID NO: 129).

30. The method of claim 21, wherein the determining step comprises use a of methodology selected from the group consisting of quantitative PCR, FISH, array CGH, quantitative PCR, in situ hybridization for RNA, immunohistochemistry and reverse phase protein lysate arrays for protein.

31. A method for identifying a moiety that modulates a protein, said method comprising:
providing a protein selected from the group consisting of PROSC, ADAM9, FNTA, ACACA, PNMT, and NR1D1;
screening the provided protein with a candidate moiety;
determining whether the candidate moiety modulates the protein; and,
selecting a moiety that modulates the protein.

32. The method of claim 31 wherein the determining step comprises determining whether a function of the protein is modulated.

33. The method of claim 31 wherein the determining step comprises determining whether expression of the protein is modulated.

34. A method for altering the function of a PROSC, ADAM9, FNTA, ACACA, PNMT, or NR1D1 protein in a living cell, said method comprising:
providing a moiety of claim 31;
administering the moiety to a living cell that expresses PROSC, ADAM9, FNTA, ACACA, PNMT, or NR1D1 protein corresponding to the moiety;
whereby, the function of PROSC, ADAM9, FNTA, ACACA, PNMT, or NR1D1 in the cell is altered.

35. A method for prognosing the outcome of a patient with breast cancer, said method comprising:
providing breast cancer tissue from the patient;
determining from the provided tissue, the level of gene deletion for at least one gene from amplicon 8p11-12;
identifying that the at least one gene is deleted;
whereby, when the at least one gene is deleted, this is an indication that the patient has the predicted disease free survival or probability for distant recurrence set forth in Table3.

36. The method of claim 35, wherein the at least one gene from amplicon 8p11-12 is selected from the chromosome 8 genes set forth in Table 3.

37. The method of claim 35, wherein the determining step comprises use a of methodology selected from the group consisting of quantitative PCR, FISH, array CGH, quantitative PCR, in situ hybridization for RNA, immunohistochemistry and reverse phase protein lysate arrays for protein.
